(19) United States
(12) Patent Application Publication
(10) Pub. No.: US 2006/0100226 A1
(43) Pub. Date: May 11, 2006

(54) 2-Thiopyrimidinones as therapeutic agents

(76) Inventors: James A. Sikorski, Atlanta, GA (US); Jason W. Skudlarek, Suwanee, GA (US); Linda K. Hamaker, Humble, TX (US); Liming Ni, Duluth, GA (US); M. David Weingarten, Cumming, GA (US)

Correspondence Address: KING & SPALDING LLP, 191 PEACHTREE STREET, N.E., 45TH FLOOR, ATLANTA, GA, 30303-1763, US

(21) Appl. No.: 11/224,424

(22) Filed: Sep. 12, 2005

Related U.S. Application Data
(60) Provisional application No. 60/608,581, filed on Sep. 10, 2004.

(51) Int. Cl. A61K 31/513; C07D 403/02

(52) U.S. Cl. 514/269; 544/309; 544/310

(57) ABSTRACT

The present invention provides compounds of Formulas I-VII, or pharmaceutically acceptable derivatives thereof, wherein the compounds are as defined in the specification. These compounds are inhibitors of protein kinases, particularly inhibitors of MEKK protein kinases. The invention also provides pharmaceutically acceptable compositions comprising the compounds of the invention and methods of utilizing those compounds and compositions in the treatment of various protein kinase mediated disorders, such as inflammatory disorders.

### CROSS REFERENCE TO RELATED APPLICATIONS

This application claims priority to U.S. Provisional Application No. 60/608,581, filed Sep. 10, 2004.

### FIELD OF THE INVENTION

The present invention provides compounds that are protein kinase inhibitors, pharmaceutically acceptable compositions comprising these compounds, and methods of use thereof. More particularly, substituted 2-thiopyrimidin-4-ones are provided as inhibitors of MEKK protein kinases that are useful for treating a variety of diseases and conditions, such as inflammatory disorders, abnormal cellular proliferation disorders, cancer, atherosclerosis, arthritis and asthma.

### DESCRIPTION OF RELATED ART

Mitogen activated protein kinase (MAPK) signaling cascades are typically induced by extracellular signals, for example through growth factor receptors. These cascades involve a number of serine/threonine kinases that are activated by sequential phosphorylation. MAP kinases that are typically activated by extracellular signals are also referred to as extracellular signal regulated kinases (ERKs).

MAP (mitogen-activated protein) kinases (MAPKs) are components of a three kinase signaling module within the cell that also include a MAPK kinase (MAPKK) and a MAPK kinase kinase (MAPKKK, or MAP3K). MEKK (mitogen-activated protein kinase/extracellular signal-regulated kinase (ERK) kinase kinase) represents a family of related MAPK kinase kinases, including MEKK1, MEKK2, MEKK3 and MEKK4. In several cell types, disruption of MEKK activity leads to suppression of pro-inflammatory cytokine secretion. It has been proposed that inhibition of MEKK activity could lead to the development of treatments and preventive therapies for human diseases associated with MAPK signaling and pro-inflammatory cytokine secretion. Diseases associated with MAPK signaling include inflammatory diseases such as asthma, arthritis, rheumatoid arthritis, autoimmune diseases and cancer [Pelaia, G., et al., Curr. Med. Chem.—Anti-Inflammatory & Anti-Allergy Agents, 2: pp. 131-141 (2003); Dong, C., et al., Ann. Rev. Immunol., 20: pp. 55-72 (2002); Dancey, J., et al., Nature Reviews—Drug Discovery, 2: pp. 296-313; Yang, J. Y., et al., Mutation Research, 543: pp. 31-58 (2003) and Johnson, G. L., et al., Curr. Opin. Chem. Biol., 9: pp. 323-331 (2005)].

In many cell types, other signals, including stress signals, induce another kinase cascade, which leads to activation of c-Jun N-terminal kinases (JNKs). In studying the c-Jun N-terminal kinases (JNKs) with regard to their roles in cell proliferation, differentiation, and apoptosis, a full-length human MEKK2 cDNA has been cloned from Jurkat T-cells and demonstrated to be a major upstream MAPK kinase kinase for the JNK cascade in T-cells [Su, B., et al., J. Biol. Chem., 276: pp. 14784-14790 (2001)]. It was also found that in T cells, MEKK2 is a strong activator of JNK and JNK-dependent AP-1 reporter gene expression, but not of extracellular signal-regulated kinase MAPKs. These results, when taken together, illustrated that human MEKK2 is a key signaling molecule for T-cell receptor/CD3-mediated JNK MAPK activation and interleukin-2 gene expression, findings that further suggest the importance of MEKK2 in inflammatory disorders.

Further work to elucidate the in vivo function of MEKK2 [Guo, Z., et al., Molec. Cell. Biol., 22: pp. 5761-5768 (2002)] using mice carrying a targeted mutation in the MEKK locus suggested that MEKK2 may be involved in controlling the strength of T-cell receptor (TCR)/CD3 signaling.

In studying rheumatoid arthritis (RA), a chronic inflammatory disease marked by synovial lining hyperplasia and sublining infiltration with mononuclear cells and linked to the matrix metalloproteinases (MMP), it has been found that MMP production in arthritis is regulated by several signal transduction pathways including the MAPKs [Ziegler, M. E., et al., J. Cell. Physiol., 180: pp. 271-277 (1999)]. Based upon these findings, Hammaker, et al. [J. Immunol., 172: pp. 1612-1618 (2004)] demonstrated that JNK is activated in RA synovium and that this pathway regulates collagenase-1 gene expression. It was further demonstrated that regulation of JNK by MEKK2 and MAP3Ks occurs in rheumatoid arthritis, suggesting the importance of MEKK-2 as a potential therapeutic target.

Given the broad range of implications for therapeutic use of compounds capable of affecting a MAP kinase cascade, and protein kinase activation in general, there exists a need for new methods and compositions useful in the treatment of diseases and disorders which arise in connection with the activation of MAPKs, including MAP3K protein kinases and particularly MEKK2. Despite the increased attention that kinase inhibitors have received as potential human therapies over the last decade and the recent interest in identifying inhibitors of MAP3Ks, to date no inhibitors of MEKK2 activity have been reported in the literature.

Certain pyrimidinones have been reported to have antiallergic [Ban, M., et al., Bioorg. Med. Chem., 6: pp. 1057-1060 (1998)], anti-tumor agent [Wright, G. E., et al., J. Med. Chem., 27: pp. 181-189 (1984)], antipyretic [Shrivastava, S. K., et al., Bioorg. Med. Chem. Lett., 9: pp. 1885-1887 (1999)], anti-inflammatory agent [Jalander, L. F., et al., Heterocycles, 48: pp. 343-348 (1998)], and anti-parasitic [Shrivastava, S. K., et al., J. Med. Chem., 42: pp. 1667-1670 (1999)] activities.

A series of 5-cyano-6-aryl-2-thiouracils have been prepared as potentially therapeutically useful fungicidal, bactericidal, and antiviral activities [Ram, V. J., et al, Liebigs Ann. Chem., pp. 797-801 (1987)]. Following synthesis via cyclization at the N-3 or N-1 position and subsequent derivatization, all the compounds were screened for biological activity. Only one of the fifteen compounds synthesized exhibited moderate antibacterial properties against Gram-positive cocci and acid-fast bacilli, whereas all of the remaining compounds were reportedly devoid of any chemotherapeutical activity.

In searching for new antituberculosis drugs, combinatorial libraries of tetra-substituted pyrimidines, prepared via three-component condensations, were described by Kumar, et al. [Bioorg. Med. Chem. Lett., 12: pp. 667-669 (2002)]. These structurally diverse compounds had 2-substituted alkyl/aryl alkyl/cycloalkyl amines and 6-substituted aryl/substituted aryl functionalities. Biological testing of the 80 compounds generated against M. tuberculosis H37Ra cell viability showed six of the compounds to exhibit in vitro activity against this organism, thereby identifying several new anti-Mycobacterium agents which were suitable for further lead optimization studies.

Dihydroalkoxybenzyloxopyrimidines (DABOs) are a relatively new class of specific inhibitors of human immunodeficiency virus type 1 (HIV-1). Replacement of the side-chain oxygen in DABO with a sulfur atom provided thio-DABOs (S-DABOs), which exhibited an increase in anti-HIV-1 activity [Mai, A., et al., J. Med. Chem, 38: pp. 3258-3263 (1995)]. In efforts to improve the anti-HIV-1 activity of S-DABOs, modifications at position 6 of the pyrimidine ring, replacement of the benzyl functionality with smaller and/or bulkier substituents, and the introduction of a variety of substituents at the C-4 position of the pyrimidine ring have been described [Mai, A., et al., J. Med. Chem., 40: pp. 1447-1454 (1997)]. Cytotoxicity testing of the new derivatives exhibited several structural features of the compounds which are essential for anti-HIV activity.

Japanese patent application JP-1993222030A, assigned to Hisamitsu Pharmaceutical Co., Ltd., describes the preparation of a series of pyrimidin-4-ones for use as external skin preparations, specifically as tyrosinase inhibition agents and for use as skin whitening makeup. Included within the groups of compounds prepared and tested are a series of 2-thiopyrimidinones substituted at the C-6 position, as well as benzyl mercaptans.

International patent application Publication No. WO 01/07027A2 to Vertex Pharmaceuticals describes compositions and methods for inhibiting viral helicases, and in particular the hepatitis C virus NS3 helicase. Examples of compounds suitable for use in treating infections caused by poxviridae, papovaviridae, or flaviviridae viruses include both 2-amino- and 2-thio-pyrimidin-4-ones, wherein there is an electron withdrawing group at the C-5 position and the C-4 position is OH, SH, or Cl.

U.S. Patent Application Publication No. 2003/0013729 to Dr. Reddy's Laboratories, Inc., describes β-aryl-α-oxysubstituted alkylcarboxylic acids for use as hypolipidemic, antihyperglycemic compounds. These compounds are also reported to have agonist activity against PPARα and/or PPARγ, and optionally inhibit HMG CoA reductase, in addition to exhibiting agonist activity against PPARα and/or PPARγ.

Despite the increased attention that kinase inhibitors have received as potential human therapies over the last decade and the recent interest in identifying inhibitors of MAP3Ks, to date no inhibitors of MEKK2 activity have been reported in the literature.

It is therefore an object of the present invention to provide compounds, compositions, methods and uses for the treatment of cardiovascular diseases, abnormal cellular proliferation and inflammatory diseases.

It is another object of the invention to provide new compounds, compositions, method and uses for the treatment of atherosclerosis.

It is yet another object of the present invention to provide new compounds, compositions, method and uses which are inhibitors of one or more protein kinases or that inhibit activation of one or more protein kinases.

It is still another object of the present invention to provide a method for the treatment of a disease that is mediated by the expression or suppression of one or more protein kinases, and, in particular, MEKK2.

### SUMMARY OF THE INVENTION

The present invention provides novel 2-thiopyrimidin-4-ones compounds of Formulas I-VII and derivatives, analogs, tautomeric forms, stereoisomers, polymorphs, pharmaceutically acceptable salts, pharmaceutically acceptable solvates, pharmaceutically acceptable esters, and pharmaceutical compositions containing them or their mixtures useful for the inhibition of protein kinases.

In certain embodiments, the compounds of Formulas I-VII are administered to a patient in need of treatment. The 2-thiopyrimidin-4-ones of Formulas I-VII can be used to treat a patient with a variety of diseases and disorders, including but not limited to inflammatory disorders and abnormal cellular proliferation disorders. The compounds of Formulas I-VII described herein are also useful in both the primary and adjunctive medical treatment of cardiovascular disease.

In specific embodiments, the compounds can be used to treat disorders including, but not limited to, arthritis, osteoarthritis, rheumatoid arthritis, asthma, dermatitis, cystic fibrosis, post transplantation late and chronic solid organ rejection, multiple sclerosis, systemic lupus erythematosis, inflammatory bowel diseases, autoimmune diabetes, ophthalmologic disorders associated with inflammation, diabetic retinopathy, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina and small artery disease.

The compounds disclosed herein can also be used in the treatment of inflammatory skin diseases, as well as human endothelial disorders, which include, but are not limited to psoriasis, dermatitis, including eczematous dermatitis, Kaposi's sarcoma, multiple sclerosis, as well as proliferative disorders of smooth muscle cells.

The compounds can also be used, for example, in the primary treatment of disease states including atherosclerosis, post-angioplasty restenosis, coronary artery diseases and angina. The compounds of Formulas I-VII can also be administered to treat small vessel disease mediated by the activation of one or more protein kinases that is not treatable by surgery or angioplasty, or other vessel disease in which surgery is difficult or not an option. The compounds of the present invention can also be used to stabilize patients prior to revascularization therapy, and in a polymeric delivery device by being attached to a stent.

The compounds of Formulas I-VII described herein can be delivered by any appropriate administration route, for example, orally, parenterally, intravenously, intradermally, intramuscularly, subcutaneously, sublingually, transdermally, bronchially, pharyngolaryngeal, intranasally, topically such as by a cream or ointment, rectally, intraarticular, intracistemally, intrathecally, intravaginally, intraperitoneally, intraocularly, by inhalation, bucally or as an oral or nasal spray.

### DETAILED DESCRIPTION OF THE INVENTION

Compounds, pharmaceutical compositions, methods and uses for the treatment of a variety of disorders such as inflammatory disorders and abnormal cellular proliferation disorders in a subject are provided.

I. Compounds

In the embodiments shown herein, the compounds as presented are drawn as tautomers (e.g., ketone (a) and/or alcohol (b)), which are considered to be equivalent and are used interchangeably throughout the specification.

In a first embodiment, a compound of Formula I, or a pharmaceutically acceptable salt, solvate, or ester thereof is provided, 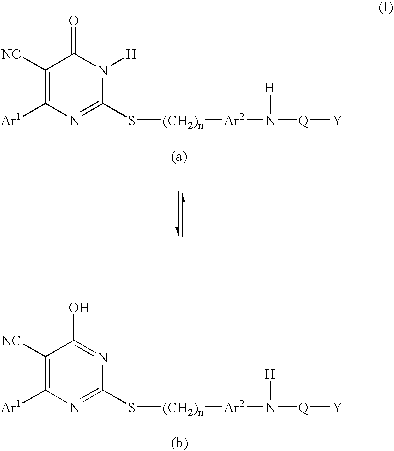
wherein:

Ar¹ is selected from an optionally substituted mono- or bicyclic aryl or an optionally substituted mono- or bicyclic heteroaryl;

Ar² is selected from an optionally substituted mono- or bicyclic aryl or an optionally substituted mono- or bicyclic heteroaryl;

n is an integer selected from 1 to 6;

Q is (CH₂)_{q}O(CH2)t or a straight chain, branched or cyclic alkyl from 1 to 10 carbon atoms, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;

q is an integer selected from 1 to 4;

t is 0 or an integer selected from 1 to 4; and

Y is selected from an optionally substituted mono- or bicyclic aryl or an optionally substituted mono- or bicyclic heteroaryl or an optionally substituted mono- or bicyclic heterocycle or an optionally substituted mono- or bicyclic alkyl.

In a second embodiment, a compound of Formula I, or a pharmaceutically acceptable salt, solvate, or ester thereof is provided,

wherein:

Ar¹ and Ar² are either the same or different and are independently selected from a mono- or bicyclic aryl or mono- or bicyclic heteroaryl which can be optionally substituted with any desired substituent, for example, by one or more independently selected from the following groups, consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;

n is an integer selected from 1 to 3;

Q is a straight chain, branched or cyclic hydrocarbon that can be saturated, unsaturated or partially unsaturated of, for example, from 1 to 8 carbon atoms, all of which can be optionally substituted with one or more groups as described previously for Ar¹ ; and

Y is selected from a mono- or bicyclic aryl, a mono- or bicyclic heteroaryl or a mono- or bicyclic heterocycle, which can be optionally substituted with any desired substituent, for example, by one or more independently selected from the following groups, consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino,. alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol.

In a third embodiment, a compound of Formula I, or a pharmaceutically acceptable salt, solvate, or ester thereof is provided,

wherein:

Ar¹, Ar², and Y are as defined in the second embodiment;

n is an integer selected from 1 to 3; and

Q is a straight chain, branched or cyclic hydrocarbon that can be saturated, unsaturated or partially unsaturated of, for example, from 1 to 4 carbon atoms, all of which can be optionally substituted with one or more groups as defined previously.

In a fourth embodiment, a compound of Formula I, or a pharmaceutically acceptable salt, solvate, or ester thereof is provided,

wherein:

Ar¹ and Ar² are as defined in the second embodiment;

n is the integer 1;

Q is a straight chain, branched or cyclic hydrocarbon that can be saturated, unsaturated or partially unsaturated of, for example, from 1 to 4 carbon atoms; and

Y is selected from a mono- or bicyclic aryl or mono- or bicyclic heteroaryl or mono- or bicyclic heterocycle that can be optionally substituted by one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a fifth embodiment, a compound of Formula II, or a pharmaceutically acceptable salt, solvate, or ester thereof is provided, 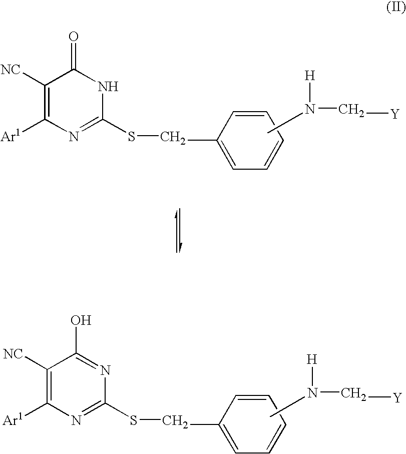
wherein:

Ar¹ is as defined in the second embodiment; and

Y is selected from a mono- or bicyclic aryl or mono- or bicyclic heteroaryl or mono- or bicyclic heterocycle that can be optionally substituted by one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a sixth embodiment, a compound of Formula II, or a pharmaceutically acceptable salt, solvate, or ester thereof is provided,

wherein:

Ar¹ is the aryl group phenyl which can be optionally substituted by one or more groups independently selected from the following: hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol; and

Y is selected from a mono- or bicyclic aryl that can be optionally substituted with one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In an seventh embodiment, a compound of Formula II, or a pharmaceutically acceptable salt, solvate, or ester thereof is provided,

wherein:

Ar¹ is as defined in the sixth embodiments; and

Y is selected from a mono- or bicyclic heteroaryl or a mono- or bicyclic heterocyclic that can be optionally substituted with one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a subembodiment of the fifth embodiment, a compound of Formula II, or a pharmaceutically acceptable salt, solvate, or ester thereof, Y is selected from aryl, substituted aryl, substituted heteroaryl, bicyclic substituted heteroaryl, heterocycle or substituted heterocyle wherein the substitutions are selected from halo, haloalkyl, alkyl, alkoxy, acyloxy, hydroxyl, heterocycle, heteroaryl, heteroaryloxy; aminoalkyl, aminoalkyl.

In another subembodiment of the fifth embodiment, a compound of Formula II, or a pharmaceutically acceptable salt, solvate, or ester thereof, Ar¹ is substituted aryl. In a specific subembodiment, Ar¹ is aryl substituted with alkoxy.

In a eighth embodiment, a compound of Formula III, or a pharmaceutically acceptable salt, solvate, or ester thereof is provided, 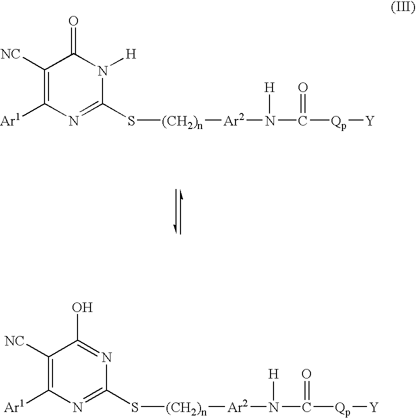
wherein:

Ar¹ is selected from an optionally substituted mono- or bicyclic aryl or an optionally substituted mono- or bicyclic heteroaryl;

Ar² is selected from an optionally substituted mono- or bicyclic aryl or an optionally substituted mono- or bicyclic heteroaryl;

n is an integer selected from 1 to 6;

Q is (CH₂)_{q}O(CH₂)ₜ or a straight chain, branched or cyclic alkyl from 1 to 10 carbon atoms, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;

q is an integer selected from 1 to 4;

t is 0 or an integer selected from 1 to 4;

p is either 0 or 1; and

Y is selected from an optionally substituted mono- or bicyclic aryl or an optionally substituted mono- or bicyclic heteroaryl or an optionally substituted mono- or bicyclic heterocycle or a mono- or bicyclic alkyl.

In a ninth embodiment, a compound of Formula III, or a pharmaceutically acceptable salt, solvate, or ester thereof is provided, wherein:

Ar¹ and Ar² are the same or different and are independently selected from a mono- or bicyclic aryl or mono- or bicyclic heteroaryl all of which can be optionally substituted with any desired substituent, for example, by one or more independently selected from the following groups, consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;

n is an integer selected from 1 to 6;

Q is a straight chain, branched or cyclic hydrocarbon of, for example, from 1 to 8 carbon atoms that can be saturated, unsaturated or partially unsaturated, all of which can be optionally substituted with one or more groups as described previously;

p is O or the integer 1; and

Y is selected from a mono- or bicyclic aryl, a mono- or bicyclic heteroaryl or a mono- or bicyclic heterocycle, all of which can be optionally substituted with any desired substituent, for example, by one or more independently selected from the following groups, consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol.

In a tenth embodiment, a compound of Formula III, or a pharmaceutically acceptable salt, solvate, or ester thereof is provided,

wherein:

Ar¹ , Ar² and Y are as defined in the ninth embodiment;

n is an integer selected from 1 to 3;

Q is a straight chain, branched or cyclic hydrocarbon of, for example, from 1 to 8 carbon atoms that can be saturated, unsaturated or partially unsaturated, all of which can be optionally substituted with one or more groups as defined above; and

p is the integer 1.

In an eleventh embodiment, a compound of Formula III, or a pharmaceutically acceptable salt, solvate, or ester thereof is provided,

wherein:

Ar¹ , Ar² and Y are as defined in the ninth embodiment;

n is an integer selected from 1 to 3;

Q is a straight chain, branched or cyclic hydrocarbon of, for example, from 1 to 4 carbon atoms that can be saturated, unsaturated or partially unsaturated, all of which can be optionally substituted with one or more groups as defined previously; and

p is the integer 1.

In a twelfth embodiment, a compound of Formula III, or a pharmaceutically acceptable salt, solvate, or ester thereof is provided,

wherein:

Ar¹ and Ar² are as defined in the ninth embodiment;

n is the integer 1;

Q is a straight chain, branched or cyclic hydrocarbon of, for example, from 1 to 4 carbon atoms that can be saturated, unsaturated or partially unsaturated;

p is the integer 1; and

Y is selected from a mono- or bicyclic aryl or mono- or bicyclic heteroaryl or mono- or bicyclic heterocycle that can be optionally substituted by one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a subembodiment of the eighth embodiment, a compound of Formula III, Y is selected from aryl, heteroaryl, substituted aryl, substituted heteroaryl, bicyclic aryl, or bicyclic heteroaryl, wherein the substitutions are selected from alkyl, alkoxy, halo, alkylhalo, aryl, heteroaryl, any of which can be optionally substituted, acyl.

In specific embodiments, Y is aryl substituted with halo, and in particular subembodiments, is aryl substituted with chloro or fluoro.

In another subembodiment of the eighth embodiment, a compound of Formula III, or a pharmaceutically acceptable salt, solvate, or ester thereof, Ar¹ is substituted aryl. In a specific subembodiment, Ar¹ is aryl substituted with alkoxy. In another specific embodiment, Ar¹ is aryl substituted with halo, alkylhalo or alkoxyhalo. In a separate embodiment, Ar¹ is aryl substituted with heteroaryl or bicyclic heteroaryl.

In a thirteenth embodiment, a compound of Formula IV, or a pharmaceutically acceptable salt, solvate, or ester thereof is provided, 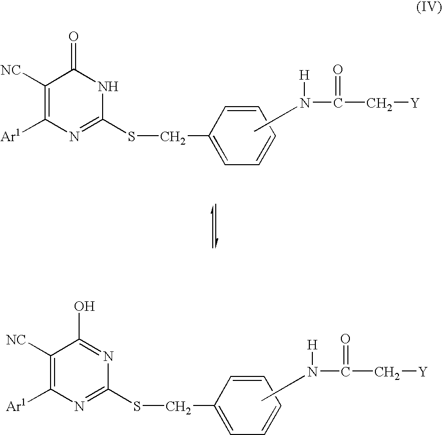
wherein:

Ar¹ is as defined in the ninth embodiment; and

Y is selected from a mono- or bicyclic aryl or mono- or bicyclic heteroaryl or mono- or bicyclic heterocycle that can be optionally substituted by one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a fourteenth embodiment, a compound of Formula IV, or a pharmaceutically acceptable salt, solvate, or ester thereof is provided,

wherein:

Ar¹ is the aryl group phenyl which can be optionally substituted by one or more groups independently selected from the following: hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol; and

Y is selected from a mono- or bicyclic aryl that can be optionally substituted with one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a fifteenth embodiment, a compound of Formula IV, or a pharmaceutically acceptable salt, solvate, or ester thereof is provided,

wherein:

Ar¹ is as defined in the fourteenth embodiment; and

Y is selected from a mono- or bicyclic heteroaryl or a mono- or bicyclic heterocyclic that can be optionally substituted with one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a sixteenth embodiment, a compound of Formula V, or a pharmaceutically acceptable salt, solvate, or ester thereof is provided, 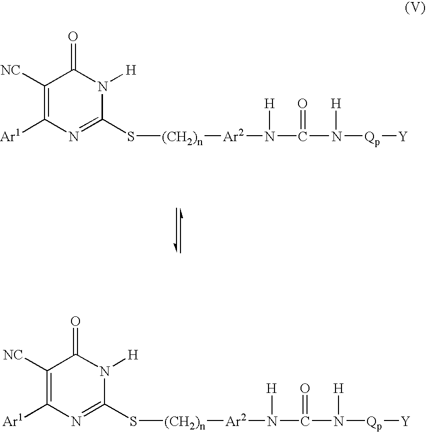
wherein:

Ar¹ is selected from an optionally substituted mono- or bicyclic aryl or an optionally substituted mono- or bicyclic heteroaryl;

Ar² is selected from an optionally substituted mono- or bicyclic aryl or an optionally substituted mono- or bicyclic heteroaryl;

n is an integer selected from 1 to 6;

Q is (CH₂)_{q}O(CH₂)ₜ or a straight chain, branched or cyclic alkyl from 1 to 10 carbon atoms, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;

q is an integer selected from 1 to 4;

t is 0 or an integer selected from 1 to 4;

p is either 0 or 1; and

Y is selected from an optionally substituted mono- or bicyclic aryl or an optionally substituted mono- or bicyclic heteroaryl or an optionally substituted mono- or bicyclic heterocycle or a mono- or bicyclic alkyl.

In a seventeenth embodiment, a compound of Formula V, or a pharmaceutically acceptable salt, solvate, or ester thereof is provided,

wherein:

Ar¹ and Ar² are either the same or different and are independently selected from a mono- or bicyclic aryl or mono- or bicyclic heteroaryl all of which can be optionally substituted with any desired substituent, for example, by one or more independently selected from the following groups, consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;

n is an integer selected from 1 to 3;

Q is a straight chain, branched or cyclic hydrocarbon that can be saturated, unsaturated or partially unsaturated of, for example, from 1 to 8 carbon atoms, all of which can be optionally substituted with one or more groups as defined previously;

p is either 0 or 1; and

Y is selected from a mono- or bicyclic aryl, a mono- or bicyclic heteroaryl or a mono- or bicyclic heterocycle, all of which can be optionally substituted with any desired substituent, for example, by one or more independently selected from the following groups, consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol.

In an eighteenth embodiment, a compound of Formula V, or a pharmaceutically acceptable salt, solvate, or ester thereof is provided,

wherein:

Ar¹ , Ar² and Y are as defined in the seventeenth embodiment;

n is an integer selected from 1 to 3;

Q is a straight chain, branched or cyclic hydrocarbon that can be saturated, unsaturated or partially unsaturated of, for example, from 1 to 4 carbon atoms, all of which can be optionally substituted with one or more groups as defined previously; and

p is either 0 or 1.

In a ninteenth embodiment, a compound of Formula V, or a pharmaceutically acceptable salt, solvate, or ester thereof is provided,

wherein:

Ar¹ , Ar² and Y are as defined in the seventeenth embodiment;

n is the integer 1;

Q is a straight chain, branched or cyclic hydrocarbon that can be saturated, unsaturated or partially unsaturated of, for example, from 1 to 4 carbon atoms, all of which can be optionally substituted with one or more groups as defined previously; and

p is either 0 or 1.

In a twentieth embodiment, a compound of Formula V, or a pharmaceutically acceptable salt, solvate, or ester thereof is provided,

wherein:

Ar¹ and Ar² are as defined in the seventeenth embodiment;

n is the integer 1;

Q is a straight chain, branched or cyclic hydrocarbon that can be saturated, unsaturated or partially unsaturated of, for example, from 1 to 4 carbon atoms, all of which can be optionally substituted with one or more groups as defined previously;

p is the integer 1; and

Y is selected from a mono- or bicyclic aryl or mono- or bicyclic heteroaryl or mono- or bicyclic heterocycle that can be optionally substituted by one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a subembodiment of the sixteenth embodiment, a compound of Formula V, Y is selected from aryl, heteroaryl, substituted aryl, substituted heteroaryl, bicyclic aryl, or bicyclic heteroaryl, wherein the substitutions are selected from alkyl, alkoxy, halo, alkylhalo, aryl, heteroaryl, any of which can be optionally substituted, acyl.

In specific embodiments, Y is aryl substituted with halo, and in particular subembodiments, is aryl substituted with chloro or fluoro.

In another subembodiment of the sixteenth embodiment, a compound of Formula V, or a pharmaceutically acceptable salt, solvate, or ester thereof, Ar¹ is substituted aryl. In a specific subembodiment, Ar¹ is aryl substituted with alkoxy.

In a twenty-first embodiment, a compound of Formula VI, or a pharmaceutically acceptable salt, solvate, or ester thereof is provided, 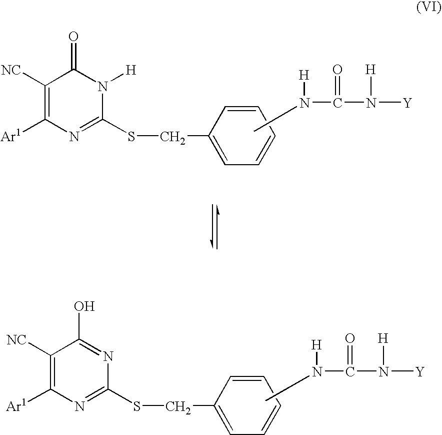
wherein:

Ar¹ is as defined in the seventeenth embodiment; and

Y is selected from a mono- or bicyclic aryl or mono- or bicyclic heteroaryl or mono- or bicyclic heterocycle that can be optionally substituted by one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a twenty-second embodiment, a compound of Formula VII, or a pharmaceutically acceptable salt, solvate, or ester thereof is provided, 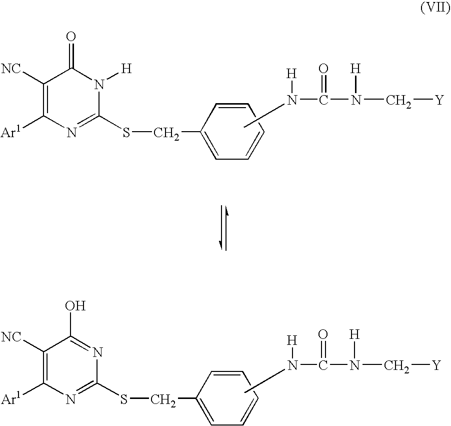
wherein:

Ar¹ is as defined in the seventeenth embodiment; and

Y is selected from a mono- or bicyclic aryl or mono- or bicyclic heteroaryl or mono- or bicyclic heterocycle that can be optionally substituted by one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a twenty-third embodiment, a compound of Formula VII, or a pharmaceutically acceptable salt, solvate, or ester thereof is provided,

wherein:

Ar¹ is the aryl group phenyl which can be optionally substituted by one or more groups independently selected from the following: hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol; and

Y is selected from a mono- or bicyclic aryl that can be optionally substituted with one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, or hydroxamic acid.

In a twenty-fourth embodiment, a compound of Formula VII, or a pharmaceutically acceptable salt, solvate, or ester thereof is provided,

wherein:

Ar¹ is as defined in the twenty-third embodiment; and

Y is selected from a mono- or bicyclic heteroaryl or a mono- or bicyclic heterocyclic that can be optionally substituted with one or more groups independently selected from the following: hydroxyl, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyol alkyl, alkylcarbonylalkyl, heteroaryl lower alkyl, heterocycle lower alkyl, heteroarylamino lower alkyl, heterocycleamino lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, amino, alkylamino, dialkylamino, alkylsulfonylamino, acylamino, arylamino, heteroarylamino, heterocycleamino, amido, imide, sulfonylimide, carboxamido, sulfonamido, acyl, aminoacyl, carboxyl, carboxylic ester, carboxylic acid, carbamate, sulfonyl, alkylsulfonyl, arylsulfonyl, aminosulfonyl, halo alkylsulfonyl, thio ester, or hydroxamic acid.

II. Definitions

The following definitions are provided in order to aid those skilled in the art in understanding the detailed description of the present invention.

The term “alkyl”, alone or in combination, means a straight, branched, or cyclic, primary, secondary, or tertiary saturated hydrocarbon, including those containing from 1 to 10 carbon atoms or from 1 to 6 carbon atoms and can be optionally substituted as described herein for “aryl”. The term alkyl includes fluorinated alkyl such as trifluoromethyl and difluoromethyl.

The term “alkenyl”, alone or in combination, means an acyclic, straight, branched, or cyclic, primary, secondary, or tertiary hydrocarbon, including those containing from 2 to 10 carbon atoms or from 2 to 6 carbon atoms, wherein the substituent contains at least one carbon-carbon double bond. These alkenyl radicals may be optionally substituted as desired for example, with groups as described above for alkyl substituents

The term “alkynyl” means an unsaturated, acyclic hydrocarbon radical, linear or branched, in so much as it contains one or more triple bonds, including such radicals containing about 2 to 10 carbon atoms or having from 2 to 6 carbon atoms. The alkynyl radicals may be optionally substituted as desired, for example with any of the groups described above for alkyl substitution. Examples of suitable alkynyl radicals include but are not limited to ethynyl, propynyl, hydroxypropynyl, butyn-1-yl, butyn-2-yl, pentyn-1-yl, pentyn-2-yl, 4-methoxypentyn-2-yl, 3-methylbutyn-1-yl, hexyn-1-yl, hexyn-2-yl, hexyn-3-yl, 3,3-dimethylbutyn-1-yl radicals and the like.

The term “acyl”, alone or in combination, means a carbonyl or thionocarbonyl group bonded to any radical to complete the valency, for example selected from, hydrido, alkyl, alkenyl, alkynyl, haloalkyl, alkoxy, alkoxyalkyl, haloalkoxy, aryl, heterocyclyl, heteroaryl, alkylsulfinylalkyl, alkylsulfonylalkyl, aralkyl, cycloalkyl, cycloalkylalkyl, cycloalkenyl, alkylthio, arylthio, amino, alkylamino, dialkylamino, aralkoxy, arylthio, and alkylthioalkyl. Examples of “acyl” are formyl, acetyl, benzoyl, trifluoroacetyl, phthaloyl, malonyl, nicotinyl, and the like.

The terms “alkoxy” and “alkoxyalkyl” includes linear or branched oxy-containing radicals each having alkyl portions of, for example, from one to about ten carbon atoms, including the methoxy, ethoxy, propoxy, and butoxy radicals. The term “alkoxyalkyl” also embraces alkyl radicals having one or more alkoxy radicals attached to the alkyl radical, that is, to form monoalkoxyalkyl and dialkoxyalkyl radicals. Other alkoxy radicals are “lower alkoxy” radicals having one to six carbon atoms. Examples of such radicals include methoxy, ethoxy, propoxy, butoxy and tert-butoxy alkyls. The “alkoxy” radicals may be further substituted with one or more halo atoms, such as fluoro, chloro or bromo, to provide “haloalkoxy” radicals. Examples of such radicals include fluoromethoxy, chloromethoxy, trifluoromethoxy, difluoromethoxy, trifluoroethoxy, fluoroethoxy, tetrafluoroethoxy, pentafluoroethoxy, and fluoropropoxy.

The term “alkylamino” includes “monoalkylamino” and “dialkylamino” radicals containing one or two alkyl radicals, respectively, attached to an amino radical. The terms “arylamino” denotes “monoarylamino” and “diarylamino” containing one or two aryl radicals, respectively, attached to an amino radical. The term “aralkylamino”, embraces aralkyl radicals attached to an amino radical, and denotes “monoaralkylamino” and “diaralkylamino” containing one or two aralkyl radicals, respectively, attached to an amino radical. The term aralkylamino further includes “monoaralkyl monoalkylamino” containing one aralkyl radical and one alkyl radical attached to an amino radical.

The term “alkoxyalkyl” is defined as an alkyl group wherein a hydrogen has been replaced by an alkoxy group. The term “(alkylthio)alkyl” is defined similarly as alkoxyalkyl, except a sulfur atom, rather than an oxygen atom, is present.

The term “alkylthio” and “arylthio” are defined as —SR, wherein R is alkyl or aryl, respectively.

The term “alkylsulfonyl” is defined as R—SO₂—, wherein R is alkyl.

The term “aryl” refers to a carbocyclic aromatic system containing one, two or three rings wherein such rings may be attached together in a pendent manner or may be fused. Examples of aryl groups include phenyl, benzyl, naphthyl, and biphenyl. The “aryl” group can be optionally substituted where desired, for example, with one or more independently selected from the following groups: of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, oxo, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy, heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol, all of which can be further substituted with one of the same substituents as set out above and can be either unprotected, or protected as necessary, as known to those skilled in the art. In addition, adjacent groups on an “aryl” ring may combine to form a 5- to 7-membered saturated or partially unsaturated carbocyclic, aryl, heteroaryl or heterocyclic ring, which in turn may be substituted as above.

The term “carbocycle”, alone or in combination, means any stable 3- to 7-membered monocyclic or bicyclic or 7- to 14-membered bicyclic or tricyclic or an up to 26-membered polycyclic carbon ring, any of which may be saturated, partially unsaturated, or aromatic. Examples of such carbocyles include, but are not limited to, cyclopropyl, cyclopentyl, cyclohexyl, phenyl, biphenyl, naphthyl, indanyl, adamantyl, or tetrahydronaphthyl (tetralin).

The term “halo” includes independently fluoro, bromo, chloro, and iodo.

The term “heterocyclic” and “heterocycle” alone or in combination includes nonaromatic cyclic groups that may be partially (e.g., contains at least one double bond) or fully saturated and wherein there is at least one heteroatom, such as oxygen, sulfur, nitrogen, or phosphorus in the ring. Similarly, the term heteroaryl or heteroaromatic, as used herein, refers to an aromatic ring that includes at least one sulfur, oxygen, nitrogen or phosphorus in the aromatic ring. Nonlimiting examples of heterocylics and heteroaromatics include pyrrolidinyl, tetrahydrofuryl, piperazinyl, piperidinyl, morpholino, thiomorpholino, tetrahydropyranyl, imidazolyl, pyrrolyl, pyrazolyl, indolyl, dioxolanyl, or 1,4-dioxanyl, aziridinyl, furyl, furanyl, chromenyl, chromenyl-4-one, pyridyl, pyrimidinyl, benzoxazolyl, 1,2,4-oxadiazolyl, 1,3,4-oxadiazolyl, 1,3,4-thiadiazole, indazolyl, 1,3,5-triazinyl, thienyl, isothiazolyl, imidazolyl, tetrazolyl, pyrazinyl, benzofuranyl, quinolinyl, isoquinolinyl, benzothienyl, isobenzofuryl, pyrazolyl, indolyl, isoindolyl, benzimidazolyl, purinyl, tetrazolyl, carbazolyl, oxazolyl, thiazolyl, benzothiazolyl, isothiazolyl, 1,2,4-thiadiazolyl, 1,2,3-thiadiazolyl, isoxazolyl, pyrrolyl, quinazolinyl, cinnolinyl, phthalazinyl, xanthinyl, hypoxanthinyl, pyrazole, imidazole, 1,2,3-triazole, 1,2,4-triazole, 1,2,3-oxadiazole, thiazine, pyridazine, or pteridinyl wherein the heteroaryl or heterocyclic group can be optionally substituted with one or more substituents, for example, one of the same substituents as set out above for aryl groups. In addition, adjacent groups on the heteroaryl or heterocyclic ring may combine to form a 5- to 7-membered carbocyclic, aryl, heteroaryl or heterocyclic ring, which in turn may be substituted as above. Functional oxygen and nitrogen groups on the heteroaryl group can be protected as necessary or as desired. Suitable protecting groups can include but are not limited to trimethylsilyl (TMS), dimethylhexylsilyl (DMHS), t-butyldimethylsilyl (TBS or TBDMS), and t-butyldiphenylsilyl (TBDPS), trityl (Trt) or substituted trityl, alkyl groups, acyl (Ac) groups such as acetyl and propionyl, methanesulfonyl, and p-toluenelsulfonyl.

The term “hydrocarbon” means a group containing only carbon and hydrogen. The term hydrocarbon as used herein includes linear, branched, or cyclic alkyl, alkenyl, alkynyl groups which may be optionally substituted, as well as aryl groups include those with a carbocyclic aromatic system containing one, two or three rings wherein such rings may be attached together in a pendent manner or may be fused.

The term “sulfonamido” includes both R—SO₂—N—, and R—N—SO₂—, wherein R is aryl, heteraryl, heterocyclic or alkyl.

The terms “protecting group” or “protected” refers to a substituent that protects various sensitive or reactive groups present, so as to prevent said groups from interfering with a reaction. Such protection may be carried out in a well-known manner as taught by Greene, et al., Protective Groups in Organic Synthesis, John Wiley and Sons, Third Edition, 1999 or the like. The protecting group may be removed after the reaction in any manner known by those skilled in the art. Non-limiting examples of protecting groups suitable for use within the present invention include but are not limited to allyl, benzyl (Bn), tertiary-butyl (t-Bu), methoxymethyl (MOM), p-methoxybenzyl (PMB), trimethylsilyl (TMS), dimethylhexylsily (TDS)l, t-butyldimethylsilyl (TBS or TBDMS), and t-butyldiphenylsilyl (TBDPS), tetrahydropyranyl (THP), trityl (Trt) or substituted trityl, alkyl groups, acyl groups such as acetyl (Ac) and propionyl, methanesulfonyl (Ms), and p-toluenesulfonyl (Ts). Such protecting groups can form, for example in the instances of protecting hydroxyl groups on a molecule: ethers such as methyl ethers, substituted methyl ethers, substituted alkyl ethers, benzyl and substituted benzyl ethers, and silyl ethers; and esters such as formate esters, acetate esters, benzoate esters, silyl esters and carbonate esters, as well as sulfonates, and borates.

III. Preparation

The compounds of this invention can be prepared by techniques from conventional organic chemistry repertoires. Schemes 1-4 below depict processes by which compounds within the scope of Formulas I-VII can be made, and are shown only for the purpose of illustration and are not to be construed as limiting the processes to make the compounds by any other methods. Exemplary compounds are not meant to limit the scope of the compounds of the present invention in any manner.

Scheme 1 schematically shows one nonlimiting method how to make a thiopyrimidinone (4) from aldehyde (1), alkyl cyanoacetate (2), and thiourea (3) using a three-component condensation in a solvent (e.g., ethanol), and in the presence of an appropriate base in a manner similar to that described by Abdou, et al. [Tetrahedron, Vol. 56: pp. 863-1836 (2000)]. The mixture in one embodiment is heated to reflux, after which the solution is allowed to cool and stand at room temperature until a precipitate forms. The solid is filtered and washed with an appropriate solvent, after which the 5-cyano-2-thio-pyrimidinone product (4) is used in the subsequent steps. Workup procedures can be modified appropriately by those of skill in the art to afford either the salt of the base used (e.g., R′ in product (4) is piperidine), or the free thiol (R′ is hydrogen).

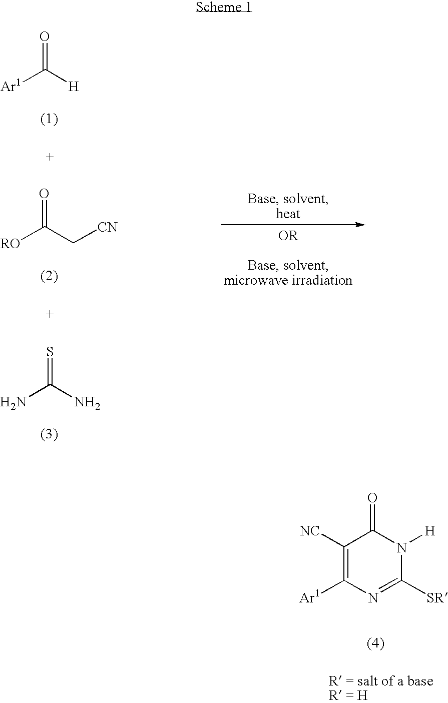

It will be appreciated by those of skill in the art that the thiopyrimidinone (4) can be prepared by other varying methods. For example, thiopyrimidinone core (4) can be prepared using a 3-component condensation using microwave radiation in a microwave synthesizer [see, Microwaves in Organic Synthesis, Loupy, A., Ed.; Wiley-VCH, Weinheim: 2002]; using solid phase synthetic methods [Bunnin, B., The Combinatorial Index, Academic Press, 1998]; or using 2-component condensations using thiourea and an appropriately substituted beta-keto ester.

The alkylation of the 2-thio functionality is shown schematically in Scheme 2 below. Two general approaches are illustrated to obtain amine (7). In a first approach, 2-thiopyrimidinone (4) is reacted with an activated alkyl aryl compound (5) having a variously substituted nitro functionality in an appropriate solvent such as N, N-dimethylformamide (DMF). The mixture is stirred at ambient temperature overnight, and is then worked up in an appropriate manner so as to generate nitro intermediate (6). The alkyl aryl compound (5) can be activated with any number of known leaving groups, including halogen (I, Br, Cl), mesylate, tosylate, and unconventional leaving groups such as the pentafluorophenyl group. Nitro intermediate (6) is then transformed to amine precursor (7) by reduction of the nitro functionality using appropriate reducing agents and conditions. It has been found that the reduction proceeds most efficiently and cleanly when it is a metal catalyzed reduction, such as by using SnCl₂, In, Ni, or the like.

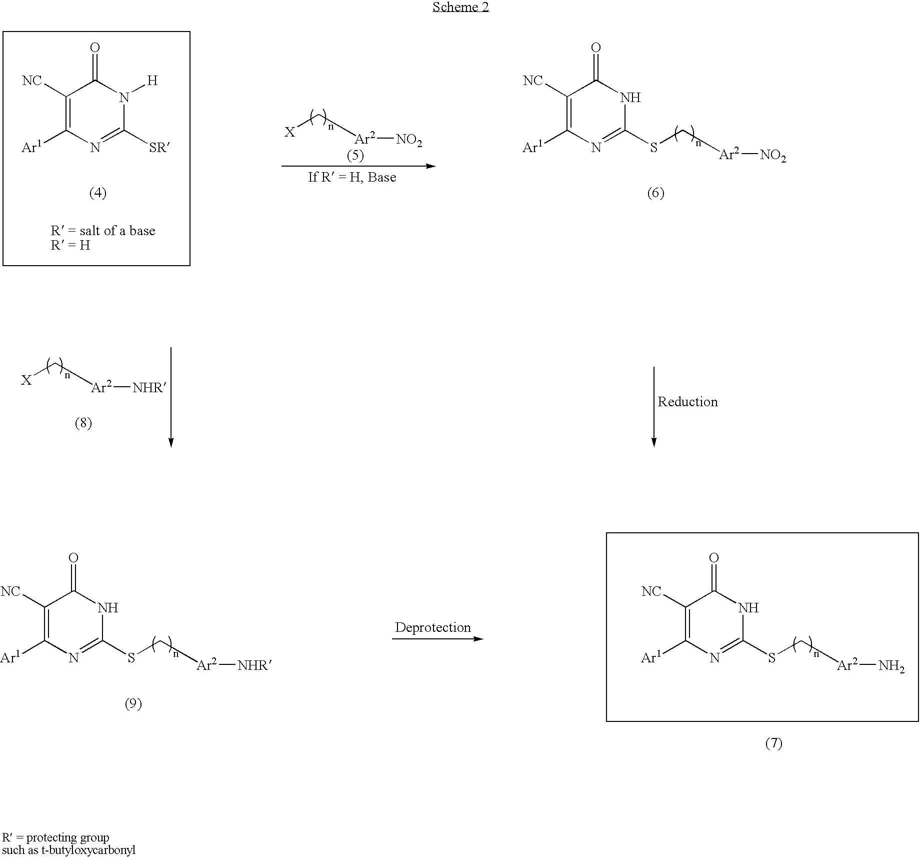

Alternatively, amine precursor (7) can be prepared by reacting 2-thiopyrimidinone (4) with an appropriately protected amino aryl alkylate (8) wherein the alkyl functionality is activated with an appropriate leaving group, such as described above, forming appropriately protected intermediate (9). The amine functionality of alkylate (8) may be protected by any of the many protecting groups known to those of skill in the art. Following isolation and purification, intermediate (9) is converted to precursor (7) by deprotection of the amine group using appropriate methodology based upon the protecting group chosen.

The amines (11), amides (15), and ureas (17) of the present invention can be prepared according to Scheme 3, as well as by other means known to those skilled in the art. As shown therein, amines (11) can be prepared by the reductive amination of amine intermediate (7) using aldehyde 12a or 12b, followed by reduction. Alternately, and equally acceptable, amines (11) can be obtained by reacting an appropriate activated alkyl functionality (13) in an appropriate solvent with a base, such as K₂CO₃. In both cases, workup and purification provide amines (11) in acceptable yields.

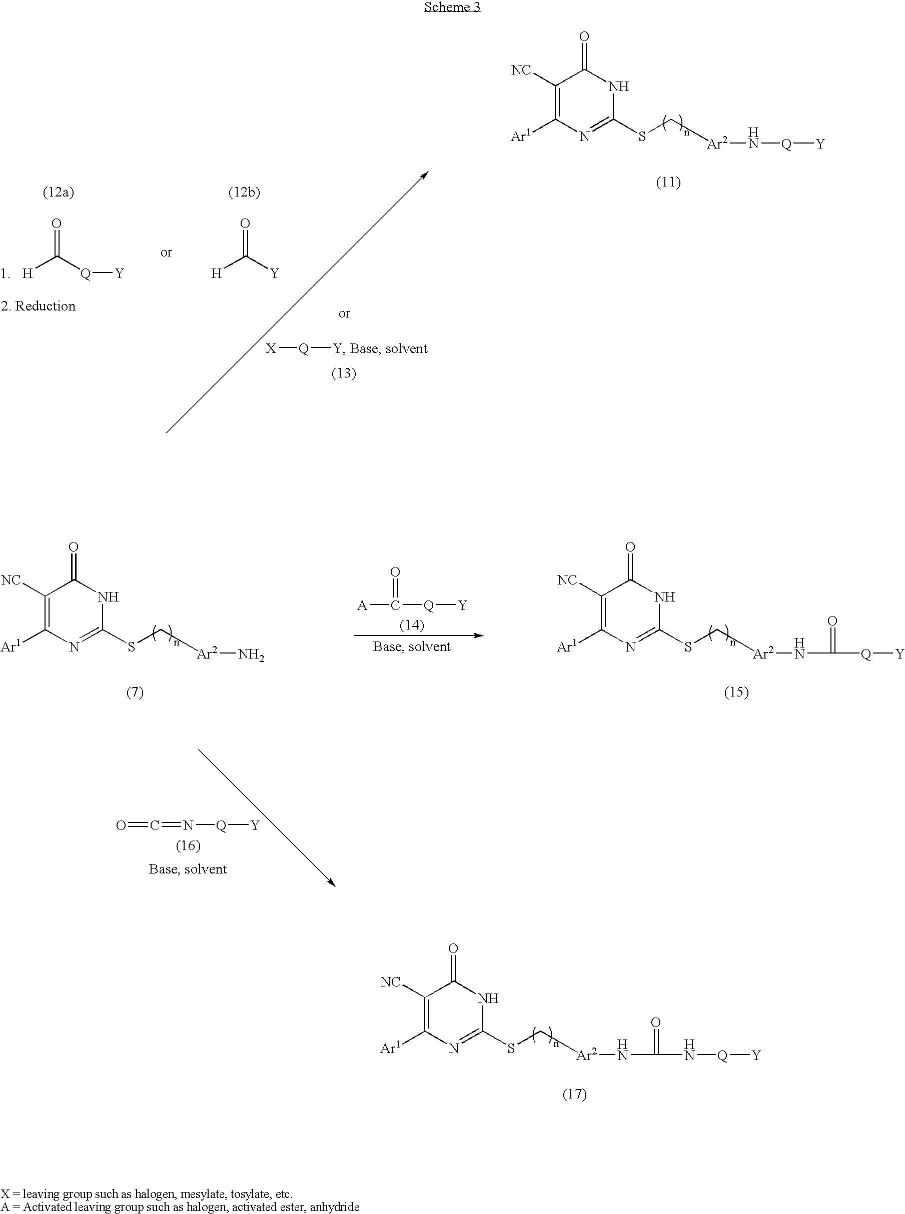

As an alternate route, amines (11) can be prepared in a convergent manner from 2-thiopyrimidinone (4) as shown in Scheme 4, below. 5-Cyano-2-thiopyrimidinone (4) is reacted with an appropriately substituted amine (10) having an activated alkyl functionality, in the presence of a base (e.g., an amine base or an alkali-metal base such as K₂CO₃ or CsCO₃) in a suitable solvent. The activated functionality is a leaving group, such as a halogen (e.g., I, Br, Cl), mesylate, tosylate, or triflate. After stirring for an appropriate period of time, the mixture is worked-up in an appropriate manner to provide amine compound (11).

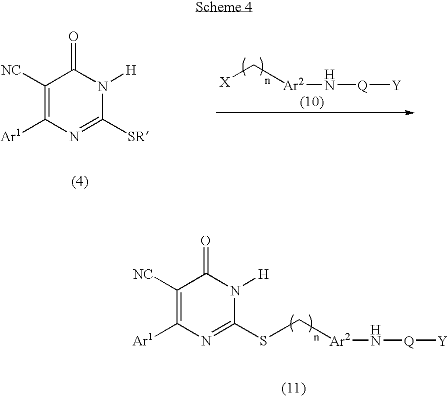

Amide (15) is prepared in a one-step reaction (Scheme 3) through the reaction of amine precursor (7) with activated carbonyl (14) and an appropriate base in a suitable solvent, such as DMF. The carbonyl may be activated in any number of ways known in the art, especially in the area of peptide chemistry, such as halides (I, Br, Cl), mesylates, tosylates, triflates, pentafluorophenyl (Pfp) esters, and the like. Amide (15) is isolated in acceptable yield following workup and purification as needed.

Urea (17) is prepared as illustrated in Scheme 3. As shown, amine precursor (7) is reacted with the appropriate isocyanate (16) in the presence of an appropriate base (e.g., pyridine) in an appropriate solvent (e.g., THF). Alternately, a catalyst such as dimethylaminopyridine (DMAP) can be added to aid in this reaction. Isolation and purification, such as by trituration, provides the target ureas in acceptable yields and high purity.

IV. Stereochemical Considerations

It is appreciated that compounds of the present invention having one or more chiral centers may exist in and be isolated in optically active and racemic forms. Some compounds may also exhibit polymorphism. It is to be understood that the present invention encompasses any racemic, optically-active, diastereomeric, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase). Examples of methods to obtain optically active materials are known in the art, and include at least the following.
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent; and
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The terms “cis” and “trans” denote a form of geometric isomerism in which two carbon atoms connected by a double bond will each have two high ranking groups on the same side of the double bond (“cis”) or on opposite sides of the double bond (“trans”). Some of the compounds described contain alkenyl groups, and are meant to include both cis and trans or “E” and “Z” geometric forms. Some of the compounds described contain one or more stereocenters and are meant to include R, S, and mixtures of R and S forms for each stereocenter present.

Some of the compounds described herein may also contain one or more ketonic or aldehydic carbonyl groups or combinations thereof alone or as part of a heterocyclic ring system. Such carbonyl groups may exist in part or principally in the “keto” form and in part or principally as one or more “enol” forms of each aldehyde and ketone group present. Compounds of the present invention having aldehydic or ketonic carbonyl groups are meant to include both “keto” and “enol” tautomeric forms.

Further, some of the compounds described herein may contain one or more imine or enamine groups or combinations thereof. Such groups may exist in part or principally in the “imine” form and in part or principally as one or more “enamine” forms of each group present. Compounds of the present invention having said imine or enamine groups are meant to include both “imine” and “enamine” tautomeric forms.

V. Therapeutic Uses

The present invention generally provides a method for treating diseases or disorders using compositions comprising the compounds of Formulas I-VII, including but not limited to inflammatory disorders, abnormal cellular proliferation disorders and cardiovascular diseases, including treatment of disorders such as atherosclerosis, diabetes, arthritis and asthma.

As a further embodiment of the present invention, a method for the treatment of an inflammatory disease in a mammal is described, comprising administering an effective amount of a compound of Formula I, Formula II, Formula III, Formula IV, Formula V, Formula VI, or Formula VII as disclosed in previous embodiments, or a pharmaceutically acceptable salt, solvate, or ester thereof, optionally with a pharmaceutically acceptable carrier, excipient or diluent, and optionally in combination and/or alternation with one or more other effective therapeutic agents for the treatment of inflammatory disorders.

In another embodiment of the present invention, the use of a compound of Formula I, Formula II, Formula III, Formula IV, Formula V, Formula VI, or Formula VII as disclosed in previous embodiments, or a pharmaceutically acceptable salt, solvate, or ester thereof, optionally with a pharmaceutically acceptable carrier or diluent, for the treatment of an inflammatory, atherosclerotic, or abnormal cellular proliferative disease or disorder in a mammal, optionally in combination and/or alternation with one or more other effective therapeutic agents, is described.

In yet a further embodiment of the present invention, the use of a compound of Formula I, Formula II, Formula III, Formula IV, Formula V, Formula VI, or Formula VII as disclosed in previous embodiments, or a pharmaceutically acceptable salt, solvate, or ester thereof, optionally in combination and/or alternation with one or more other effective therapeutic agents, and optionally with a pharmaceutically acceptable carrier or diluent, in the manufacture of a medicament for the treatment of an inflammatory, atherosclerotic, or abnormal cellular proliferative disease or disorder in a mammal is described.

The term “treatment” or “treating”, as used herein, includes an approach for obtaining beneficial or desired results including clinical results, including alleviation of symptoms, diminishment of extent of disease, stabilization (i.e., not worsening) state of disease, preventing spread of disease, preventing or reducing occurrence or recurrence of disease, delay or slowing of disease progression, and reduction of incidence of disease or symptoms. In one embodiment, the treatment is prophylactic, and, for example, the compound of the present invention is administered to prevent, or diminish the severity of, the protein kinase-related condition, for example, by administration prior to onset of disease symptoms, either before, during or after chemotherapy.

1. Inflammatory Disorders

Nonlimiting examples of inflammatory disorders that can be treated with the compounds of the present invention include immediate hypersensitivity, cytotoxic inflammation, delayed hypersensitivity inflammatory disorders, allergic or reaginic, acute inflammation, anemia, splenomegaly, hemoglobinemia, bilirubinemia, hemoblobinuria, oliguria, erythema (redness), pruritis (itch), urticaria (hives), dyspnea, rheumatic diseases, autoimmune hemolytic anemia, thrombocytopenia, immune complex inflammatory disorders glomerulonephritis, hypersensitivity pneumonitis, systemic lupus erythematosus (SLE), vaculitis, purpura hemorrhagica, anterior uveitis, arthritis, osteoarthritis, rheumatoid arthritis (RA), plasmacytic-lymphocytic synovitis, idiopathic polyarthritis, immune-mediated meningitis, Type I, Type II, Type III, and Type IV hypersensitivity reactions, atopic diseases (allergic rhinitis (hay fever/pollinosis), perennial rhinitis, allergic conjunctivitis, atopic dermatitis, angioedema, contact dermatitis, sympathetic ophthalmia, endophthalmitis phacoanaphylactica and allergic [extrinsic] asthma), urticaria and GI food reactions, systemic anaphylaxis, reactions related to exposure to water-soluble proteins in latex products (eg, rubber gloves, dental dams, condoms, tubing for respiratory equipment, catheters, and enema tips with inflatable latex cuffs), Coombs'—positive hemolytic anemias, antibody-induced thrombocytopenic purpura, leukopenia, pemphigus, pemphigoid, Goodpasture's syndrome, pernicious anemia, serum sickness due to serum, drugs, or viral hepatitis antigen; polyarteritis; cryoglobulinemia; bronchopulmonary aspergillosis; chronic membranoproliferative glomerulonephritis; and associated renal disease, allograft rejection, granulomas due to intracellular organisms, some forms of drug sensitivity, thyroiditis, and encephalomyelitis after rabies vaccination.

2. Abnormal Cellular Proliferation Disorders

The compounds of the present invention are useful to treat abnormal cellular proliferation. Nonlimiting examples of proliferative disorders are provided Table 1.

TABLE 1

| Organ System | Disease/Pathology |
|---|---|
| Dermatological | Psoriasis (all forms), acne vulgaris, acne |
| | rosacea, common warts, anogenital (venereal) |
| | warts, eczema; lupus associated skin lesions; |
| | dermatitides such as seborrheic dermatitis and |
| | solar dermatitis; keratoses such as seborrheic |
| | keratosis, senile keratosis, actinic keratosis, |
| | photo-induced keratosis, skin ageing, including |
| | photo-induced skin aging, keratosis follicularis, |
| | keloids and Prophylaxis against keloid |
| | formation; leukoplakia, lichen, planus, keratitis, |
| | contact dermatitis, eczema, urticaria, pruritus, |
| | hidradenitis, acne inversa |
| Cardiovascular | Hypertension, vasculo-occlusive diseases |
| | including Atherosclerosis, thrombosis and |
| | restenosis after angioplasty; acute coronary |
| | syndromes such as unstable angina, myocardial |
| | infarction, ischemic and non-ischemic cardio- |
| | myopathies, post-MI cardiomyopathy and |
| | myocardial fibrosis, substance-induced cardio- |
| | myopathy. |
| Endocrine | Insulin resistant states including obesity, |
| | diabetes mellitus (types 1 & 2), diabetic |
| | retinopathy, macular degeneration associated |
| | with diabetes, gestational diabetes, impaired |
| | glucose tolerance, polycystic ovarian |
| | syndrome; osteoporosis, osteopenia, accelerated |
| | aging of tissues and organs including Werner's |
| | syndrome. |
| Urogenital | Endometriosis, benign prostatic hyperplasia, |
| | leiomyoma, Polycystic kidney disease, diabetic |
| | nephropathy. |
| Pulmonary | Asthma, chronic obstructive pulmonary disease |
| | (COPD), reactive Airway disease, pulmonary |
| | fibrosis, pulmonary hypertension. |
| Connective tissue/joints | Rheumatoid arthritis, osteoarthritis, arthritis, |
| | Raynaud's phenomenon/disease, Sjogren's |
| | Syndrome, systemic sclerosis, systemic lupus |
| | erythematosus, vasculitides, ankylosing |
| | spondylitis, osteoarthritis, reactive arthritis, |
| | psoriatic arthritis, fibromyalgia. |
| Other | Fibrocystic breast disease, fibroadenoma, |
| | chronic fatigue syndrome; fibrotic disorders, |
| | hepatic cirrhosis, glomerulonephritis, malignant |
| | nephrosclerosis, thrombotic microangiopathy |
| | syndromes, transplant rejection, and |
| | glomerulopathies, Behcet's syndrome, acute |
| | respiratory distress syndrome (ARDS), |
| | ischemic heart disease, post-dialysis syndrome, |
| | acquired immune deficiency syndrome, |
| | vasculitis, lipid histiocytosis and septic shock. |

Nonlimiting examples of neoplastic diseases or malignancies are provided in Table 2.

TABLE 2

| Organ System | Malignancy/Cancer type |
|---|---|
| Skin | Basal cell carcinoma, melanoma, squamous cell |
| | carcinoma; cutaneous T cell lymphoma; Kaposi's |
| | sarcoma. |
| Hematological | Acute leukemia, chronic leukemia and myelodysplastic |
| | syndromes. |
| Urogenital | Prostatic, renal and bladder carcinomas, anogenital |
| | carcinomas including cervical, ovarian, uterine, vulvar, |
| | vaginal, and those associated with human papilloma |
| | virus infection. |
| Neurological | Gliomas including glioblastomas, astrocytoma, |
| | ependymoma, medulloblastoma, oligodendroma; |
| | meningioma, pituitary adenoma, neuroblastoma, |
| | craniopharyngioma. |
| Gastrointestinal | Colon, colorectal, gastric, esophageal, mucocutaneous |
| | carcinomas. |
| Breast | Breast cancer including estrogen receptor and |
| | progesterone Receptor positive or negative subtypes, |
| | soft tissue tumors. |
| Metastasis | Metastases resulting from the neoplasms. |
| Skeletal | Osteogenic sarcoma, malignant fibrou histeocytoma, |
| | chondrosarcoma, rhabdomyosarcoma, leiomyosarcoma, |
| | myeloma. |
| Diffuse Tumors | Lymphoma (non-Hodgkin's or Hodgkin's), sickle |
| | cell anemia. |
| Other | Angiomata, angiogenesis associated with the neoplasms. |

Nonlimiting examples of neoplastic diseases or malignancies (e.g., tumors) treatable with the compounds of the present invention include but are not limited to the following:
(i) benign tumors, including, but not limited to papilloma, adenoma, firoma, chondroma, osteoma, lipoma, hemangioma, lymphangioma, leiomyoma, rhabdomyoma, neuroma, ganglioneuroma, nevus, pheochromocytoma, neurilemona, fibroadenoma, teratoma, hydatidiform mole, granuosa-theca, Brenner tumor, arrhenoblastoma, hilar cell tumor, sex cord mesenchyme, interstitial cell tumor and thyoma;
(ii) malignant tumors (cancer), including but not limited to carcinoma, including renal cell carcinoma, prostatic adenocarcinoma, bladder carcinoma,and adenocarcinoma, fibrosarcoma, chondrosarcoma, osteosarcoma, liposarcoma, hemangiosarcoma, lymphangiosarcoma, leiomyosarcoma, rhabdomyosarcoma, myelocytic leukemia, erythroleukemia, multiple myeloma, glioma, meningeal sarcoma, thyoma, cystosarcoma phyllodes, nephroblastoma, teratoma choriocarcinoma, cutaneous T-cell lymphoma (CTCL), cutaneous tumors primary to the skin (for example, basal cell carcinoma, squamous cell carcinoma, melanoma, and Bowen's disease), breast and other tumors infiltrating the skin, Kaposi's sarcoma, and premalignant and malignant diseases of mucosal tissues, including oral, bladder, and rectal diseases, central nervous system tumors (glioblastomas), meningiomas, and astrocytomas; and
(iii) hyperproliferative and preneoplastic lesions, including mycosis fungoides, dermatomyositis, viruses (for example, warts, herpes simplex, and condyloma acuminata), molluscum contagiosum, remalignant and malignant diseases of the female genital tract (cervix, vagina, and vulva).
   3. Atherosclerosis and Angiogenic-Related Diseases

In accordance with such antiangiogenic behavior, it is expected that compounds of the present invention can be used in the treatment of angiogenic-related diseases including but not limited to: diseases associated with M-protein; cancers and tumors, such as those described previously and listed above; liver diseases; von-Hippel-Lindau disease; VEGF-related diseases and disorders; and numerous vascular (blood-vessel) diseases, which include but are not limited to abetalipoproteinemia; aneurysms; angina (angina pectoris), antiphospholipid syndrome; aortic stenosis; aortitis; arrhythmias; arteriosclerosis; arteritis; Asymmetric Septal Hypertrophy (ASH); atherosclerosis; athletic heart syndrome; atrial fibrillation; bacterial endocarditis; Barlow's Syndrome (Mitral Valve Prolapse); bradycardia; Buerger's Disease (Thromboangitis Obliterans); cardiac arrest; cardiomegaly; cardiomyopathy; carditis; carotid artery disease; high blood cholesterol; coarctation of the aorta; congenital heart diseases (congenital heart defects); congestive heart failure; coronary artery disease; coronary heart disease; Eisenmenger's Syndrome; embolism; endocarditis; erythromelalgia; fibrillation; myocardial infarction; congential heart disease; heart murmurs; hemangiomas; hypercholesterolemia; hyperlipidemia; hyperipoproteinemia; hypertriglyceridemia; hypertension; hypercholesterolemia Familial; renovascular hypertension; steroid hypertension; hypobetalipoproteinema; hypolipoproteinemia; hypotension (low blood pressure); idiopathic infantile arterial calcification; Kawasaki Disease (Mucocutaneous Lymph Node Syndrome, Mucocutaneous Lymph Node Disease, Infantile Polyarteritis); lipid transport disorders; metabolic syndrome; microvascular angina; myocarditis; paroxysmal atrial tachycardia (PAT); periarteritis nodosa (Polyarteritis, Polyarteritis Nodosa); Pericardial Tamponade; pericarditis; peripheral vascular disease; pheochromocytoma; phlebitis; pulmonary valve stenosis; Raynaud's disease; renal artery stenosis; rheumatic heart disease; septal defects; silent ischemia; sudden cardiac death; syndrome X; tachycardia; Takayasu's arteritis; Tetralogy of Fallot; thrombembolism; thrombosis; transposition of the Great Vessels; tricuspid atresia; truncus arteriosus; varicose ulcers; varicose veins; vasculitis; ventricular septal defect; Wolff-Parkinson-White Syndrome; and Xanthomatosis (Familial hypercholesterolemia, Type II hyperlipoproteinemia; Hypercholesterolemic Xanthomatosis).

4. Combination Therapy

The compounds of this invention may be used in combination with other drugs and therapies used in the treatment of disease states which would benefit from the inhibition of cytokines, in particular TNF-α and protein kinases. For example, the compounds of the Formula I-VII could be used in combination with drugs and therapies used in the treatment of inflammatory diseases, cardiovascular diseases, rheumatoid arthritis, asthma, cancer, ischaemic heart disease, psoriasis and the other disease states mentioned earlier in this specification.

For example, the compounds of Formula I-VII are of value in the treatment of certain inflammatory and non-inflammatory diseases which are currently treated with a cyclooxygenase-inhibitory non-steroidal anti-inflammatory drug (NSAID) such as indomethacin, ketorolac, acetylsalicylic acid, ibuprofen, sulindac, tolmetin and piroxicam. Co-administration of a compound of the Formula I-VII with an NSAID can result in a reduction of the quantity of the latter agent needed to produce a therapeutic effect. Thereby, the likelihood of adverse side-effects from the NSAID such as gastrointestinal effects are reduced. Thus, according to a further feature of the invention there is provided a pharmaceutical composition which comprises a compound of Formula I-VII or a pharmaceutically acceptable salt, solvate, or in vivo cleavable ester thereof, in conjunction or admixture with a cyclooxygenase inhibitory non-steroidal anti-inflammatory agent, and a pharmaceutically acceptable diluent or carrier.

The compounds of Formula I-VII may also be used in the treatment of conditions such as rheumatoid arthritis in combination with antiarthritic agents such as gold, methotrexate, steroids and penicillinamine, and in conditions such as osteoarthritis in combination with steroids. Similarly, the compounds of Formula I-VII may be used in the treatment of asthma in combination with antiasthmatic agents such as bronchodilators and leukotriene antagonists.

With regard to abnormal cellular proliferation diseases, the compounds of Formula I-VII may be used in the treatment of abnormal cellular proliferation diseases and disorders in combination with a number of known agents suitable for use in the treatment of such diseases. Such agents include but are not limited to Aceglatone; Aclarubicin; Altretamine; Aminoglutethimide; 5-Aminogleavulinic Acid; Amsacrine; Anastrozole; Ancitabine Hydrochloride; 17-1A Antibody; Antilymphocyte Immunoglobulins; Antineoplaston A10; Asparaginase; Pegaspargase; Azacitidine; Azathioprine; Batimastat; Benzoporphyrin Derivative; Bicalutamide; Bisantrene Hydrochloride; Bleomycin Sulphate; Brequinar Sodium; Broxuridine; Busulphan; Campath-IH; Caracemide; Carbetimer; Carboplatin; Carboquone; Carmofuir; Carmustine; Chlorambucil; Chlorozotocin; Chromomycin; Cisplatin; Cladribine; Corynebacterium parvum; Cyclophosphamide; Cyclosporin; Cytarabine; Dacarbazine; Dactinomycin; Daunorubicin Hydrochloride; Decitabine; Diaziquone; Dichlorodiethylsulphide; Didemnin B.; Docetaxel; Doxifluridine; Doxorubicin Hychloride; Droloxifene; Echinomycin; Edatrexate; Elliptinium; Elmustine; Enloplatin; Enocitabine; Epirubicin Hydrochloride; Estramustine Sodium Phosphate; Etanidazole; Ethoglucid; Etoposide; Fadrozole Hydrochloride; Fazarabine; Fenretinide; Floxuridine; Fludarabine Phosphate; Fluorouracil; Flutamide; Formestane; Fotemustine; Gallium Nitrate; Gencitabine; Gusperimus; Homoharringtonine; Hydroxyurea; Idarubicin Hydrochloride; Ifosfamide; Ilmofosine; Improsulfan Tosylate; Inolimomab; Interleukin-2; Irinotecan; JM-216; Letrozole; Lithium Gamolenate; Lobaplatin; Lomustine; Lonidamine; Mafosfamide; Melphalan; Menogaril; Mercaptopurine; Methotrexate; Methotrexate Sodium; Miboplatin; Miltefosine; Misonidazole; Mitobronitol; Mitoguazone Dihydrochloride; Mitolactol; Mitomycin; Mitotane; Mitozanetrone Hydrochloride; Mizoribine; Mopidamol; Multialchilpeptide; Muromonab-CD3; Mustine Hydrochloride; Mycophenolic Acid; Mycophenolate Mofetil; Nedaplatin; Nilutamide; Nimustine Hydrochloride; Oxaliplatin; Paclitaxel; PCNU; Penostatin; Peplomycin Sulphate; Pipobroman; Pirarubicin; Piritrexim Isethionate; Piroxantrone Hydrochloride; Plicamycin; porfimer Sodium; Prednimustine; Procarbazine Hydrochloride; Raltitrexed; Ranimustine; Razoxane; Rogletimide; Roquinimex; Sebriplatin; Semustine; Sirolimus; Sizofiran; Sobuzoxane; Sodium Bromebrate; Sparfosic Acid; Sparfosate Sodium; Sreptozocin; Sulofenur; Tacrolimus; Tamoxifen; Tegafur; Teloxantrone Hydrochloride; Temozolomide; Teniposide; Testolactone; Tetrasodium Meso-tetraphenylporphinesulphonate; Thioguanine; Thioinosine; Thiotepa; Topotecan; Toremifene; Treosulfan; Trimetrexate; Trofosfamide; Tumor Necrosis Factor; Ubenimex; Uramustine; Vinblastine Sulphate; Vincristine Sulphate; Vindesine Sulphate; Vinorelbine Tartrate; Vorozole; Zinostatin; Zolimomab Aritox; and Zorubicin Hydrochloride, as well as combinations of one or more of any of these agents.

VI. Biological Activity

In practicing various aspects of the present invention, compounds in accordance with the invention can be tested for a biological activity of interest using any assay protocol that is predictive of activity in vivo. For example, a variety of convenient assay protocols are available that are useful in measuring MEKK-2 inhibitory activity in vivo.

In one approach, MEKK2 inhibitory activity of compounds of the invention can be assessed using the time-resolved Fluorescence Resonance Energy Transfer (TR-FRET) assay detailed in Example 5. In this assay, the excitation energy of one fluorescent molecule (the donor) is transferred by a resonance mechanism to a nearby second fluorescent molecule (the acceptor), which then releases its fluorescent energy through fluorescent emission. This fluorescent energy is measured using a time-resolved fluorescence measurement protocol (LANCE high count 615/665); excitation occurred with 1,000 flashes at 325 nm, measurement was delayed by 100 μs, and data were acquired for 50 μs at 615 and 665 nm. Accordingly, measurement of this fluorescent energy provides useful binding date for possessing potential specific protein kinase affinity.

Another useful method for assessing protein kinase inhibition, and specifically MEKK2 inhibition, of compounds of the invention involves the MEKK2 phospho-antibody enzyme-linked immunosorbent assay (PhosphoELISA). This screening panel, which involves relatively few steps, is a useful indicator of potential inhibitors of MEKK2. Reactions to murine MEKK2 are observed as optical density measurements at an absorbance of 450 nm. Inhibitory activities can be expressed in terms of IC₅₀, where IC₅₀ is the molar concentration of compound required to inhibit protein kinase binding by 50%.

Experiments conducted in support of the present invention demonstrate that certain compounds of the present invention exhibit activity in several MEKK2 inhibitory assays, as summarized in the Examples.

VII. Pharmaceutical Compositions and Administration

In a further embodiment of the present invention, a pharmaceutical composition for the treatment and/or prophylaxis of inflammatory disorders, abnormal cellular proliferation disorders, atherosclerosis, diabetes, arthritis and asthma is described, the composition comprising a compound of Formula I, Formula II, Formula III, Formula IV, Formula V, Formula VI, or Formula VII as disclosed herein in any of the previous embodiments, or a pharmaceutically acceptable salt, solvate, or ester thereof, optionally with a pharmaceutically acceptable carrier or diluent, and optionally with one or more other effective therapeutic agents.

In another embodiment of the present invention, a pharmaceutical composition for the treatment and/or prophylaxis of inflammatory disorders, abnormal cellular proliferation disorders, atherosclerosis, diabetes, arthritis and asthma is described, the composition comprising a compound of Formula I, Formula II, Formula III, Formula IV, Formula V, Formula VI, or Formula VII as disclosed herein in any of the previous embodiments, or a pharmaceutically acceptable salt, solvate, or ester thereof, optionally with a pharmaceutically acceptable carrier or diluent, and optionally with one or more other effective therapeutic agents for the treatment of inflammatory disorders.

Patients, including mammals and particularly humans, suffering from any of the disorders described herein, including abnormal cellular proliferation disorders, atherosclerosis, diabetes, asthma, and inflammatory disorders, can be treated by administering to the host an effective amount of a compound of Formula I, Formula II, Formula III, Formula IV, Formula V, Formula VI, or Formula VII, as described herein, or a pharmaceutically acceptable salt, solvate, or ester, thereof, optionally in combination with a pharmaceutically acceptable carrier or diluent.

The compounds of the invention can be administered by any appropriate administration route, for example, orally, parenterally, intravenously, intradermally, intramuscularly, subcutaneously, sublingually, transdermally, bronchially, pharyngolaryngeal, intranasally, topically such as by a cream or ointment, rectally, intraarticular, intracistemally, intrathecally, intravaginally, intraperitoneally, intraocularly, by inhalation, bucally or as an oral or nasal spray. The route of administration may vary, depending upon the condition and the severity of the disease or disorder. The precise amount of compound administered to a host or patient will be the responsibility of the attendant physician. However, the dose employed will depend on a number of factors, including the age and sex of the patient, the precise disorder being treated, and its severity.

The invention also contemplates the use of these compounds in in vitro cellular assays to study the mechanism of protein kinases and metabolism.

The compounds of the present invention can be used in the form of pharmaceutically acceptable salts derived from inorganic or organic acids. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zünch, Switzerland: 2002). The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, flimarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids can also be made.

The formulations may conveniently be presented in unit dosage form and may be prepared by any of the methods well known in the art of pharmacy. All methods include the step of bringing into association a compound of the invention or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

The compound or a pharmaceutically acceptable ester, salt, solvate or prodrug can be mixed with other active materials that do not impair the desired action, or with materials that supplement the desired action, including other drugs against diabetic vascular disease or ocular inflammatory disease. Solutions or suspensions used for parenteral, intradermal, subcutaneous, or topical application can include, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, carriers can be physiological saline or phosphate buffered saline (PBS).

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

Besides inert diluents, the formulation compositions can also include adjuvants such as wetting agents, emulsifying and suspending agents, sweetening, flavoring, and perfuming agents. The active compounds can also be in micro-or nano-encapsulated form, if appropriate, with one or more excipients.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Formulations for parenteral (including subcutaneous, intradermal, intramuscular, intravenous and intraarticular) administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Yet another aspect of the present invention involves formulating the compounds of Formulas I-VII using polymers such as biopolymers or biocompatible (synthetic or naturally occurring) polymers. Biocompatible polymers can be categorized as biodegradable and non-biodegradable. Biodegradable polymers degrade in vivo as a function of chemical composition, method of manufacture, and implant structure. Illustrative examples of synthetic polymers include polyanhydrides, polyhydroxyacids such as polylactic acid, polyglycolic acids and copolymers thereof, polyesters polyamides polyorthoesters and some polyphosphazenes. Illustrative examples of naturally occurring polymers suitable for use with the present invention include proteins and polysaccharides such as collagen, hyaluronic acid, albumin, and gelatin.

The amount of active ingredient that may be combined with the carrier materials to produce a single dosage form will vary depending upon the subject treated and the particular mode of administration. In general, however, the amount of active ingredient administered to a subject will be an amount sufficient to be considered a therapeutically effective dose. Tablets or other forms of dosage presentation provided in discrete units may conveniently contain an amount of one or more of the compounds of the invention which are effective at such dosage rages, or ranges in between these ranges.

A therapeutically effective dose, as used herein, refers to that amount of the compound that results in achieving the desired effect. The dosage can vary within the effective range depending upon the dosage form employed, and the route of administration utilized.

The compounds and formulations of the present invention can be administered in any of the known dosage forms standard in the art.

Solid dosage forms for oral administration include capsules, caplets, tablets, pills, powders, lozenges, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Semi-liquid dosage forms include those dosage forms that are too soft in structure to qualify for solids, but to thick to be counted as liquids. These include creams, pastes, ointments, gels, lotions, and other semisolid emulsions containing the active compound of the present invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Dosage forms for topical or transdermal administration of a compound of this invention include ointments, pastes, creams, lotions, gels, powders, solutions, sprays, inhalants or patches, optionally mixed with degradable or nondegradable polymers. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

Formulations containing compounds of the invention may be administered through the skin by an appliance such as a transdermal patch. Patches can be made of a matrix such as polyacrylamide, polysiloxanes, or both and a semi-permeable membrane made from a suitable polymer to control the rate at which the material is delivered to the skin. Other suitable transdermal patch formulations and configurations are described in U.S. Pat. Nos. 5,296,222 and 5,271,940, as well as in Satas, D., et al, “Handbook of Pressure Sensitive Adhesive Technology, 2^{nd} Ed.”, Van Nostrand Reinhold, 1989: Chapter 25, pp. 627-642.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. Such excipients are described, for example, in “Handbook of Pharmaceutical Excipients, 3^{rd} Ed.”, A. H. Kibbe, Ed. (American Pharmaceutical Association and Pharmaceutical Press, Washington, D.C., 2000), the entire contents of which are included herein by reference.

The active compounds of the present invention can be prepared with carriers that will protect the compound against rapid elimination from the body or rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Methods for preparation of such formulations will be apparent to those skilled in the art. Other controlled-release formulations include but are not limited to the use of nanospheres, nanoparticles such as gelatin nanoparticles, polyacrylics, polymers such as poly(acrylamide-co-styrene) and polyvinyl alcohol, controlled-release glass, cellulose and cellulose derivatives, and biodegradable controlled release formulations such as hydrophilic-hydrophobic hydrogels.

### EXAMPLES

The following examples are included to demonstrate embodiments of the invention. The examples are understood to be illustrative only and are not intended to limit the scope of the present invention in any way. It should be appreciated by those of skill in the art that the techniques disclosed in the examples which follow represent techniques discovered by the inventors to function well in the practice of the invention, and thus can be considered to constitute examples of modes for its practice. However, those of skill in the art should, in light of the present disclosure, appreciate that many changes can be made in the specific embodiments which are disclosed and still obtain a like or similar result without departing from the scope of the invention.

General Techniques. Unless noted otherwise, materials were obtained from commercially available sources and used without further purification. Benzene, dichloromethane (CH₂Cl₂), acetonitrile (CH₃CN), triethylamine (Et₃N), tetrahydrofuran (THF), dimethylformamide (DMF) and pyridine are anhydrous.

Melting points are uncorrected. Nuclear magnetic resonance (NMR) spectra were recorded on either a Bruker Avance 300 at 300 MHz or General Electric QE-300 magnetic resonance spectrometer. ¹H chemical shifts are given in parts per million (ppm, δ) downfield from tetramethylsilane (TMS) using the residual solvent signal (CHCl₃=δ 7.27, benzene=δ 7.15, acetone=δ 2.04) as internal standard. Proton (¹H) NMR information is tabulated in the following format: multiplicity (s, singlet; d, doublet; t, triplet; q, quartet; sept, septet, m, multiplet), number of protons, coupling constant(s) (J) in hertz and, in cases where mixtures are present, assignment as the major or minor isomer, if possible. The prefix “app” (approximate) is occasionally applied in cases where the true signal multiplicity was unresolved and “br” indicates the signal in question was broadened. Proton decoupled ¹³C NMR spectra are reported in ppm (δ) relative to residual CHCl₃ (δ 77.25) unless noted otherwise.

Mass spectra were obtained on either a VG 70S (for El) or Micromass Q-TOF (for ES) or on a PE-SCIEX API 150EX instrument.

Elemental analyses (C, H, N, S) were performed by Atlantic Microlabs, Norcross, Georgia. Silica gel 60 (E. Merck, 230-400 mesh) was used for flash column chromatography (according to the protocol of Still, W. C., et al. [μJ. Org. Chem, 43: pp. 2923-2925 (1978)]. Column fractions were followed using Thin Layer Chromatography visualized under 254 nm UV light.

For binding and cell-inhibition studies, procedures were as described in the testing section above, and in Examples 5 and 6 below.

### Example 1

### Preparation of Examples 1a through 1dd

| | | Mass |
|---|---|---|
| | | Spec |
| Example | Structure | Data |
| 1a | 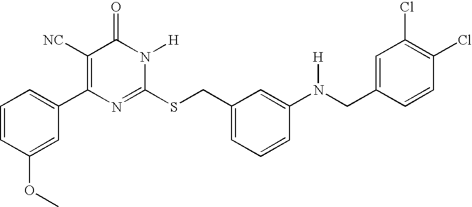 | HRMS (EI⁺) m/z: calc. 523.0762, found 523.0781 |
| 1b | 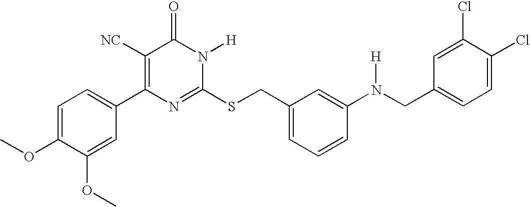 | HRMS (EI⁺) m/z: calc. 523.0868, found 523.0852 |
| 1c | 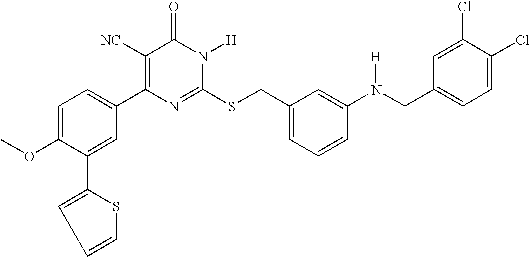 | HRMS (EI⁺) m/z: calc. 604.0561, found 604.0576. |
| 1d | 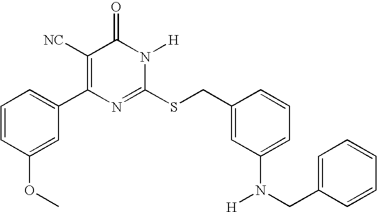 | APCI MS m/z 455.2 [M + H]⁺ |
| 1e | 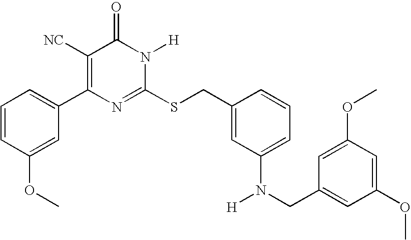 | APCI MS m/z 515.4 [M + H]⁺ |
| 1f | 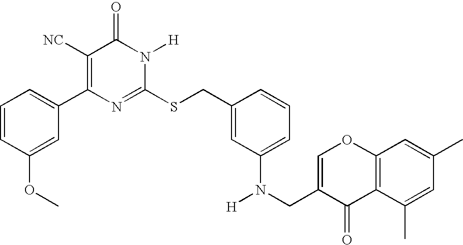 | APCI MS m/z 551.2 [M + H]⁺ |
| 1g | 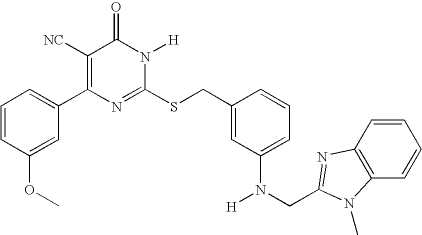 | APCI MS m/z 509.2 [M + H]⁺ |
| 1h | 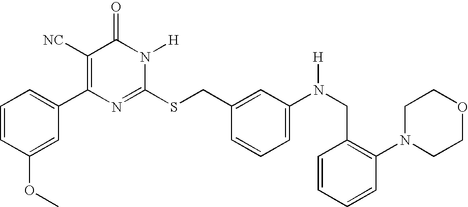 | APCI MS m/z 540.4 [M + H]⁺ |
| 1i | 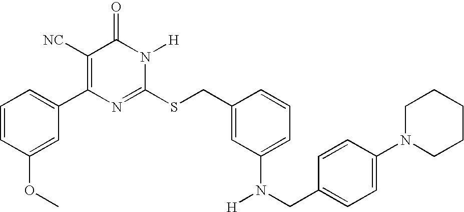 | APCI MS m/z 538.4 [M + H]⁺ |
| 1j | 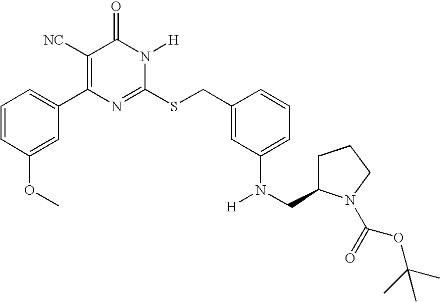 | APCI MS m/z 548.4 [M + H]⁺ |
| 1k | 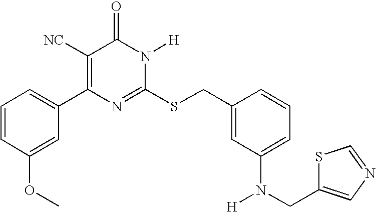 | APCI MS m/z 462.2 [M + H]⁺ |
| 1l | 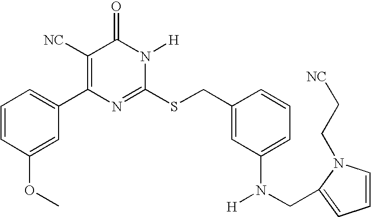 | APCI MS m/z 497.2 [M + H]⁺ |
| 1m | 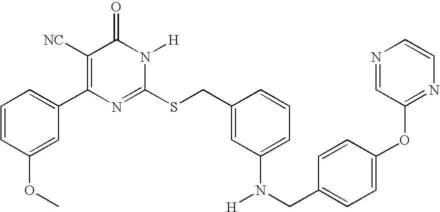 | APCI MS m/z 549.2 [M + H]⁺ |
| 1n | 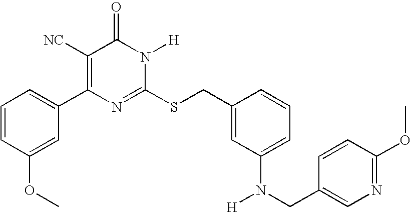 | APCI MS m/z 486.2 [M + H]⁺ |
| 1o | 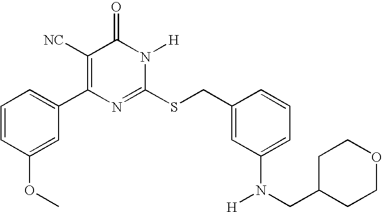 | APCI MS m/z 463.4 [M + H]⁺ |
| 1p | 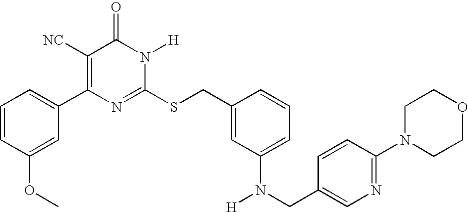 | APCI MS m/z 541.4 [M + H]⁺ |
| 1q | 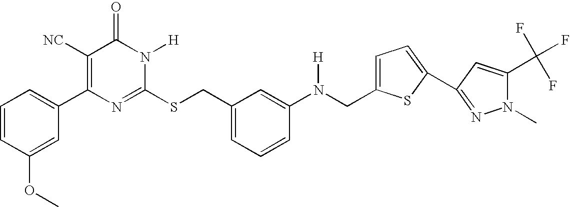 | APCI MS m/z 609.2 [M + H]⁺ |
| 1r | 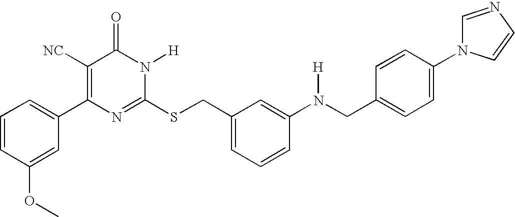 | APCI MS m/z 521.0 [M + H]⁺ |
| 1s | 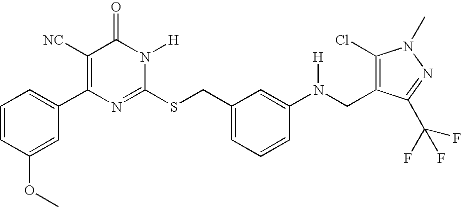 | APCI MS m/z 561.0 [M + H]⁺ |
| 1t | 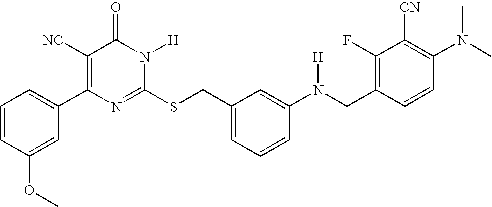 | APCI MS m/z 541.0 [M + H]⁺ |
| 1u | 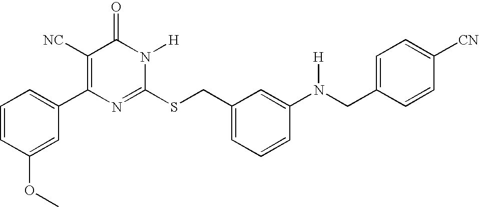 | APCI MS m/z 480.0 [M + H]⁺ |
| 1v | 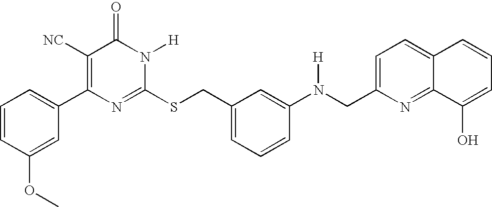 | APCI MS m/z 522.2 [M + H]⁺ |
| 1w | 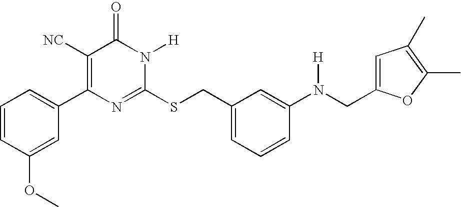 | APCI MS m/z 471.2 [M + H]⁺ |
| 1x | 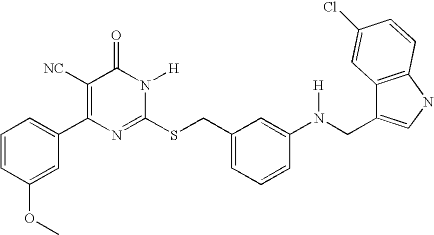 | APCI MS m/z 528.2 [M + H]⁺ |
| 1y | 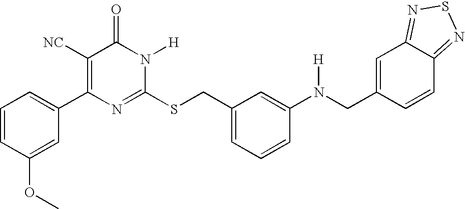 | APCI MS m/z 513.0 [M + H]⁺ |
| 1z | 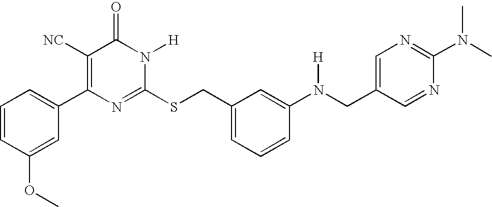 | APCI MS m/z 500.2 [M + H]⁺ |
| 1aa | 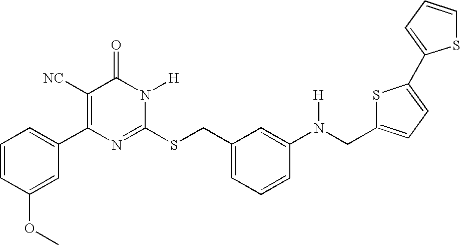 | APCI MS m/z 543.2 [M + H]⁺ |
| 1bb | 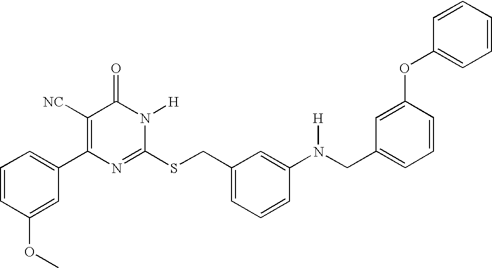 | APCI MS m/z 547.2 [M + H]⁺ |
| 1cc | 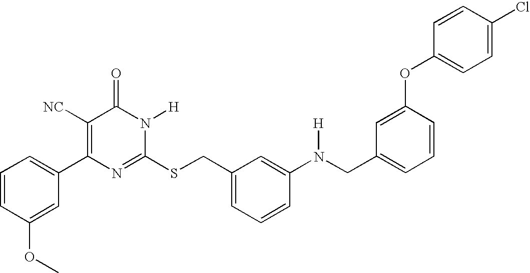 | APCI MS m/z 581.0 [M + H]⁺ |
| 1dd | 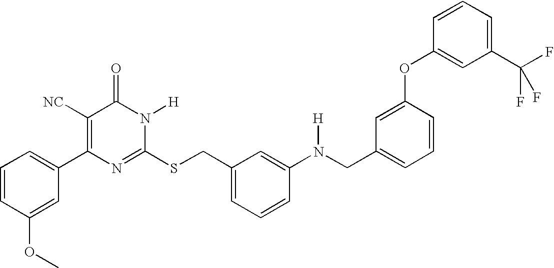 | APCI MS m/z 615.0 [M + H]⁺ |

### Example 1a

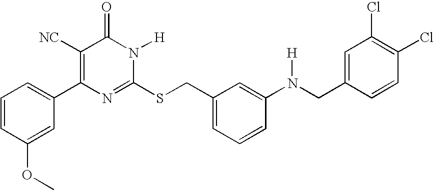

### 2-[3-(3,4-Dichloro-benzylamino)-benzylsulfanyl]-4-(3-methoxy-phenyl)-6-oxo-1,6-dihydro-pyrimidine-5-carbonitrile

Step 1:

Method A:

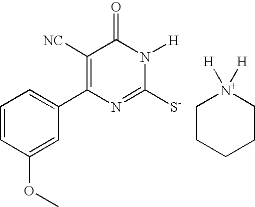

### Mercapto-4-(3-methoxy-phenyl)-6-oxo-1,6-dihydro-pyrimidine-5-carbonitrile, Piperidine Salt

To a round-bottom flask was charged m-anisaldehyde (25.0 mL, 205 mmol), ethyl cyanoacetate (15.6 mL, 205 mmol), thiourea (21.9 mL, 205 mmol), and 200 mL ethanol (EtOH). Piperidine (41 mL, 415 mmol) was added according to the method of Abdou, I. M., et al [Tetrahedron 56: pp. 863-1836 (2000)] and the resulting bright orange solution was heated at reflux for 6 h. After cooling, the solution was left standing until a precipitate formed. The mixture was filtered and the solid washed with ethanol (EtOH). The solids were recombined with EtOH (80 mL) and stirred rapidly, then filtered, washed with EtOH, and dried to provide 24.2 g (34%) of pure product as a light yellow solid. ¹H-NMR (DMSO-d₆) δ 11.60 (bs, 1H), 8.22 (bs, 2H), 7.38 (t, J=8.1 Hz, 1H), 7.26-7.31 (m, 2H), 7.03-7.07 (m, 1H), 3.80 (s, 3H), 3.01 (t, J=5.5 Hz, 4H), 1.54-1.67 (m, 6H).

An alternate method of performing the above 3-component condensation is to use microwave irradiation [See Microwaves in Organic Synthesis, Ed: Loupy, A.; Wiley-VCH, Weinheim, 2002. The reaction can be performed by placing all the reagents including solvent (e.g. EtOH) in a microwave reaction vessel at 100W power for 10-30 minutes. After cooling the reaction vessel to ambient temperature the product can be filtered and washed as above and used directly in the next step.
Method B:

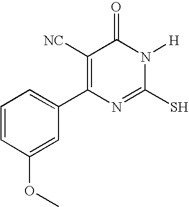

### 6-(3-Methoxy-phenyl)-4-oxo-2-thioxo-1,2,3,4-tetrahydro-pyrimidine-5-carbonitrile

To an Erlenmeyer flask was charged mercapto-4-(3-methoxy-phenyl)-6-oxo-1,6-dihydro-pyrimidine-5-carbonitrile, piperidine salt (10.07 g, 29.2 mmol) and ethyl acetate (EtOAc). 1N HCl was added with rapid mixing until the pH was strongly acidic. The layers were separated and the organics were washed with 1N HCl, then brine, and dried over Na₂SO₄, filtered, concentrated, and dried in a vacuum oven overnight (45° C.) to provide 8.12 g (>100%) pure product (containing some EtOAc) as a white solid. ¹H-NMR (DMSO-d6) δ 13.27 (bs, 1H), 13.18 (s, 1H), 7.44 (t, J=9.0 Hz, 1H), 7.23-7.25 (m, 2H), 7.19 (dd, J=8.1 2.1 Hz, 1H), 3.83 (s, 3H).

Step 2:

### 4-(3-Methoxy-phenyl)-2-(3-nitro-benzylsulfanyl)-6-oxo-1,6-dihydro-pyrimidine-5-carbonitrile

To a round-bottom flask was charged mercapto-4-(3-methoxy-phenyl)-6-oxo-1,6-dihydro-pyrimidine-5-carbonitrile, piperidine salt obtained from Example 1a, Step 1, Method A (9.53 g, 27.7 mmol), m-nitrobenzyl bromide (6.62 g, 30.6 mmol), and 51 mL of dimethylformamide (DMF). The resulting clear, yellow solution was stirred at room temperature overnight. HPLC analysis indicated the reaction was complete. The solution was diluted with hexanes (205 mL) and EtOH (75 mL) and stirred for 2.5 h. A precipitate formed, and the mixture was filtered and washed with 6:1 hexanes:EtOH (70 mL). The solids were recombined with hexanes (51 mL) and EtOH (76 mL) and stirred rapidly for 15 min. The mixture was filtered, the solids were washed with 5:1 hexanes:EtOH (60 mL), then dried to provide 10.21 g (93%) of pure product as a white powder. ¹H-NMR (DMSO-d₆) δ 8.31 (t, J=2.1 Hz, 1H), 8.11 (dd, J=7.5, 1.5 Hz, 1H), 7.89 (d, J=6.9 Hz, 1H), 7.60 (t, J=8.1 Hz, 1H), 7.46-7.48 (m, 2H), 7.37-7.39 (m, 1H), 7.17-7.20 (m, 1H), 4.66 (s, 2H), 3.80 (s, 3H).
Step 3:

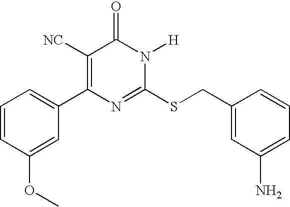

### 2-(3-Amino-benzylsulfanyl)-4-(3-methoxy-phenyl)-6-oxo-1,6-dihydro-pyrimidine-5-carbonitrile

Method A: To a 250 mL round bottom flask was added 4-(3-methoxy-phenyl)-2-(3-nitro-benzylsulfanyl)-6-oxo-1,6-dihydro-pyrimidine-5-carbonitrile (0.95 g, 2.54 mmol) obtained from Example 1a, Step 2, and 20 mL of tetrahydrofuran (THF). Heat was applied to homogenize the solution. After cooling to room temperature, a solution of tin chloride dihydrate (5.73 g, 25.4 mmol) in concentrated HCl (8 mL) was added in dropwise. The mixture was stirred for 1 h and then poured into a cold solution of NaOH (200 mL, 2 N). The solution was extracted with ethyl acetate (3×100 mL), the organic layer washed with brine (200 mL), dried (MgSO₄) and concentrated by rotary evaporation. The residue was filtered through a plug of silica (15 % MeOH/CH₂Cl₂), and the solvent was removed to yield the amine (0.65 g, 17.8 mmol, 70%) as a pale yellow solid: ¹H NMR (300 MHz, DMSO-d6) δ 7.55-8.49 (br, 2H), 7.41-7.55 (m, 4H), 7.15 (dd, 1H), 6.92 (t, 1H), 6.58 (s, 1H), 6.52 (d, 1H), 6.45 (d, 1H), 4.35 (s, 2H), 3.78 (s, 3H); APCI MS m/z 365 [M+H]⁺.

Method B: To a round-bottom flask was charged 4-(3-methoxy-phenyl)-2-(3-nitro-benzylsulfanyl)-6-oxo-1,6-dihydro-pyrimidine-5-carbonitrile, obtained from Example 1a, Step 2 (1.96 g, 4.97 mmol), tetrahydrofuran (46 mL), H₂ O (15 mL), and concentrated HCl (2.7 mL). Over 30 min., indium powder (2.33 g, 20.3 mmoL) was added in portions. The resulting clear solution with suspended gray solids was stirred overnight. HPLC analysis indicated the reaction was complete. The solution was poured into H₂ O (150 mL) and stirred during addition of sat'd., aq. NaHCO₃ until the pH was ˜9. The mixture was filtered and the white filter cake was washed with THF.

The filtrate was saturated with NaCl and extracted with ethyl acetate (EtOAc). The organics were washed with brine, dried over Na₂SO₄, filtered, concentrated, and dried to provide 2.00 g (>100%) crude product as a yellow solid which was used without further purification. ¹H-NMR (DMSO-d₆) δ 7.32-7.39 (m, 3H), 7.01-7.08 (m, 1H), 6.92 (t, J=8.1 Hz, 1H), 6.57 (s, 1H), 6.51 (d, J=7.5 Hz, 1H), 6.42 (dd, J=8.1, 1.8 Hz, 1H), 4.16 (s, 2H), 3.79 (s, 3H).

Step 4:

To a round-bottom flask was charged crude 2-(3-amino-benzylsulfanyl)-4-(3-methoxy-phenyl)-6-oxo-1,6-dihydro-pyrimidine-5-carbonitrile, obtained from Example 1a, Step 3, Method B (theor. 1.81 g, 4.97 mmol), 3,4-dichlorobenzaldehyde (1.04 g, 5.94 mmol), EtOH (50 mL), and MgSO₄ (1.43 g, 11.9 mmol). The mixture was stirred rapidly overnight, then 2 drops of acetic acid (AcOH) and sodium cyanoborohydride (1M in THF, 5.9 mL, 5.9 mmoL) were added. The resulting solution was stirred for 3 h 20 min., then diluted with brine (40 mL) and H₂O (20 mL) and extracted with EtOAc. The organics were washed with brine, dried over Na₂SO₄, filtered, concentrated, and dried to provide 3.5 g of crude product. The crude material was purified by silica gel chromatography (2 consecutive columns at 5% and 2.5% MeOH in dichloromethane, respectively to provide 957 mg (37%) of the title compound as a yellow solid. ¹H-NMR (DMSO-d₆) δ 7.47-7.54 (m, 5H), 7.25 (dd, J=8.1, 1.5 Hz, 1H), 7.15-7.19 (m, 1H), 6.99 (t, J=8.1 Hz, 1H), 6.57-6.62 (m, 2H), 6.45 (dd, J=8.1, 1.2 Hz, 1H), 6.37 (bs, 1H), 4.40 (s, 2H), 4.16 (s, 2H), 3.78 (s, 3H). HRMS (EI⁺) m/z: calc. 523.0762, found 523.0781.

An alternate route for preparing Example 1a is as follows:

Step A:

### 6-(3-Methoxy-phenyl)-4-oxo-2-thioxo-1,2,3,4-tetrahydro-pyrimidine-5-carbonitrile

To an Erlenmeyer flask was charged mercapto-4-(3-methoxy-phenyl)-6-oxo-1,6-dihydro-pyrimidine-5-carbonitrile, piperidine salt (10.07 g, 29.2 mmol), obtained from Example 1a, Step 1, and EtOAc. 1N HCl was added with rapid mixing until the pH was strongly acidic. The layers were separated and the organics were washed with 1N HCl, then brine, and dried over Na₂SO₄, filtered, concentrated, and dried in a vacuum oven overnight (45° C.) to provide 8.12 g (>100%) pure product (containing some EtOAc) as a white solid. ¹H-NMR (DMSO-d₆) δ 13.27 (bs, 1H), 13.18 (s, 1H), 7.44 (t, J=9.0 Hz, 1H), 7.23-7.25 (m, 7.19 (dd, J=8.1, 2.1 Hz, 1H), 3.83 (s, 3H).

Step B:

### [3-(3,4-Dichloro-benzylamino)-phenyl]-methanol

To a round-bottom flask was charged 3-aminobenzyl alcohol (2.50 g, 20.3 mmol), NaHCO₃ (3.41 g, 40.6 mmol), and DMF (30 mL). 3,4-dichlorobenzylchloride (2.80 mL, 20.2 mmol) was added and the mixture was heated at 85° C. for 4 h. HPLC indicated starting materials remained in addition to a major impurity. The solution was diluted with H₂O (300 mL) and extracted with EtOAc. The organics were washed with brine, dried over Na₂SO₄, filtered, concentrated, and dried. The crude material was purified by silica gel chromatography (3% MeOH in dichloromethane) to provide 4.03 g (70%) product in 94% purity (HPLC) as a white solid which upon standing forms an orange oil. ¹H-NMR (DMSO-d₆) δ 7.56-7.59 (m, 2H), 7.33 (dd, J=8.4, 1.5 Hz, 1H), 6.98 (t, J=8.1 Hz, 1H), 6.57 (s, 1H), 6.49 (d, J=7.5 Hz, 1H), 6.39 (dd, J=7.2, 1.5 Hz, 1H), 6.31 (t, J=6.0 Hz, 1H), 4.99 (t, J=5.1 Hz, 1H), 4.34 (d, J=6.0 Hz, 2H), 4.27 (d, J=5.1 Hz, 2H).

Step C:

### 3-Bromomethyl-phenyl)-(3,4-dichloro-benzyl)-amine, hydrobromide Salt

To a round-bottom flask was charged [3-(3,4-dichloro-benzylamino)-phenyl]-methanol (8.15 g, 28.9 mmol) obtained from Example 1a, Step B, and HBr (33 wt % in AcOH, 160 mL). The solution was heated at 85° C. for 1.5 h, then cooled to room temperature. The solution was placed in an ice bath and a white solid precipitated. The solution was diluted with Et₂O (˜200 mL), filtered, and washed with Et₂O. The solids were dried on a vacuum pump to provide 4.32 g (72%) of pure product as a fluffy, white powder. ¹H-NMR (DMSO-d₆) δ 7.57-7.61 (m, 2H), 7.34 (dd, J=8.1, 1.6 Hz, 1H), 7.04 (t, J=7.8 Hz, 1H), 6.79 (s, 1H), 6.63 (d, J=7.8 Hz, 1H), 6.50 (dd, J=8.1, 1.6 Hz, 1H), 5.60 (bs), 4.55 (s, 2H), 4.29 (s, 2H).

Step D:

### 2-[3-(3,4-Dichloro-benzylamino)-benzylsulfanyl]-4-(3-methoxy-phenyl)-6-oxo-1,6-dihydro-pyrimidine-5-carbonitrile

To a round-bottom flask was charged 6-(3-methoxy-phenyl)-4-oxo-2-thioxo-1,2,3,4-tetrahydro-pyrimidine-5-carbonitrile (98 mg, 0.38 mmol) obtained from Example 1a, Step A, 3-bromomethyl-phenyl)-(3,4-dichloro-benzyl)-amine, hydrobromide salt (180 mg, 0.42 mmol) obtained from Example 1a, Step C, and EtOH (3.3 mL). Diisopropylethylamine (0.14 mL, 0.80 mmol) was added dropwise. The resulting clear, yellow solution was stirred at room temperature for 3 h 20 min. HPLC indicated the reaction was complete. The solution was partially concentrated in vacuo and the residue was diluted with saturated aq. NH₄Cl. The mixture was extracted with EtOAc, washed with saturated, aq. NH₄Cl, washed with brine, dried over Na₂SO₄, filtered, concentrated, and dried. The crude material was purified by silica gel chromatography (3% MeOH in dichloromethane) to provide the title compound as a 94% pure product (HPLC).

### Example 1b

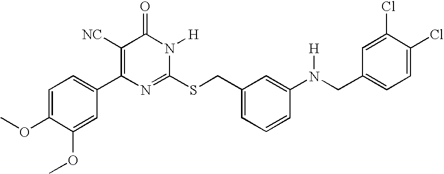

### 2-[3-(3,4-Dichloro-benzylamino)-benzylsulfanyl]-4-(3,4-dimethoxy-phenyl)-6-oxo-1,6-dihydro-pyrimidine-5-carbonitrile

Step 1:

### 4-(3,4-Dimethoxy-phenyl)-2-mercapto-6-oxo-1,6-dihydro-pyrimidine-5-carbonitrile, piperidine Salt

Prepared following the procedure described in Example 1a, Step 1, Method A but with a substitution of the appropriate aldehyde. The crude solid (1.68 g) was purified by slurrying in dichloromethane (15 mL) and MeOH (2 mL), followed by filtration and washing with dichloromethane/MeOH solution, to provide 1.35 g (30%) of pure product as an off-white solid. ¹H-NMR (DMSO-d6) δ 11.95 (bs, 1H), 8.22 (bs, 2H), 7.43 (dd, J=9.1, 2.0 Hz, 1H), 7.38 (d, J=2.0 Hz, 1H), 7.04 (d, J=9.1 Hz, 1H), 3.82 (s, 3H), 3.80 (s, 3H), 3.01 (t, J=5.1 Hz, 4H), 1.54-1.64 (m, 6H).

Step 2:

### 4-(3,4-Dimethoxy-phenyl)-2-(3-nitro-benzylsulfanyl)-6-oxo-1,6-dihydro-pyrimidine-5-carbonitrile

Prepared as in Example 1a, Step 2, with the product obtained from Example 1b, Step 1. The crude material was purified by slurrying in EtOH (40 mL) for 15 min., then filtering and washing with EtOH, to provide 0.87 g (81%) of pure product as a faint yellow solid. ¹H-NMR (DMSO-d₆) δ 8.30-8.34 (m, 1H), 8.11 (dd, J=7.4, 1.2 Hz, 1H), 7.90 (d, J=7.4 Hz, 1H), 7.65 (dd, J=7.4, 1.2 Hz, 1H), 7.62 (d, J=8.3 Hz, 1H), 7.57 (d, J=1.8 H, 1H), 7.14 (d, J=8.3 Hz, 1H), 4.70 (s, 2H), 3.86 (s, 3H), 3.77 (s, 3H).

Step 3:

### 2-(3-Amino-benzylsulfanyl)-4-(3,4-dimethoxy-phenyl)-6-oxo-1,6-dihydro-pyrimidine-5-carbonitrile

Prepared as in Example 1a, Step 3, Method B, with the product obtained from Example 1b, Step 2. The crude material was purified by slurrying in CH₃CN overnight, then filtering and washing with CH₃CN, to provide 189 mg (23%) of pure product as an orange solid. ¹H-NMR (DMSO-d₆) δ 7.45-7.50 (m, 2H), 7.05 (d, J=8.1 Hz, 1H), 6.92 (t, J=7.8 Hz, 1H), 6.57 (d, J=3.0 Hz, 1H), 6.51 (d, J=8.4 Hz, 1H), 6.41 (dd, J=8.4, 1.2 Hz, 1H), 4.19 (s, 2H), 3.81 (s, 3H), 3.77 (s, 3H), 2.07 (s, 2H).

Step 4:

Prepared as in Example 1a, Step 4, with the product obtained from Example 1b, Step 3. The crude material was purified by silica gel chromatography (5% MeOH in dichloromethane) to provide 164 mg (62%) of the title compound as a yellow solid. ¹H-NMR (DMSO-d₆) δ 7.68-7.71 (m, 1H), 7.60 (d, J=2.4 Hz, 1H), 7.50-7.53 (m, 2H), 7.23 (dd, J=8.4, 2.4 Hz, 1H), 7.12 (d, J=9.3 Hz, 1H), 6.99 (t, J=7.5 Hz, 1H), 6.58-6.63 (m, 1H), 6.44-6.47 (m, 1H), 6.36 (bs, 1H), 4.43 (s, 2H), 4.14 (s, 2H), 3.84 (s, 3H), 3.74 (s, 3H). HRMS (EI⁺) m/z: calc. 523.0868, found 523.0852.

### Example 1c

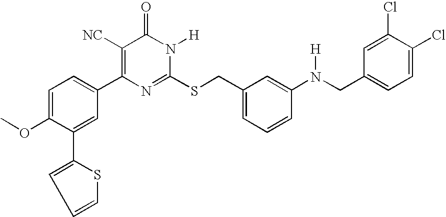

### 2-[3-(3,4-Dichloro-benzylamino)-benzylsulfanyl-4-(4-methoxy-3-thiophen-2-yl-phenyl)-6-oxo-1,6-dihydro-pyrimidine-5-carbonitrile

Step 1:

### 4-Methoxy-3-thiophen-2-yl-benzaldehyde

To a round-bottom flask was charged 3-bromo-4-methoxybenzaldehyde (12.23 g, 56.87 mmol), thiophene-2-boronic acid (8.38 g, 65.5 mmol), ethylene glycol dimethyl ether (310 mL), and 2M aq. Na₂CO₃ (120 mL). The mixture was purged subsurface with nitrogen as tetrakis (triphenylphosphine) palladium(0) (6.58 g, 5.69 mmol) was added. The mixture was purged another 10 min. with nitrogen, then heated at reflux for 5 h. HPLC analysis indicated the reaction was complete. After cooling, the mixture was diluted with H₂O (300 mL) and extracted with EtOAc. The organics were washed with brine, dried over Na₂SO₄, filtered, concentrated, and dried. The crude material was purified by silica gel chromatography (20-25% EtOAc in hexanes) to provide 11.31 g (91%) of pure product as an orange oil. ¹H-NMR (CDCl₃) δ 9.94 (s, 1H), 8.16 (d, J=2.4 Hz, 1H), 7.80 (dd, J=8.8, 2.4 Hz, 1H), 7.57 (dd, J=3.7, 1.5 Hz, 1H), 7.38 (d, J=5.4 Hz, 1H), 7.12 (dd, J=5.4, 3.7 Hz, 1H), 7.09 (d, J=8.8 Hz, 1H) , 4.03 (s, 3H).

Step 2:

### 2-Mercapto-4-(4-methoxy-3-thiophen-2-yl-phenyl)-6-oxo-1,6-dihydro-pyrimidine-5-carbonitrile, piperidine Salt

Prepared as in Example 1a, Step 1, with the product obtained from Example 1c, Step 1. Filtration of the crude reaction mix followed by washing with EtOH provided 3.76 g (36%) of pure product as a beige solid. ¹H-NMR (DMSO-d₆) δ 11.55 (bs, 1H), 8.21 (bs, 2H), 8.13 (d, J=2.4 Hz, 1H), 7.77 (dd, J=9.0, 2.4 Hz, 1H), 7.57-7.59 (m, 2H), 7.24 (d, J=9.0 Hz, 1H), 7.14 (dd, J=5.4 4.2 Hz, 1H), 3.97 (s, 3H), 3.01 (t, J=5.1 Hz, 1H), 1.53-1.63 (m, 6H).

Step 3:

### 4-(4-Methoxy-3-thiophen-2-yl-phenyl)-2-(3-nitro-benzylsulfanyl)-6-oxo-1,6-dihydro-pyrimidine-5-carbonitrile

Prepared as in Example 1a, Step 2, with the product obtained from Example 1c, Step 2. The crude product was purified by slurrying in EtOH (12 mL) and hexanes (2 mL) for 15 min., then filtering and washing with EtOH, to provide 1.08 g (96%) of pure product as a yellow solid. ¹H-NMR (DMSO-d6) δ 8.33-8.34 (m, 1H), 8.30 (d, J=2.1 Hz, 1H), 8.10 (dd, J=7.2, 1.5 Hz, 1H), 7.96 (dd, J=8.1, 2.1 Hz, 1H), 7.90 (d, J=7.5 Hz, 1H), 7.56-7.61 (m, 2H), 7.52 (d, J=3.6 Hz, 1H), 7.33 (d, J=9.0 Hz, 1H), 7.13 (dd, J=5.4, 3.9 Hz, 1H). 4.70 (s, 2H), 4.01 (s, 3H).

Step 4:

### 2-(3-Amino-benzylsulfanyl)-4-(4-methoxy-3-thiophen-2-yl-phenyl)-6-oxo-1,6-dihydro-pyrimidine-5-carbonitrile

Prepared as in Example 1a, Step 3, Method B, with the product obtained from Example 1c, Step 3. The crude material was purified by slurrying in CH₃CN for 1 h, then filtering and washing with CH₃CN, to provide 648 mg (64%) of pure product as an orange-yellow solid. ¹H-NMR (DMSO-d₆) δ 8.16 (d, J=2.4 Hz, 1H), 7.78 (dd, J=8.4, 2.4 Hz, 1H), 7.54-7.58 (m, 2H), 7.23 (d, J=8.7 Hz, 1H), 7.13 (dd, J=4.5, 4.2 Hz, 1H), 6.92 (t, J=7.5 Hz, 1H), 6.58 (s, 1H), 6.51 (d, J=7.5 Hz, 1H), 6.55 (d, J=7.5 Hz, 1H), 5.02 (bs, 2H), 4.15 (s, 2H), 3.96 (s, 3H).

Step 5:

Prepared as in Example 1a, Step 4, with the product obtained from Example 1c, Step 4. The crude material was purified by silica gel chromatography (3% MeOH in dichloromethane) to provide 120 mg (44%) of the title compound as a yellow solid. ¹H-NMR (DMSO-d6) δ 8.38 (d, J=2.1 Hz, 1H), 8.00 (dd, J=7.8, 2.1 Hz, 1H), 7.60 (d, J=5.1 Hz, 1H), 7.52 (dd, J=3.6, 1.5 Hz, 1H), 7.46-7.49 (m, 3H), 7.32 (d, J=9.0 Hz, 1H), 7.20 (dd, J=8.4, 1.5 Hz, 1H), 7.13 (dd, J=5.1, 3.6 Hz, 1H), 7.00 (t, J=7.8 Hz, 1H), 6.65 (s, 1H), 6.60 (d, J=7.2 Hz, 1H), 6.46 (dd, J=8.4, 1.2 Hz, 1H), 6.34 (bs, 1H), 4.44 (s, 2H), 4.12 (s, 2H), 3.99 (s, 3H). HRMS (EI⁺) m/z: calc. 604.0561, found 604.0576.

### Examples 1d-1dd

Examples 1d-1dd were prepared from 2-(3-amino-benzylsulfanyl)-4-(3-methoxy-phenyl)-6-oxo-1,6-dihydro-pyrimidine-5-carbonitrile obtained from Example 1a, Step 3 following the procedure outlined in Example 1a, Steps 4 using the appropriate aldehyde.

### Example 2

### Preparation of Examples 2a through 2ooo

| | | Mass Spec |
|---|---|---|
| Example | structure | Data |
| 2a | 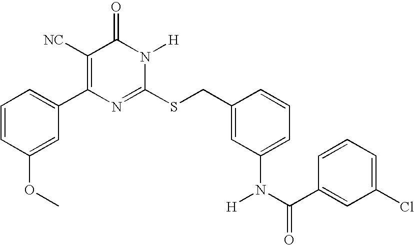 | APCI MS m/z 503.2 [M + H]⁺ |
| 2b | 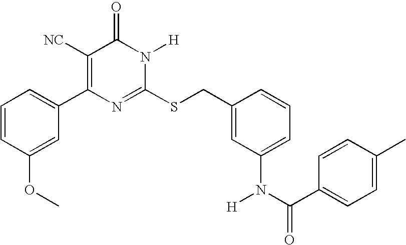 | APCI MS m/z 483.2 [M + H]⁺ |
| 2c | 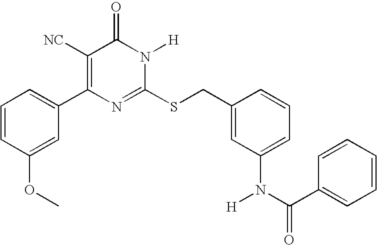 | APCI MS m/z 469.0 [M + H]⁺ |
| 2d | 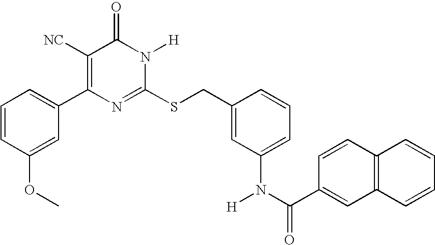 | APCI MS m/z 519.4 [M + H]⁺ |
| 2e | 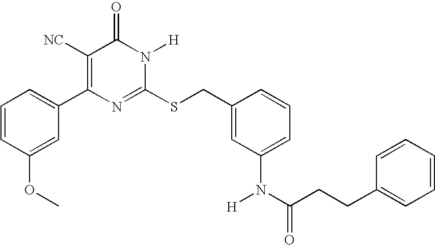 | APCI MS m/z 497.4 [M + H]⁺ |
| 2f | 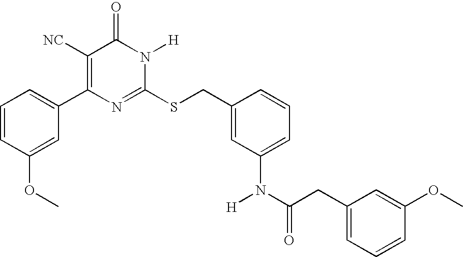 | APCI MS m/z 513.4 [M + H]⁺ |
| 2g | 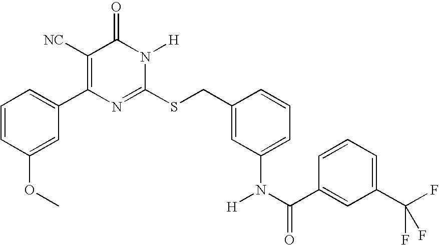 | APCI MS m/z 537.2 [M + H]⁺ |
| 2h | 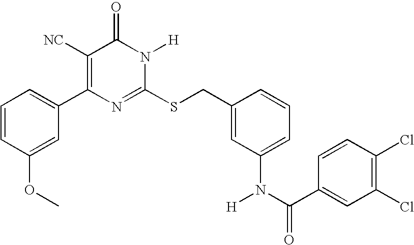 | APCI MS m/z 539.0 [M + H]⁺ |
| 2i | 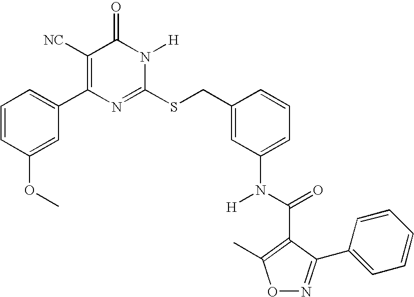 | APCI MS m/z 550.2 [M + H]⁺ |
| 2j | 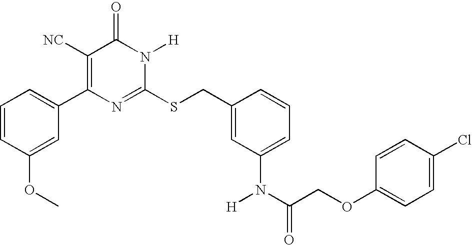 | APCI MS m/z 533.2 [M + H]⁺ |
| 2k | 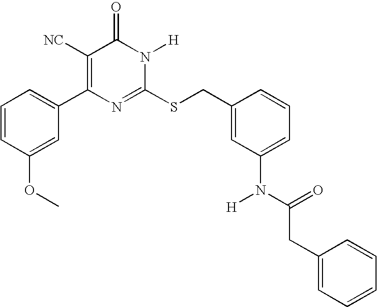 | APCI MS m/z 483.2 [M + H]⁺ |
| 2l | 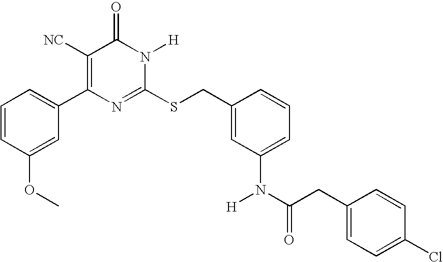 | APCI MS m/z 517.4 [M + H]⁺ |
| 2m | 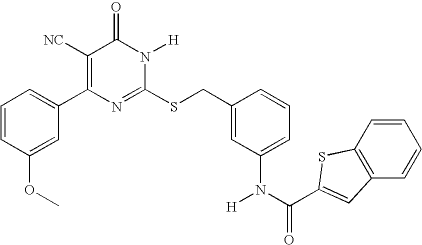 | APCI MS m/z 525.2 [M + H]⁺ |
| 2n | 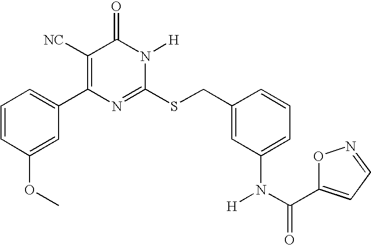 | APCI MS m/z 460.2 [M + H]⁺ |
| 2o | 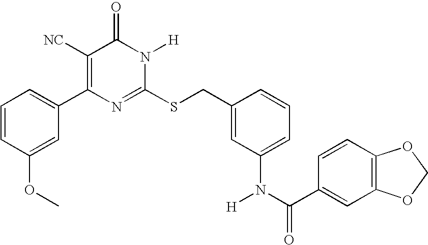 | APCI MS m/z 513.2 [M + H]⁺ |
| 2p | 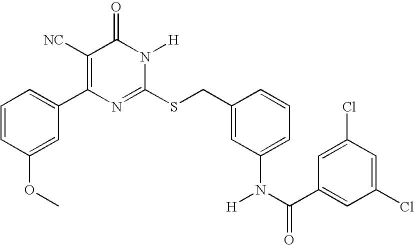 | APCI MS m/z 537.2 [M + H]⁺ |
| 2q | 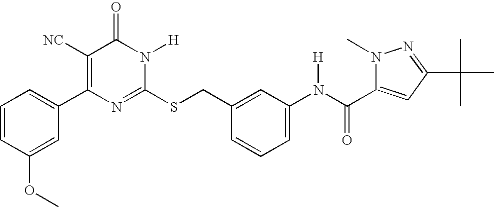 | APCI MS m/z 529.0 [M + H]⁺ |
| 2r | 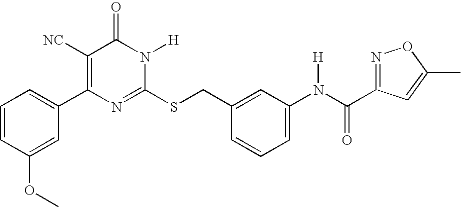 | APCI MS m/z 474.2 [M + H]⁺ |
| 2s | 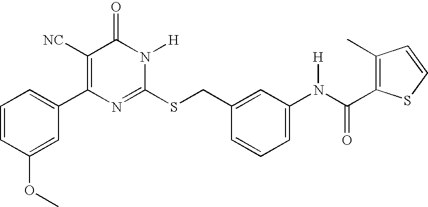 | APCI MS m/z 489.2 [M + H]⁺ |
| 2t | 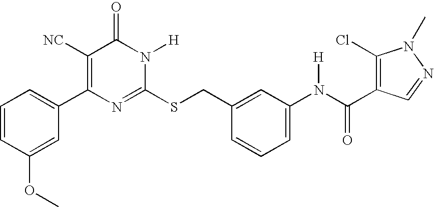 | APCI MS m/z 507.2 [M + H]⁺ |
| 2u | 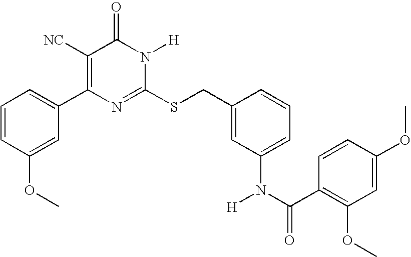 | APCI MS m/z 529.4 [M + H]⁺ |
| 2v | 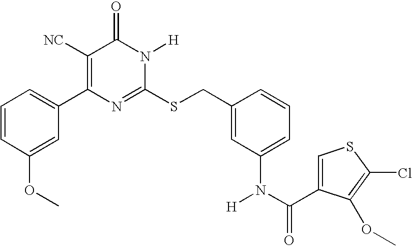 | APCI MS m/z 539.2 [M + H]⁺ |
| 2w | 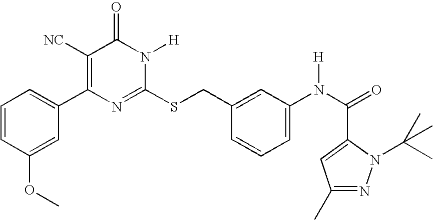 | APCI MS m/z 529.2 [M + H]⁺ |
| 2x | 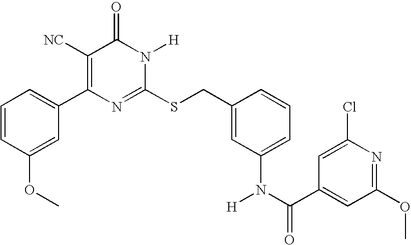 | APCI MS m/z 534.2 [M + H]⁺ |
| 2y | 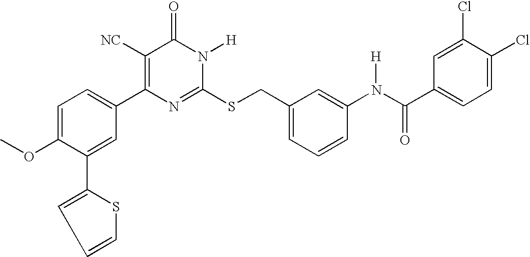 | HRMS (EI⁺) m/z: calc. 619.0432, found 619.0417 |
| 2z | 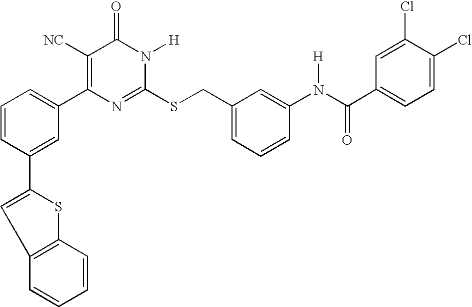 | HRMS (EI⁺) m/z: calc. 639.0483, found 639.0471 |
| 2aa | 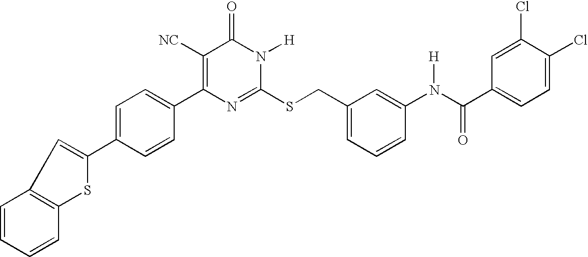 | HRMS (EI⁺) m/z: calc. 639.0483, found 639.0471 |
| 2bb | 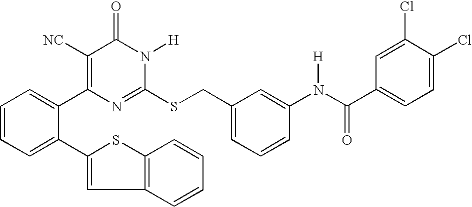 | HRMS (EI⁺) m/z: calc. 639.0483, found 639.0463 |
| 2cc | 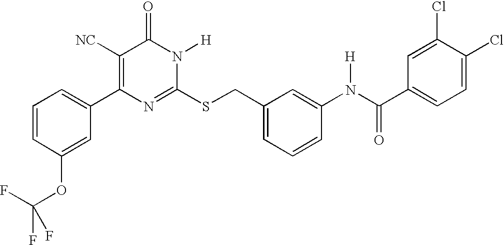 | HRMS (EI⁺) m/z: calc. 591.0272, found 591.0248 |
| 2dd | 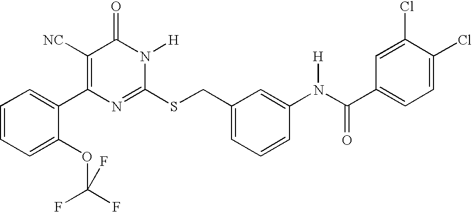 | HRMS (EI⁺) m/z: calc. 591.0272, found 591.0260 |
| 2ee | 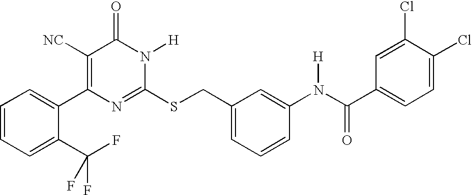 | HRMS (EI⁺) m/z: calc. 575.0323, found 575.0323 |
| 2ff | 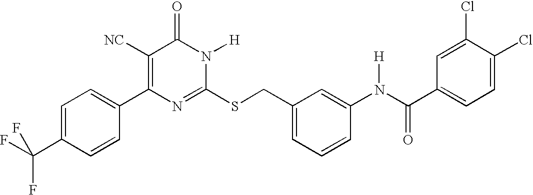 | HRMS (EI⁺) m/z: calc. 575.0323, found 575.0328 |
| 2gg | 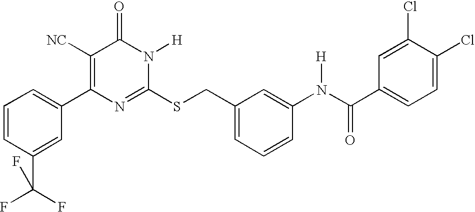 | HRMS (EI⁺) m/z: calc. 575.0323, found 575.0333 |
| 2hh | 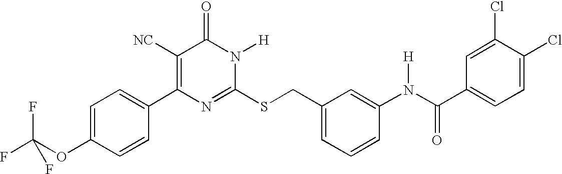 | HRMS (EI⁺) m/z: calc. 591.0272, found 591.0284 |
| 2ii | 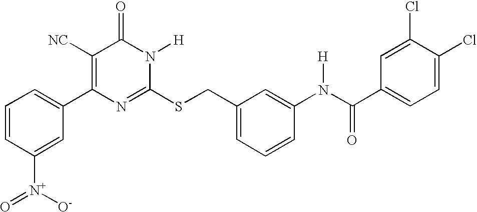 | HRMS (EI⁺) m/z: calc. 552.0300, found 552.0302 |
| 2jj | 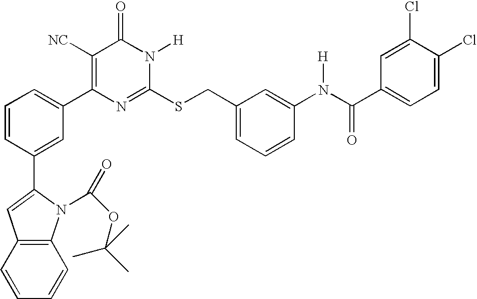 | HRMS (EI⁺) m/z: calc. 722.1395, found 722.1379 |
| 2kk | 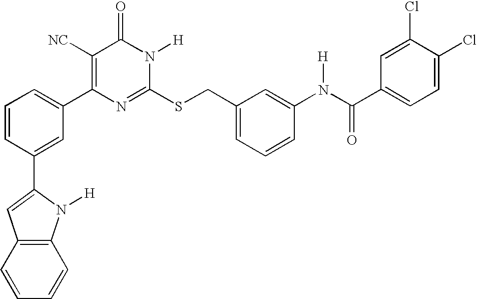 | HRMS (EI⁺) m/z: calc. 622.0871, found 622.0861 |
| 2ll | 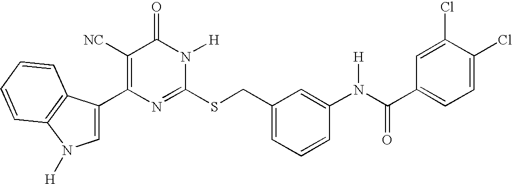 | HRMS (EI⁺) m/z: calc. 545.0480, found 545.0478 |
| 2mm | 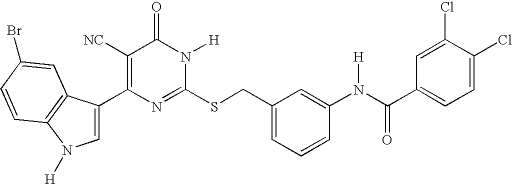 | HRMS (EI⁺) m/z: calc. 623.9485, found 623.9490 |
| 2nn | 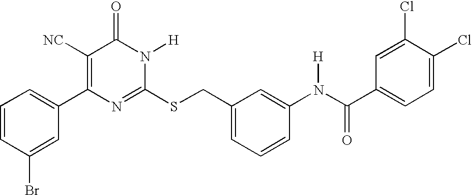 | HRMS (EI⁺) m/z: calc. 584.9554, found 584.9551 |
| 2oo | 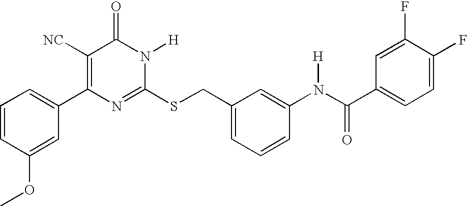 | HRMS (EI⁺) m/z: calc. 623.9485, found 623.9490 |
| 2pp | 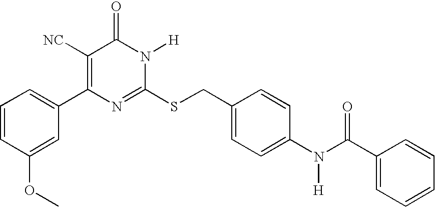 | APCI MS m/z 467.2 [M − H]⁻ |
| 2qq | 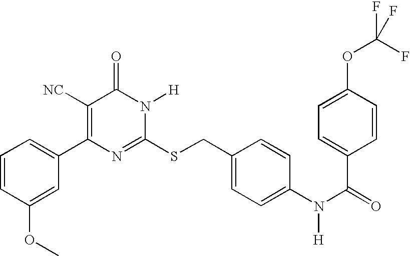 | APCI MS m/z 551.4 [M − H]⁻ |
| 2rr | 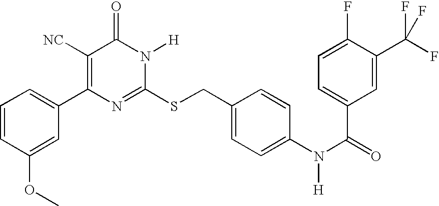 | APCI MS m/z 553.2 [M − H]⁻ |
| 2ss | 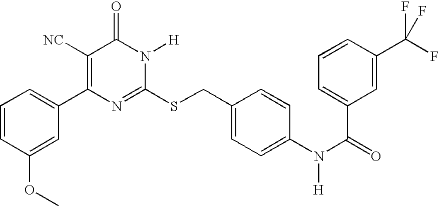 | APCI MS m/z 535.2 [M − H]⁻ |
| 2tt | 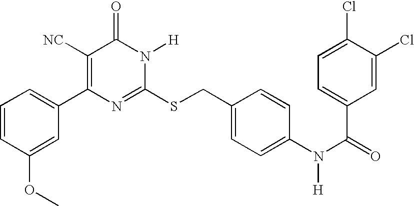 | APCI MS m/z 535.0 [M − H]⁻ |
| 2uu | 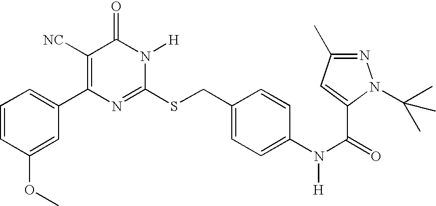 | APCI MS m/z 527.2 [M − H]⁻ |
| 2vv | 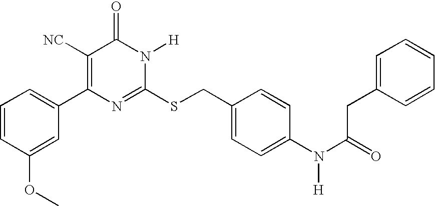 | APCI MS m/z 481.0 [M − H]⁻ |
| 2ww | 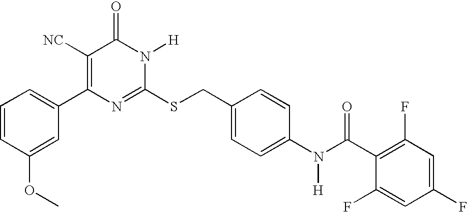 | APCI MS m/z 521.2 [M − H]⁻ |
| 2xx | 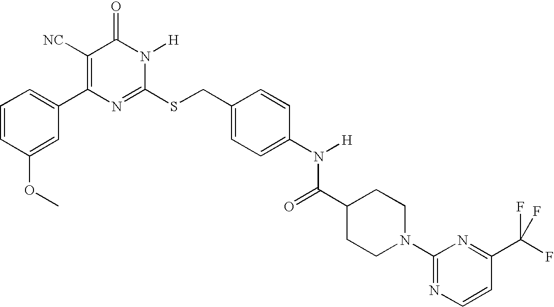 | APCI MS m/z 620.2 [M − H]⁻ |
| 2yy | 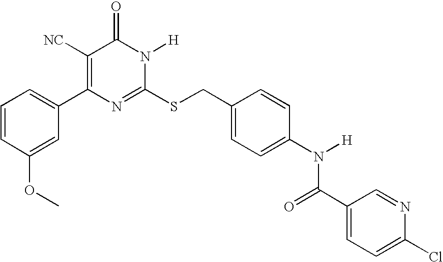 | APCI MS m/z 502.0 [M − H]⁻ |
| 2zz | 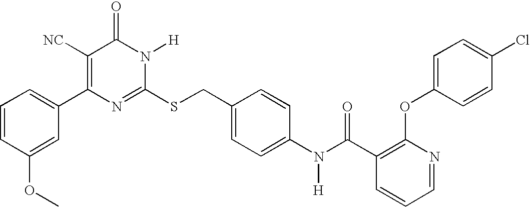 | APCI MS m/z 594.0 [M − H]⁻ |
| 2aaa | 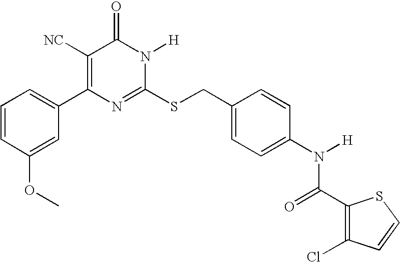 | APCI MS m/z 507.0 [M − H]⁻ |
| 2bbb | 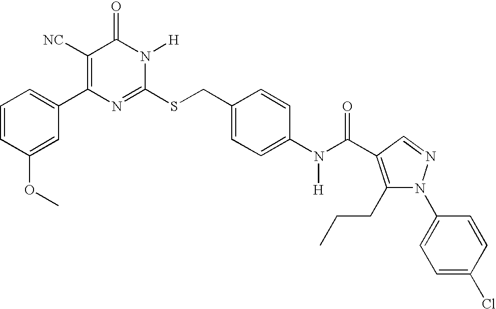 | APCI MS m/z 609.2 [M − H]⁻ |
| 2ccc | 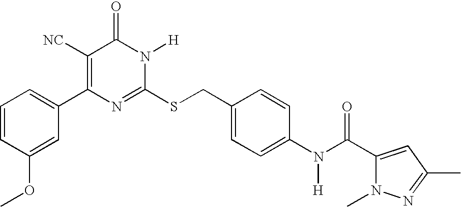 | APCI MS m/z 485.2 [M − H]⁻ |
| 2ddd | 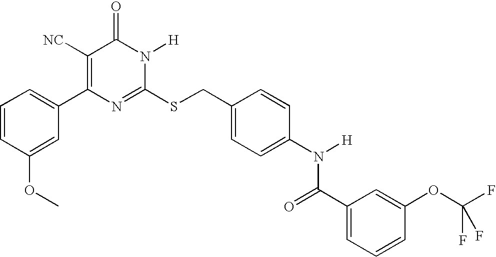 | APCI MS m/z 551.4 [M − H]⁻ |
| 2eee | 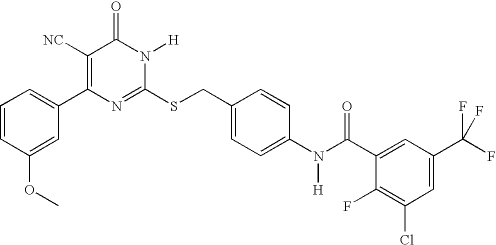 | APCI MS m/z 587.0 [M − H]⁻ |
| 2fff | 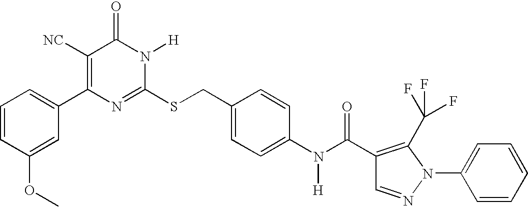 | APCI MS m/z 601.2 [M − H]⁻ |
| 2ggg | 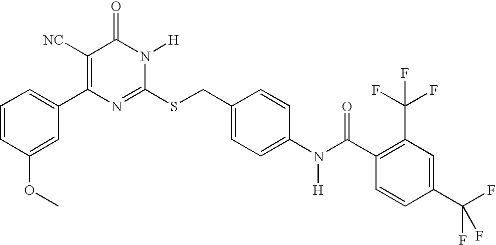 | APCI MS m/z 602.8 [M − H]⁻ |
| 2hhh | 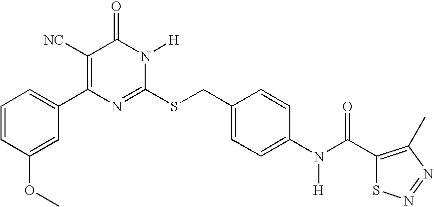 | APCI MS m/z 489.2 [M − H]⁻ |
| 2iii | 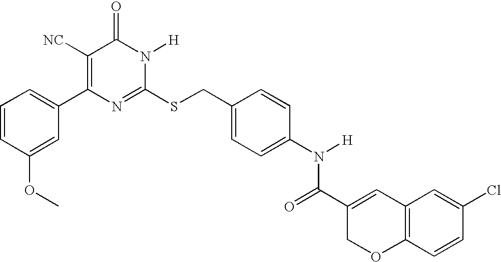 | APCI MS m/z 555.2 [M − H]⁻ |
| 2jjj | 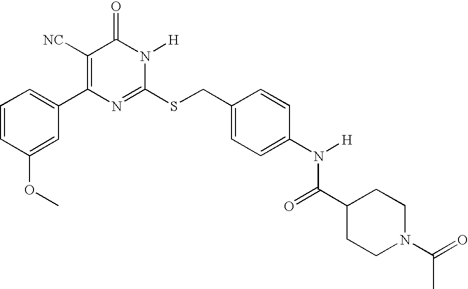 | APCI MS m/z 516.2 [M − H]⁻ |
| 2kkk | 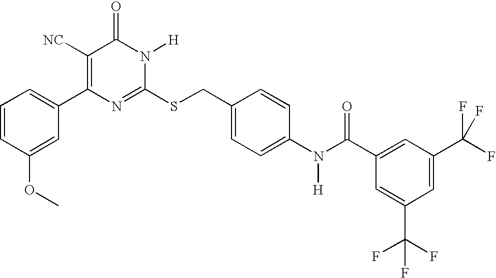 | APCI MS m/z 603.4 [M − H]⁻ |
| 2lll | 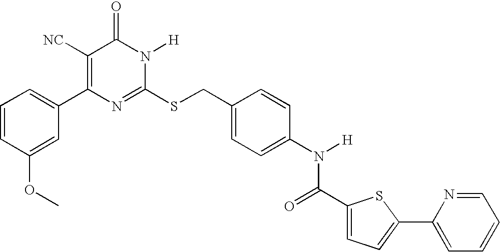 | APCI MS m/z 550.2 [M − H]⁻ |
| 2mmm | 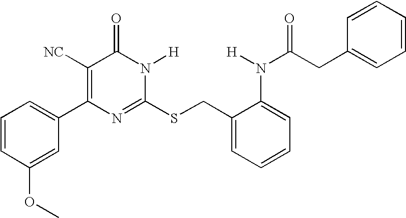 | APCI MS m/z 483.2 [M + H]⁺ |
| 2nnn | 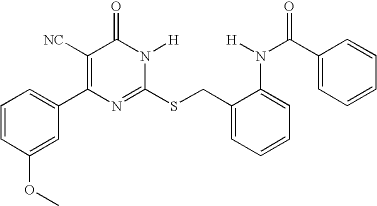 | APCI MS m/z 469.0 [M + H]⁺ |
| 2ooo | 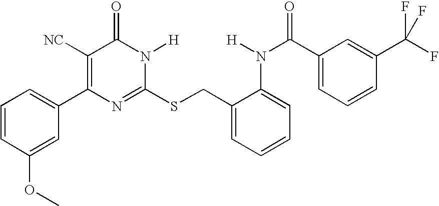 | APCI MS m/z 537.0 [M + H]⁺ |

### Example 2a

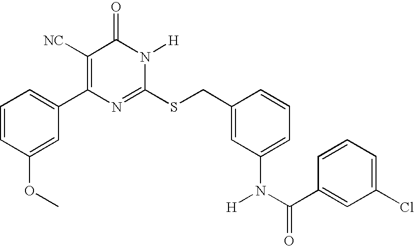

### 3-Chloro-N-{3-[5-cyano-4-(3-methoxy-phenyl)-6-oxo-1,6-dihydro-pyrimidin-2-ylsulfanylmethyl]-phenyl}-benzamide

To a round-bottom flask was added 2-(3-amino-benzylsulfanyl)-4-(3-methoxy-phenyl)-6-oxo-1,6-dihydro-pyrimidine-5-carbonitrile (0.21 g, 0.58 mmol), obtained from Example 1a, Step 3, polyvinylpyridine/10% (w/w) polymer-supported dimethylaminopyridine (0.16 g), and THF (5 mL). To the stirring mixture was added 3-chlorobenzoyl chloride (0.1 12 g, 0.64 mmol), and the flask was capped and stirred for 18 h at ambient temperature. The polymer support was then filtered, and the solvent concentrated by rotary evaporation. The residue was purified by trituration with CH₃CN/MeOH (10:1) to yield the title compound (0.041 g, 0.28 mmol, 14%) as a white solid: ¹H-NMR (300 MHz, DMSO-d₆) d 10.39 (s, 1H), 7.99 (m, 1H), 7.89 (m, 2H), 7.41-7.71 (m, 7H), 7.31 (t, 1H), 7.19 (m, 2 H), 4.56 (s, 2H), 3.78 (s, 3H); LC-MS 98.7% pure (AUC), tR=4.46 min; APCI MS m/z 503.2 [M+H]⁺.

### Examples 2b -2x

Examples 2b -2x were prepared from 2-(3-amino-benzylsulfanyl)-4-(3-methoxy-phenyl)-6-oxo-1,6-dihydro-pyrimidine-5-carbonitrile obtained from Example 1a, Step 3 following the procedures outlined in Example 2a using the appropriate functionalized carbonyl derivative as a reagent.

### Example 2y

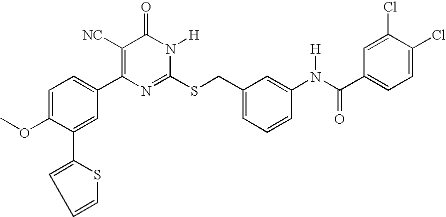

### 3,4-Dichloro-N-{3-[5-cyano-4-(4-methoxy-3-thiophen-2-yl-phenyl)-6-oxo-1,6-dihydro-pyrimidin-2-ylsulfanylmethyl]-phenyl}-benzamide

Step 1:

### 3,4-Dichloro-N-(3-hydroxymethyl-phenyl)-benzamide

To a round-bottom flask was charged 3-aminobenzyl alcohol (3.96 g, 32.2 mmol), dioxane (32 mL), and triethylamine (5.0 mL, 36 mmol). As solution of 3,4-dichlorobenzoylchloride (6.76 g, 32.3 mmol) in dioxane (32 mL) was added via addition funnel over 30 min. A gray precipitate formed during addition. After stirring an additional 2 h, the mixture was diluted with H₂O (300 mL) and acidified to pH 1 with 1N HCl (˜15 mL). The mixture was stirred rapidly, filtered, and the solids washed with H₂O. The solids were dried in a vacuum oven (40° C.) overnight to provide 8.94 g (94%) of pure product as a beige solid. ¹H-NMR (DMSO-d₆) δ 10.37 (s, 1H), 8.23 (d, J=2.0 Hz, 1H), 7.94 (dd, J=9.1, 2.0 Hz, 1H), 7.82 (d, J=9.1 Hz, 1H), 7.74 (s, 1H), 7.65 (d, J=8.0 Hz, 1H), 7.31 (t, J=8.0 Hz, 1H), 7.06 (d, J=8.0 Hz, 1H), 5.23 (t, J=5.6 Hz, 1H), 4.50 (d, J=5.6 Hz, 2H).
Step 2:

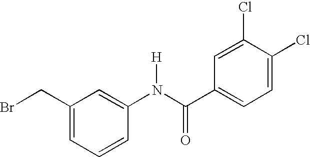

### N-(3-Bromomethyl-phenyl)-3,4-dichloro-benzamide

To a round-bottom flask was charged 3,4-dichloro-N-(3-hydroxymethyl-phenyl)-benzamide, obtained from Example 2y, Step 1 (5.13 g, 17.3 mmol) and HBr (33 wt % in AcOH, 63 mL). The reaction was heated at 90° C. for 10 min., and then cooled to room temperature. The solution was placed in an ice bath and diluted with ice and H₂O and a white solid precipitated. The mixture was filtered and the solids were washed with H₂O. The solids were dried in a vacuum oven (40° C.) overnight to provide 5.83 g (94%) of pure product as a gray solid. ¹H-NMR (DMSO-d₆) δ 10.46 (s, 1H), 8.23 (d, J=2.1 Hz, 1H), 7.94 (dd, J=8.5, 2.1 Hz, 1H), 7.89 (s, 1H), 7.83 (d, J=8.5 Hz, 1H), 7.68 (d, J=8.1 Hz, 1H), 7.36 (t, J=8.1 Hz, 1H), 7.21 (d, J=8.1 Hz, 1H), 4.72 (s, 2H).

Step 3:

To a round-bottom flask was charged 2-mercapto-4-(4-methoxy-3-thiophen-2-yl-phenyl)-6-oxo-1,6-dihydro-pyrimidine-5-carbonitrile piperidine salt, prepared in Example 1c Steps 1-2, (54 mg, 0.13 mmol), N-(3-bromomethyl-phenyl)-3,4-dichloro-benzamide, prepared in Example 2y, Step 2 (55 mg, 0.15 mmol), and DMF (0.6 mL). The solution was stirred overnight. HPLC analysis indicated the reaction was complete. The solution was diluted with H₂O and extracted with EtOAc. The organics were washed with brine, dried over Na₂SO₄, filtered, concentrated, and dried. The crude material was purified by silica gel chromatography (0-5% MeOH in dichloromethane), followed by slurrying in EtOH (12 mL), to provide 41 mg (52%) of the title compound as a yellow solid. ¹H-NMR (DMSO-d6) δ 10.40 (s, 1H), 8.36 (d, J=1.5 Hz, 1H), 8.18 (d, J=1.5 Hz, 1H), 7.97-8.00 (m, 1H), 7.89-7.92 (m, 2H), 7.81 (d, J=8.7 Hz, 1H), 7.64 (d, J=8.7 Hz, 1H), 7.59 (d, J=4.5 Hz, 1H), 7.28-7.34 (m, 2H), 7.20 (d, J=8.4 Hz, 1H), 7.13 (t, J 4.5 Hz, 1H), 4.59 (s, 2H), 3.98 (s, 3H). HRMS (EI⁺) m/z: calc. 619.0432, found 619.0417.

### Example 2z

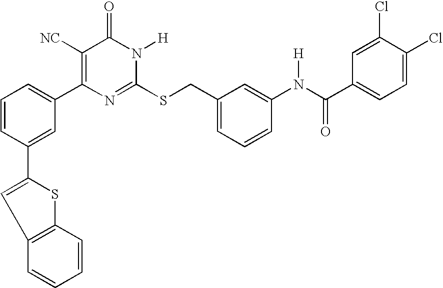

### N-{3-[4-(3-Benzo[b]thiophen-2-yl-phenyl)-5-cyano-6-oxo-1,6-dihydro-pyrimidin-2-ylsulfanylmethyl]-phenyl}-3,4-dichloro-benzamide

Step 1:

### 3-Benzo[b]thiophen-2-yl-benzaldehyde

Prepared following the procedure described in Example 1c Step 1 with the appropriate aldehyde and boronic acid. The crude product was purified by silica gel chromatography (10% EtOAc in hexanes) to provide 1.12 g (87%) of pure product as a yellow solid. ¹H-NMR (CDCl₃) δ 10.10 (s, 1H), 8.22 (d, J=0.9 Hz, 1H), 7.97 (dd, J=8.4, 1.5 Hz, 1H), 7.80-7.87 (m, 3H), 7.66 (s, 1H), 7.61 (t, J=6.6 Hz, 1H), 7.33-7.41 (m, 2H).

Step 2:

### 4-(3-Benzo[b]thiophen-2-yl-phenyl)-2-mercapto-6-oxo-1,6-dihydro-pyrimidine-5-carbonitrile, piperidine Salt

Prepared following the procedure described in Example 1a, Step 1, Method A using the aldehyde prepared in Example 2z Step 1. Filtration of the crude reaction mixture followed by washing with EtOH provided 769 mg (37%) of pure product as a light yellow solid. ¹H-NMR (DMSO-d₆) δ 11.68 (bs, 1H), 8.22 (bs, 2H), 8.10 (t, J=1.8 Hz, 1H), 8.00 (dd, J=7.2, 1.8 Hz, 1H), 7.88-7.91 (m, 3H), 7.75 (d, J=7.8 Hz, 1H), 7.59 (t, J=8.1 Hz, 1H), 7.35-7.44 (m, 2H), 3.01 (t, J=5.4 Hz, 4H), 1.49-1.67 (m, 6H).

Step 3:

### N-{3-[4-(3-Benzo[b]thiophen-2-yl-phenyl)-5-cyano-6-oxo-1,6-dihydro-pyrimidin-2-ylsulfanylmethyl]-phenyl}-3,4-dichloro-benzamide

Prepared following the procedure described in Example 2y, Step 3, using the piperidine salt product from Example 2z, Step 2. The crude material was purified by slurrying in EtOH, followed by slurrying in dichloromethane/MeOH to provide 13 mg (9%) of pure product as a white solid. ¹H-NMR (DMSO-d₆) δ 10.40 (s, 1H), 8.29 (s, 1H), 8.15 (d, J=1.8 Hz, 1H), 7.84-8.02 (m, 7H), 7.77 (d, J=9.0 Hz, 1H), 7.64-7.70 (m, 2H), 7.36-7.42 (m, 2H), 7.32 (t, J=7.5 Hz, 1H), 7.21 (d, J=7.5 Hz, 1H), 4.60 (s, 2H). HRMS (EI⁺) m/z: calc. 639.0483, found 639.0471.

### Example 2aa

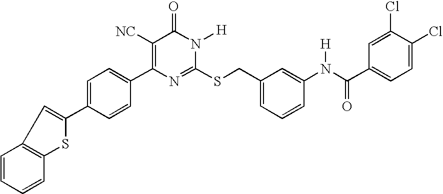

### N-{3-[4-(4-Benzo[b]thiophen-2-yl-phenyl)-5-cyano-6-oxo-1,6-dihydro-pyrimidin-2-ylsulfanylmethyl]-phenyl}-3,4-dichloro-benzamide

Step 1:

### 4-Benzo[b]thiophen-2-yl-benzaldehyde

Prepared following the procedure described in Example 1c Step 1 with the appropriate aldehyde and boronic acid. The crude product was purified by silica gel chromatography (15% EtOAc in hexanes) to provide 1.17 g (91%) of pure product as an orange solid. ¹H-NMR (CDCl₃) δ 10.05 (s, 1H), 7.95 (d, J=8.9 Hz, 2H), 7.88 (d, J=8.9 Hz, 2H), 7.81-7.89 (m, 2H), 7.71 (s, 1H), 7.36-7.40 (m, 2H).

Step 2:

### 4-(4-Benzo[b]thiophen-2-yl-phenyl)-2-mercapto-6-oxo-1,6-dihydro-pyrimidine-5-carbonitrile, piperidine Salt

Prepared following the procedure described in Example 1a, Step 1, Method A using the aldehyde prepared in Example 2aa Step 1. Filtration of the crude reaction mix followed by washing with EtOH provided crude product which was further purified by slurrying in dichloromethane (6 mL) and MeOH (1 mL). After 2 h, the mixture was filtered and the solids washed with a solution of dichloromethane/MeOH to provide 771 mg (35%) of semi-pure product as a light yellow-brown solid. ¹H-NMR (DMSO-d₆) δ 8.02 (d, J=6.6 Hz, 2H), 7.86-7.92 (m, 5H), 7.35-7.45 (m, 2H), 2.98 (t, J=5.3 Hz, 4H), 1.55-1.63 (m, 6H).

Step 3:

Prepared following the procedure described in Example 2y, Step 3, using the piperidine salt product from Example 2aa, Step 2. The crude reaction mixture was diluted with EtOAc (5 mL) and stirred rapidly, then filtered. The resulting solids were slurried in EtOAc (10 mL) for 1h 10 min., then filtered and washed with EtOAc, to provide 102 mg (60%) of the title compound. ¹H-NMR (DMSO-d6) δ 10.42 (s, 1H), 8.21 (d, J=2.1 Hz, 1H), 7.92-8.05 (m, 8H), 7.86-7.89 (m, 1H), 7.77 (d, J=7.5 Hz, 1H), 7.59 (d, J=9.0 Hz, 1H), 7.39-7.46 (m, 2H), 7.35 (t, J=8.1 Hz, 1H), 7.23 (d, J=8.4 Hz, 1H), 4.56 (s, 2H). HRMS (EI⁺) m/z: calc. 639.0483, found 639.0427.

### Example 2bb

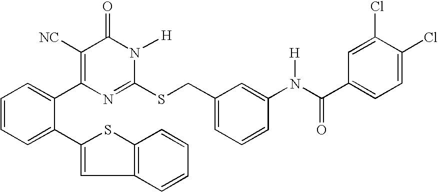

### N-{3-[4-(2-Benzo[b]thiophen-2-yl-phenyl)-5-cyano-6-oxo-1,6-dihydro-pyrimidin-2-ylsulfanylmethyl]-phenyl}-3,4-dichloro-benzamide

Step 1:

### 2-Benzo[b]thiophen-2-yl-benzaldehyde

Prepared following the procedure described in Example 1c Step 1 with the appropriate aldehyde and boronic acid. The crude product was purified by silica gel chromatography (10% EtOAc in hexanes) to provide 544 mg (42%) of pure product as an orange oil. ¹H-NMR (CDCl₃) δ 10.26 (s, 1H), 8.05 (d, J=8.1 Hz, 1H), 7.82-7.90 (m, 2H), 7.63-7.70 (m, 2H), 7.52-7.57 (m, 1H), 7.37-7.46 (m, 2H), 7.28 (s, 1H).

Step 2:

### 4-(2-Benzo[b]thiophen-2-yl-phenyl)-2-mercapto-6-oxo-1,6-dihydro-pyrimidine-5-carbonitrile, piperidine Salt

Prepared following the procedure described in Example 1a, Step 1, Method A using the aldehyde prepared in Example 2bb Step 1. The crude reaction mixture was partially concentrated in vacuo. Filtration of the concentrated mix followed by washing with EtOH provided 275 mg (27%) of semi-pure product as a yellow, semi-crystalline solid. ¹H-NMR (DMSO-d₆) δ 11.60 (bs, 1H), 8.27 (bs, 2H), 7.93-7.98 (m, 1H), 7.81-7.84 (m, 1H), 7.70 (dd, J=7.8, 1.2 Hz, 1H), 7.47-7.58 (m, 2H), 7.30-7.41 (m, 4H), 2.98 (t, J=5.4 Hz, 4H), 1.53-16.2 (m, 6H).

Step 3:

Prepared following the procedure described in Example 2y, Step 3, using the piperidine salt product from Example 2bb, Step 2. The crude material was purified by silica gel chromatography (5% MeOH in dichloromethane) to provide 103 mg (66%) of the title compound. ¹H-NMR (DMSO-d₆) δ 10.33 (s, 1H), 8.16 (d, J=2.1 Hz, 1H), 7.91-7.96 (m, 1H), 7.88 (d, J=2.7 Hz, 1H), 7.80-7.83 (m, 2H), 7.73 (d, J=7.5 Hz, 1H), 7.53-7.66 (m, 5H), 7.33-7.28 (m, 3H), 7.09 (t, J=7.2 Hz, 1H), 6.77 (bs, 1H), 4.15 (s, 2H). HRMS (EI⁺) m/z: calc. 639.0483, found 639.0463.

### Example 2cc

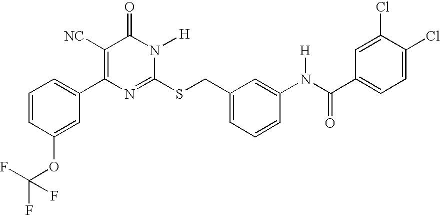

### 3,4-Dichloro-N-{3-[5-cyano-6-oxo-4-(3-trifluoromethoxy-phenyl)-1,6-dihydro-pyrimidin-2-ylsulfanylmethyl]-phenyl}-benzamide

Step 1:

### 2-Mercapto-6-oxo-4-(3-trifluoromethoxy-phenyl)-1,6-dihydro-pyrimidine-5-carbonitrile, piperidine Salt

Prepared following the procedure described in Example 1a, Step 1, Method A with the appropriate aldehyde. The crude reaction mixture was diluted with brine and H₂O and extracted with EtOAc. The organics were washed with brine, dried over Na₂SO₄, filtered, concentrated, and dried. The crude material was purified by slurrying in EtOAc (10 mL) and hexanes (17 mL) to provide 519 mg (<25%) of semi-pure product (which contained unreacted thiourea) as a light yellow solid. The material was used in the subsequent reaction without further purification. ¹H-NMR (DMSO-d₆) δ 11.72 (bs, 1H), 8.24 (bs, 2H), 7.79 (d, J=7.8 Hz, 1H), 7.66 (s, 1H), 7.62 (t, J=8.4 Hz, 1H), 7.50 (d, J=8.1 Hz, 1H), 3.01 (t, J=5.4 Hz, 4H), 1.54-1.67 (m, 6H).

Step 2:

Prepared following the procedure described in Example 2y, Step 3, using the piperidine salt product from Example 2cc, Step 1. The crude material was purified by silica gel chromatography (5% MeOH in dichloromethane) to provide 92 mg (59%) of the title compound as a white solid. ¹H-NMR (DMSO-d₆) δ 10.40 (s, 1H), 8.19 (d, J=2.4 Hz, 1H), 7.90-7.95 (m, 2H), 7.80-7.87 (m, 3H), 7.69 (t, J=8.1 Hz, 1H), 7.57-7.64 (m, 2H), 7.30 (t, J=7.2 Hz, 1H), 7.17 (d, J=7.8 Hz, 1H), 4.50 (s, 2H). HRMS (EI⁺) m/z: calc. 591.0272, found 591.0248.

### Example 2dd

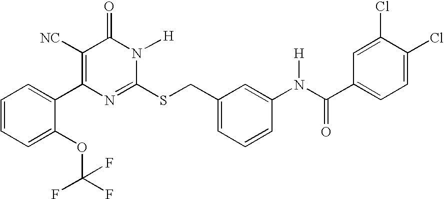

### 3,4-Dichloro-N-{3-[5-cyano-6-oxo-4-(2-trifluoromethoxy-phenyl)-1,6-dihydro-pyrimidin-2-ylsulfanylmethyl]-phenyl}-benzamide

Step 1:

### 2-Mercapto-6-oxo-4-(2-trifluoromethoxy-phenyl)-1,6-dihydro-pyrimidine-5-carbonitrile, piperidine Salt

Prepared following the procedure described in Example 1a, Step 1, Method A with the appropriate aldehyde. Filtration of the crude reaction mixture followed by washing with EtOH provided 608 mg (27%) of pure product as a white solid. ¹H-NMR (DMSO-d₆) δ 11.67 (bs, 1H), 8.22 (bs, 2H), 7.54-7.61 (m, 1H), 7.43-7.50 (m, 3H), 3.00 (t, J=5.1 Hz, 4H), 1.54-1.67 (m, 6H).

Step 2:

Prepared following the procedure described in Example 2y, Step 3, using the piperidine salt product from Example 2dd, Step 1. The crude material was purified by silica gel chromatography (7% MeOH in dichloromethane) to provide 113 mg (82%) of the title compound as a white foam. ¹H-NMR (DMSO-d6) δ 10.38 (s, 1H), 8.20 (d, J=3.0 Hz, 1H), 7.92 (dd, J=9.0, 2.4 Hz, 1H), 7.83 (d, J=8.4 Hz, 1H), 7.76 (s, 1H), 7.61-7.67 (m, 3H), 7.50-7.55 (m, 2H), 7.28 (t, J=7.2 Hz, 1H), 7.12 (d, J=7.2 Hz, 1H), 7.12 (d, J=7.2 Hz, 1H), 4.38 (s, 1H). HRMS (EI⁺) m/z: calc. 591.0272, found 591.0260.

### Example 2ee

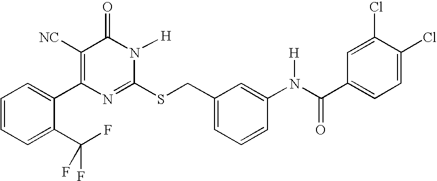

### 3,4-Dichloro-N-{3-[5-cyano-6-oxo-4-(2-trifluoromethyl-phenyl)-1,6-dihydro-pyrimidin-2-ylsulfanylmethyl]-phenyl}-benzamide

Step 1:

### 2-Mercapto-6-oxo-4-(2-trifluoromethyl-phenyl)-1,6-dihydro-pyrimidine-5-carbonitrile, piperidine Salt

Prepared following the procedure described in Example 1a, Step 1, Method A with the appropriate aldehyde. Filtration of the crude reaction mixture followed by washing with EtOH provided 221 mg (10%) of pure product as an off-white solid. ¹H-NMR (DMSO-d₆) δ 11.71 (bs, 1H), 8.22 (bs, 2H), 7.80 (d, J=7.5 Hz, 1H), 7.72-7.77 (m, 1H), 7.63-7.68 (m, 1H), 7.43 (d, J=8.4 Hz, 1H), 3.01 (t, J=4.8 Hz, 4H), 1.54-1.64 (m, 6H).

Step 2:

Prepared following the procedure described in Example 2y, Step 3, using the piperidine salt product from Example 2ee, Step 1. The crude material was purified by silica gel chromatography (7.5% MeOH in dichloromethane) to provide 93 mg (91%) of the title compound as a white foam. ¹H-NMR (DMSO-d6) δ 10.37 (s, 1H), 8.20 (d, J=1.5 Hz, 1H), 7.73-7.95 (m, 6H), 7.58-7.65 (m, 2H), 7.27 (t, J=7.2 Hz, 1H), 7.09 (d, J=7.8 Hz, 1H), 4.34 (s, 2H). HRMS (EI⁺) m/z: calc. 575.0323, found 575.0323.

### Example 2ff

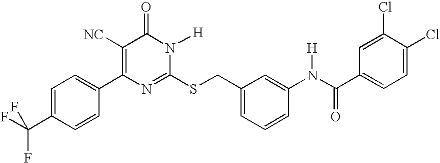

### 3,4-Dichloro-N-{3-[5-cyano-6-oxo-4-(4-trifluoromethyl-phenyl)-1,6-dihydro-pyrimidin-2-ylsulfanylmethyl]-phenyl}-benzamide

Step 1:

### 2-Mercapto-6-oxo-4-(4-trifluoromethyl-phenyl)-1,6-dihydro-pyrimidine-5-carbonitrile, piperidine Salt

Prepared following the procedure described in Example 1a, Step 1, Method A with the appropriate aldehyde. The crude reaction mixture was concentrated in vacuo, then diluted with H₂O, saturated with NaCl, and extracted with EtOAc. The organic phase mixture was washed with brine, then filtered. The solids were washed with EtOAc to provide 293 mg (14%) of pure product as a light yellow solid. ¹H-NMR (DMSO-d₆) δ 11.72 (bs, 1H), 8.44 (bs, 2H), 7.92 (d, J=8.5 Hz, 2H), 7.84 (d, J=8.5 Hz, 2H), 2.99 (t, J=5.1 Hz, 4H), 1.54-1.68 (m, 6H).

Step 2:

Prepared following the procedure described in Example 2y, Step 3, using the piperidine salt product from Example 2ff, Step 1. The crude material was purified by silica gel chromatography (10% MeOH in dichloromethane) to provide 54 mg (73%) of the title compound as an off-white solid. ¹H-NMR (DMSO-d₆) δ 10.40 (s, 1H), 8.21 (d, J=1.8 Hz, 1H), 7.98 (d, J=8.5 Hz, 2H), 7.93 (dd, J=7.8, 1.8 Hz, 1H), 7.85 (d, J=8.5 Hz, 2H), 7.81 (d, J=8.7 Hz, 2H), 7.65 (d, J=7.2 Hz, 1H), 7.31 (t, J=7.8 Hz, 1H), 7.17 (d, J=8.7 Hz, 1H), 4.38 (s, 2H). HRMS (EI⁺) m/z: calc. 575.0323, found 575.0328.

### Example 2gg

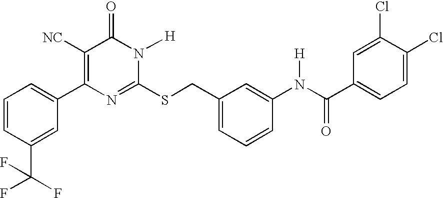

### 3,4-Dichloro-N-{3-[5-cyano-6-oxo-4-(3-trifluoromethyl-phenyl)-1,6-dihydro-pyrimidin-2-ylsulfanylmethyl]-phenyl}-benzamide

Step 1:

### 2-Mercapto-6-oxo-4-(3-trifluoromethyl-phenyl)-1,6-dihydro-pyrimidine-5-carbonitrile, piperidine Salt

Prepared following the procedure described in Example 1a, Step 1, Method A with the appropriate aldehyde. The crude reaction mixture was diluted with H₂O, saturated with NaCl, and extracted with EtOAc. The organic phase was washed with brine, dried over Na₂SO₄, filtered, concentrated in vacuo, and dried. The crude material was purified by repeated slurrying (EtOAc/hexanes) and filtering to provide 179 mg (8%) of semi-pure product as a light yellow solid. ¹H-NMR (DMSO-d₆) δ 11.73 (bs, 1H), 8.29 (bs, 2H), 8.03-8.08 (m, 2H), 7.86 (d, J=6.9 Hz, 1H), 7.73 (t, J=7.5 Hz, 1H), 3.01 (t, J=5.1 Hz, 4H), 1.54-1.64 (m, 6H).

Step 2:

Prepared following the procedure described in Example 2y, Step 3, using the piperidine salt product from Example 2gg, Step 1. The crude product was purified by silica gel chromatography (8% MeOH in dichloromethane) to provide 68 mg (57%) of the title compound as a white foam. ¹H-NMR (DMSO-d₆) δ 10.40 (s, 1H), 8.20 (d, J=1.8 Hz, 1H), 8.14-8.17 (m, 2H), 7.89-7.94 (m, 2H), 7.74-7.84 (m, 3H), 7.64 (d, J=9.0 Hz, 1H), 7.29 (t, J=8.4 Hz, 1H), 7.16 (d, J=8.4 Hz, 1H), 4.44 (s, 2H). HRMS (EI⁺) m/z: calc. 575.0323, found 575.0333.

### Example 2hh

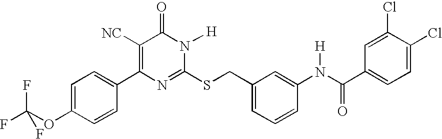

### 3,4-Dichloro-N-{3-[5-cyano-6-oxo-4-(4-trifluoromethoxy-phenyl)-1,6-dihydro-pyrimidin-2-ylsulfanylmethyl]-phenyl}-benzamide

Step 1:

### 2-Mercapto-6-oxo-4-(4-trifluoromethoxy-phenyl)-1,6-dihydro-pyrimidine-5-carbonitrile, piperidine Salt

Prepared following the procedure described in Example 1a, Step 1, Method A with the appropriate aldehyde. The crude reaction mixture was diluted with H₂O, saturated with NaCl, and extracted with EtOAc. The organic phase mixture was washed with brine, diluted with dichloromethane and MeOH, dried over Na₂SO₄, filtered, concentrated, and dried. The crude material was slurried in EtOAc, filtered, and washed with EtOAc to provide 472 mg (21%) of pure product as a fluffy, yellow solid. ¹H-NMR (DMSO-d₆) δ 11.68 (bs, 1H), 8.63 (bs, 2H), 7.86 (d, J=9.0 Hz, 2H), 7.46 (d, J=9.0 Hz, 2H), 2.98 (t, J=5.1 Hz, 4H), 1.55-1.66 (m, 6H).

Step 2:

Prepared following the procedure described in Example 2y, Step 3, using the piperidine salt product from Example 2hh, Step 1. The crude material was purified by silica gel chromatography (8% MeOH in dichloromethane) to provide 76 mg (51%) of the title compound as a white solid. ¹H-NMR (DMSO-d₆) δ 10.40 (s, 1H), 8.20 (d, J=2.1 Hz, 1H), 8.00 (d, J=8.2 Hz, 2H), 7.92 (dd, J=9.0, 2.7 Hz, 1H), 7.81-7.86 (m, 2H), 7.61 (d, J=8.4 Hz, 1H), 7.51 (d, J=8.2 Hz, 2H), 7.32 (t, J=8.4 Hz, 1H), 7.18 (d, J=7.2 Hz, 1H), 4.47 (s, 2H). HRMS (EI⁺) m/z: calc. 591.0272, found 591.0284.

### Example 2ii

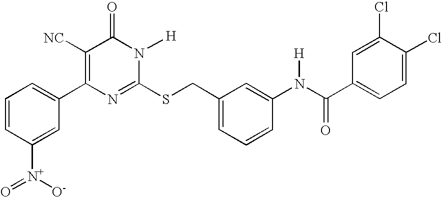

### 3,4-Dichloro-N-{3-[5-cyano-4-(3-nitro-phenyl)-6-oxo-1,6-dihydro-pyrimidin-2-ylsulfanylmethyl]-phenyl}-benzamide

Step 1:

### 2-Mercapto-4-(3-nitro-phenyl)-6-oxo-1,6-dihydro-pyrimidine-5-carbonitrile, piperidine Salt

Prepared following the procedure described in Example 1a, Step 1, Method A with the appropriate aldehyde and EtOH as solvent. The crude reaction mixture was filtered instead, and washed with EtOH several times and dried in vacuo to provide 3.49 g (37%) of pure product as a fluffy, yellow solid. ¹H-NMR (DMSO-d₆) δ 11.75 (bs, 1H), 8.50 (bs, 1H), 8.31 (dd, J=7.5, 0.6 Hz, 1H), 8.20 (m, 2H), 7.75 (t, J=8.1 Hz, 1H), 2.97 (t, J=5.7 Hz, 4H), 1.50-1.63 (m, 6H).

Step 2:

Prepared following the procedure described in Example 2y, Step 3, using the piperidine salt product from Example 2ii, Step 1 and using EtOH as solvent instead of DMF. The crude mixture was filtered and washed several times with EtOH and dried in vacuo to provide 118 mg (77%) of the title compound as a white solid. ¹H-NMR (DMSO-d₆) δ 10.32 (s, 1H), 8.69(t, J=2.4 Hz, 1H), 9.31-8.39 (m, 2H), 8.12 (d, J=2.7 Hz, 1H), 7.76-7.87 (m, 4H), 7.54 (d, J=9.6 Hz, 1H), 7.28 (t, J=8.4 Hz, 1H), 7.15 (d, J=7.8 Hz, 1H), 4.52 (s, 2H).

### Example 2ii

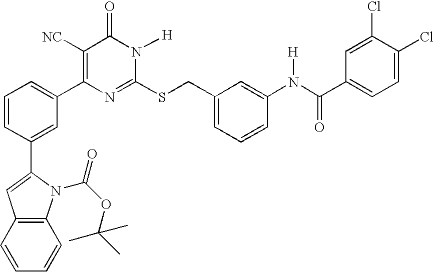

### 2-(3-{5-Cyano-2-[3-(3,4-dichloro-benzoylamino)-benzylsulfanyl]-6-oxo-1,6-dihydro-pyrimidin-4-yl}-phenyl)-indole-1-carboxylic acid tert-butyl ester

Step 1:

### 2-(3-Formyl-phenyl)-indole-1-carboxylic acid tert-butyl ester

Prepared following the procedure described in Example 1c Step 1 with the appropriate aldehyde and boronic acid. The crude material was purified by silica gel chromatography (10% EtOAc in hexanes) to provide 3.07 g (89%) of pure product as a yellow solid. ¹H-NMR (CDCl₃) δ 10.08 (s, 1H), 8.22 (d, J=7.8 Hz, 1H), 7.96 (s, 1H), 7.88 (d, J=6.9 Hz, 1H), 7.70 (d, J=6.9 Hz, 1H), 7.57-7.69 (m, 2H), 7.36 (t, J=7.5 Hz, 1H), 7.27 (t, J=7.2 Hz, 1H), 6.63 (s, 1H), 1.33 (s, 9H).

Step 2:

### 2-[3-(5-Cyano-2-mercapto-6-oxo-1,6-dihydro-pyrimidin-4-yl)-phenyl]-indole-1-carboxylic acid tert-butyl ester, piperidine Salt

Prepared as described in Example 1a, Step 1, Method A using the aldehyde obtained from Example 2jj, Step 1. Filtration of the crude reaction mixture followed by washing with EtOH provided 1.93 g (38%) of pure product as a pale yellow powder. ¹H-NMR (DMSO-d₆) δ 11.62 (bs, 1H), 8.18 (bs, 2H), 8.12 (d, J=9.0 Hz, 1H), 7.78-7.82 (m, 2H), 7.64 (d, J=7.2 Hz, 1H), 7.52-7.60 (m, 2H), 7.33-7.38 (m, 1H), 7.27 (t, J=7.2 Hz, 1H), 6.79 (s, 1H), 3.00 (t, J=5.1 Hz, 4H), 1.54-1.67 (m, 6H), 1.26 (s, 9H).

Step 3:

Prepared following the procedure described in Example 2y, Step 3, using the piperidine salt product from Example 2jj, Step 2. The crude material was purified by silica gel chromatography (5% MeOH in dichloromethane) to provide 677 mg (99%) of the title compound. ¹H-NMR (DMSO-d₆) δ 10.36 (s, 1H), 8.15 (d, J=0.9 Hz, 1H), 8.11 (d, J=7.8 Hz, 1H), 7.86-7.95 (m, 4H), 7.78 (d, J=8.4 Hz, 1H), 7.56-7.67 (m, 4H), 7.33-7.38 (m, 1H), 7.24-7.29 (m, 1H), 7.14-7.16 (m, 2H), 6.74 (s, 1H), 4.51 (s, 2H), 1.23 (s, 9H). HRMS (EI⁺) m/z: calc. 722.1395, found 722.1379.

### Example 2kk

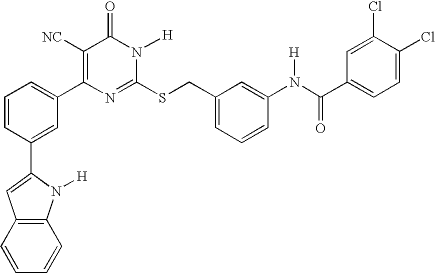

### 3,4-Dichloro-N-(3-{5-cyano-4-[3-(1H-indol-2-yl)-phenyl]-6-oxo-1,6-dihydro-pyrimidin-2-ylsulfanylmethyl}-phenyl)-benzamide

To a round-bottom flask was charged 2-(3-{5-cyano-2-[3-(3,4-dichloro-benzoylamino)-benzylsulfanyl]-6-oxo-1,6-dihydro-pyrimidin-4-yl}-phenyl)-indole-1-carboxylic acid tert-butyl ester obtained from Example 2jj, Step 3 (0.49 g, 0.68 mmol) and dichloromethane (20 mL). The solution was cooled in an ice bath and trifluoroacetic acid (6 mL) was added. HPLC analysis indicated the reaction was complete after 1 h. The solution was concentrated in vacuo, the residue was re-dissolved in toluene (30 mL), and the solution concentrated again. The crude material was purified by silica gel chromatography (3% MeOH in dichloromethane) to provide semi-pure product, which was further purified by slurrying in dichloromethane (25 mL) and MeOH (2 mL) for 1 h, follow by filtration, to provide 0.11 g (26%) of pure product as a light yellow powder. ¹H-NMR (DMSO-d₆) δ 11.61 (s, 1H), 10.41 (s, 1H), 8.20-8.22 (m, 2H), 7.94 (dd, J=8.4, 2.1 Hz, 2H), 7.80 (d, J=7.8 Hz, 1H), 7.77 (s, 1H), 7.64-7.71 (m, 2H), 7.51-7.56 (m, 2H), 7.40 (d, J=8.1 Hz, 1H), 7.29 (t, J=7.5 Hz, 1H), 7.18 (d, J=7.2 Hz, 1H), 7.10 (t, J=7.5 Hz, 1H), 6.99 (t, J=7.2 Hz, 1H), 6.90 (s, 1H), 4.34 (s, 2H). HRMS (EI⁺) m/z: calc. 622.0871, found 622.0861.

### Example 2ll

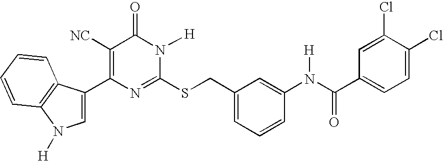

### 3,4-Dichloro-N-{3-[5-cyano-4-(1H-indol-3-yl)-6-oxo-1,6-dihydro-pyrimidin-2-ylsulfanylmethyl]-phenyl}-benzamide

Step 1:

6-(1H-Indol-3-yl)-4-oxo-2-thioxo-1,2,3,4-tetrahydro-pyrimidine-5-carbonitrile.

To a round-bottom flask was charged indole-3-carboxaldehyde (1.71 g, 11.8 mmol), thiourea (0.90 g, 11.8 mmol), ethyl cyanoacetate (1.26 mL, 11.8 mmol), EtOH (24 mL), and K₂CO₃ (1.65 g, 11.9 mmol) following the procedure of Kambe, S., et al, [Synthesis, 1979, pp 287-289]. The bright yellow mixture was heated at reflux for 10 h. HPLC indicated some intermediate remained. The mixture was filtered and the yellow-orange solid was washed with EtOH and THF. The solid was mixed with H₂O and heated for 10 min. on an 80° C. oil bath resulting in a clear, yellow solution. After cooling to room temperature, the solution was acidified with AcOH, resulting in a yellow precipitate. The mixture was filtered and the solids washed with H₂O. Drying provided ˜414 mg (13%) of pure product as a yellow solid. ¹H-NMR (DMSO-d₆) δ 12.99 (bs, 2H), 12.23 (bs, 1H), 8.11 (d, J=2.4 Hz, 1H), 7.63 (d, J=7.8 Hz, 1H), 7.53 (dd, J=7.8, 2.4 Hz, 1H), 7.19-7.29 (m, 2H).

Step 2:

To a round-bottom flask was charged 6-(1H-indol-3-yl)-4-oxo-2-thioxo-1,2,3,4-tetrahydro-pyrimidine-5-carbonitrile obtained from Example 2ll, Step 1 (˜414 mg, ˜1.54 mmol), N-(3-bromomethyl-phenyl)-3,4-dichloro-benzamide obtained from Example 2y, Step 2 (522 mg, 1.45 mmol), EtOH (21 mL), and diisopropylethylamine (0.25 mL, 1.44 mmol). The mixture was heated at reflux for 3 h. HPLC analysis indicated the reaction was complete. The mixture was filtered and washed with EtOH to provide 581 mg (77%) of the title compound as a yellow solid. ¹H-NMR (DMSO-d₆) δ 13.48 (bs, 1H), 12.17 (s, 1H), 10.43 (s, 1H), 8.49 (d, J=2.7 Hz, 1H), 8.17-8.20 (m, 2H), 7.91 (dd, J=9.3, 2.1 Hz, 1H), 7.89 (s, 1H), 7.82 (d, J=8.7 Hz, 1H), 7.68 (d, J=7.0 Hz, 1H), 7.51 (d, J=8.4 Hz, 1H), 7.33 (t, J=8.4 Hz, 1H), 7.18-7.23 (m, 2H), 7.09 (t, J=7.0 Hz, 1H), 4.70 (s, 2H).

### Example 2mm

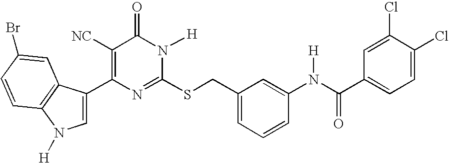

### N-{3-[4-(5-Bromo-1H-indol-3-yl)-5-cyano-6-oxo-1,6-dihydro-pyrimidin-2-ylsulfanylmethyl]-phenyl}-3,4-dichloro-benzamide

Step 1:

### 6-(5-Bromo-1H-indol-3-yl)-4-oxo-2-thioxo-1,2,3,4-tetrahydro-pyrimidine-5-carbonitrile

Prepared as in Example 2ll, Step 1, using the appropriate aldehyde. The isolated material contained an intermediate and was resubjected to reflux conditions for 6.5 h in the presence of thiourea (0.86 g), EtOH (40 mL), and K₂CO₃ (1.57 g). Repeated workup provided 0.57 g (10%) of pure product as a light yellow solid. ¹H-NMR (DMSO-d₆) δ 13.00 (bs, 2H), 12.34 (s, 1H), 8.14 (d, J=2.3 Hz, 1H), 7.83 (s, 1H), 7.50 (d, J=8.7 Hz, 1H), 7.38 (dd, J=8.7, 2.3 Hz, 1H).

Step 2:

Prepared following the procedure outlined in Example 2ll, Step 2 using the product obtained from Example 2mm, Step 1. The reaction mixture was filtered and the solids washed with EtOH to provide 0.98 g (95%) of the title compound as a yellow solid. ¹H-NMR (DMSO-d₆) δ 13.55 (bs, 1H), 12.34 (s, 1H), 10.43 (s, 1H), 8.55 (d, J=2.7 Hz, 2H), 8.19 (d, J=2.1 Hz, 1H), 7.89-7.93 (m, 2H), 7.81 (d, J=8.1 Hz, 1H), 7.68 (d, J=7.8 Hz, 1H), 7.51 (d, J=8.1 Hz, 1H), 7.30-7.37 (m, 2H), 7.22 (d, J=7.2 Hz, 1H), 4.70 (s, 2H). HRMS (EI⁺) m/z: calc. 623.9485, found 623.9490.

### Example 2nn

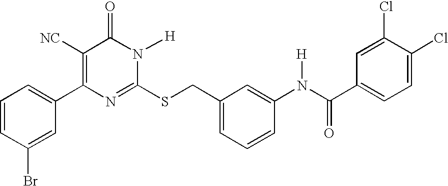

### N-{3-[4-(3-Bromo-phenyl)-5-cyano-6-oxo-1,6-dihydro-pyrimidin-2-ylsulfanylmethyl]-phenyl}-3,4-dichloro-benzamide

Step 1:

### 4-(3-Bromo-phenyl)-2-mercapto-6-oxo-1,6-dihydro-pyrimidine-5-carbonitrile, piperidine Salt

Prepared following the procedure described in Example 1a, Step 1, Method A using the appropriate aldehyde. The crude reaction mixture was concentrated, diluted with H₂O, saturated with NaCl, and extracted with EtOAc. The organic phase mixture was filtered and washed with EtOAc to provide 2.71 g (32%) of pure product as a yellow solid. ¹H-NMR (DMSO-d₆) δ 11.60 (bs, 1H), 8.26 (bs, 2H), 7.87 (s, 1H), 7.75-7.78 (m, 1H), 7.68-7.71 (m, 1H), 7.44 (t, J=8.4 Hz, 1H), 3.99 (t, J=5.1 Hz, 4H), 1.54-1.66 (m, 6H).

Step 2:

Prepared following Example 2y, Step 3 with EtOH as solvent and using the product obtained from Example 2nn, Step 1 as the piperidine salt. The reaction mixture was diluted with EtOH (5 mL), stirred rapidly, and filtered. The solids were washed with EtOH to provide 1.33 g (89%) of pure product as a beige solid. ¹H-NMR (DMSO-d6) δ 10.40 (s, 1H), 8.19 (d, J=2.1 Hz, 1H), 8.03 (s, 1H), 7.89-7.94 (m, 3H), 7.79-7.85 (m, 2H), 7.62 (d, J=8.1 Hz, 1H), 7.53 (t, J=7.8 Hz, 1H), 7.33 (t, J=8.4 Hz, 1H), 7.19 (d, J=7.5 Hz, 1H), 4.55 (s, 2H). HRMS (EI⁺) m/z: calc. 584.9554, found 584.9551.

### Example 2oo

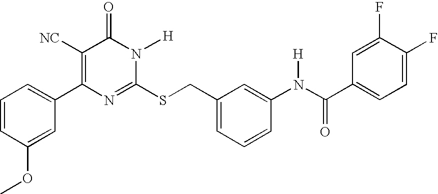

### N-{3-[5-Cyano-4-(3-methoxy-phenyl)-6-oxo-1,6-dihydro-pyrimidin-2-ylsulfanylmethyl]-phenyl}-3,4-difluoro-benzamide

Step 1:

### 3,4-Difluoro-N-(3-hydroxymethyl-phenyl)-benzamide

To a round-bottom flask was charged 3-aminobenzyl alcohol (4.93 g, 40.0 mmol), dioxane (40 mL), and triethylamine (6.1 mL, 44 mmol). As solution of 3,4-difluorobenzoylchloride (5.0 mL, 40 mmol) in dioxane (40 mL) was added via addition funnel over 30 min. After stirring overnight, HPLC analysis indicated the reaction was complete. The beige mixture was diluted with H₂O (300 mL) and acidified to pH 1 with 1N HCl (˜15 mL). The mixture was stirred rapidly, filtered, and the solids washed with H₂O. The solids were dried in a vacuum oven (40° C.) overnight to provide 9.27 g (88%) of pure product as a fluffy, beige solid. ¹H-NMR (DMSO-d₆) δ 10.29 (s, 1H), 8.01-8.08 (m, 1H), 7.86-7.90 (m, 1H), 7.74 (s, 1H), 7.58-7.67 (m, 2H), 7.31 (t, J=7.4 Hz, 1H), 7.06 (d, J=7.9 Hz, 1H), 5.23 (t, J=5.2 Hz, 1H), 4.51 (d, J=5.2 Hz, 2H).

Step 2:

### N-(3-Bromomethyl-phenyl)-3,4-difluoro-benzamide

Prepared following the procedure in Example 2y, Step 2 using the product from Example 2oo, Step 1. The solids were dried in a vacuum oven (40° C.) overnight to provide 4.74 g (96%) of pure product as a beige solid. ¹H-NMR (DMSO-d₆) δ 10.38 (s, 1H), 8.01-8.08 (m, 1H), 7.85-7.89 (m, 2H), 7.59-7.70 (m, 2H), 7.36 (t, J=7.2 Hz, 1H), 7.20 (d, J=7.2 Hz, 1H), 4.72 (s, 2H).

Step 3:

To a round-bottom flask was charged mercapto-4-(3-methoxy-phenyl)-6-oxo-1,6-dihydro-pyrimidine-5-carbonitrile, piperidine salt, from Example 1a, Step 1, Method A (102 mg), N-(3-bromomethyl-phenyl)-3,4-difluoro-benzamide, from Example 2oo, Step 2 (100 mg), and EtOH (6 mL). The solids dissolved as the reaction was heated at reflux for 6 h; HPLC analysis indicated the reaction was complete. The reaction was cooled to room temperature and a precipitate formed. The mixture was filtered and the solids washed with EtOH to provide 100 mg (67%) of the title compound as a white solid. ¹H-NMR (DMSO-d₆) δ 10.32 (s, 1H), 7.98-8.05 (m, 1H), 7.83-7.87 (m, 2H), 7.58-7.67 (m, 2H), 7.44-7.53 (m, 3H), 7.31 (t, J=8.7 Hz, 1H), 7.15-7.20 (m, 2H), 4.56 (s, 2H), 3.78 (s, 3H). HRMS (EI⁺) m/z: calc. 505.1146, found 505.1131.

### Examples 2pp through 2ooo

Examples 2pp-2ooo were prepared from the p-amino intermediate 2-(4-amino-benzylsulfanyl)-4-(3-methoxy-phenyl)-6-oxo-1,6-dihydro-pyrimidine-5-carbonitrile whose synthesis is described below.

Step 1:

### (4-Hydroxymethyl-phenyl)-carbamic acid tert-butyl ester

To a 250 mL round bottom flask was added (4-amino-phenyl)-methanol (4.13 g, 33.6 mmol), dichloromethane (50 mL) followed by di-tert-butyl dicarbonate (8.5 g, 36.9 mmol). The mixture was allowed to stir for 18 h under a nitrogen atmosphere. By TLC, a small amount of starting amine remained, which reacted during concentration by rotary evaporation. The product was purified by column chromatography (ethyl acetate:hexanes, 1:1) to yield (4-hydroxymethyl-phenyl)-carbamic acid tert-butyl ester (7.36 g, 33.0 mmol, 98%) as a white solid.

Step 2:

### (4-Bromomethyl-phenyl)-carbamic acid tert-butyl ester

To a 1 L round bottom flask was added (4-hydroxymethyl-phenyl)-carbamic acid tert-butyl ester (22.3 g, 0.10 mol) obtained from Examples 2pp through 2ooo, Step 1, dichloromethane (400 mL) and triphenylphospine (31.5 g, 0.12 mol). Upon cooling to 0° C., N-bromosuccinimide (19.6 g, 0.11 mmol) was added in small portions and the reaction was allowed to warm to room temperature and stirred under nitrogen for 1 h. The mixture was transferred into a large flask and hexanes were added until the solution became turbid. The solution was eluted through a plug of silica (300 g) with 1:9 ethyl acetate:hexanes and concentrated to yield (4-bromomethyl-phenyl)-carbamic acid tert-butyl ester (12.8 g, 0.057 mol, 57%) as a white solid which was sufficiently pure by ¹H NMR to proceed to the next step: ¹H NMR (300 MHz, CDCl₃) δ 8.00 (s, 1H), 7.52 (d, 2H), 7.04 (d, 2H), 4.56 (s, 2H), 1.40 (s, 9H).

Step 3:

### {4-[5-Cyano-4-(3-methoxy-phenyl)-6-oxo-1,6-dihydro-pyrimidin-2-ylsulfanylmethyl}-phenyl}-carbamic acid tert-butyl ester

To a 250 mL round bottom flask was added mercapto-4-(3-methoxy-phenyl)-6-oxo-1,6-dihydro-pyrimidine-5-carbonitrile, piperidine salt (3.92 g, 11.4 mmol) obtained from Example 1a, Step 1, Method A, DMF (50 mL), followed by (4-bromomethyl-phenyl)-carbamic acid tert-butyl ester (3.42, 12.0 mmol) obtained from Examples 2pp through 2ooo, Step 2. After stirring for 18 h, the mixture was added to water (300 mL) and extracted with ethyl acetate (3×150 mL). The combined organic phase was washed with brine (300 mL), dried (MgSO₄) and concentrated by rotary evaporation. The residue was triturated with ethyl ether and hexanes to yield pure product (4.80 g, 10.3 mmol, 90%) as a white solid. ¹H NMR (300 MHz, DMSO-d₆) δ 9.35 (s, 1H), 7.53-7.48 (m, 3H), 7.37 (d, 2H, J=8.58 Hz), 7.29 (d, 2H, J=8.58 Hz), 7.21-7.19 (m, 1H), 4.47 (s, 2H), 3.81 (s, 3H), 1.45 (s, 9H); APCI MS (negative mode) m/z 463 [M−H]⁻.
Step 4:

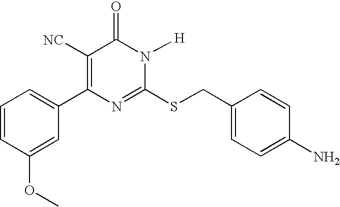

### 2-(4-Amino-benzylsulfanyl)-4-(3-methoxy-phenyl)-6-oxo-1,6-dihydro-pyrimidine-5-carbonitrile

To a 1 L round bottom flask was added {4-[5-Cyano-4-(3-methoxy-phenyl)-6-oxo-1,6-dihydro-pyrimidin-2-ylsulfanylmethyl]-phenyl}-carbamic acid tert-butyl ester (3.74 g, 10.3 mmol) obtained from Examples 2pp through 2ooo, Step 3, phenol (9.7 g, 103.0 mmol) and dichloromethane (300 mL). Under a nitrogen atmosphere, tetrachlorosilane (5.9 mL, 51.5 mmol) was added dropwise and the mixture was allowed to stir for 4 h. The solvent was evaporated under vacuum and the pale yellow solid was triturated with several volumes of acetonitrile to yield the free amine (2.34 g, 6.42 mmol, 62%) as an off-white solid: ¹H NMR (300 MHz, DMSO-d₆) δ 11.00-10.00 (br, 2H), 7.11-7.59 (m, 9H), 4.56 (s, 2H), 3.79 (s, 3H); APCI MS (negative mode) m/z 363 [−H]⁻.

The amide formation step for Examples 2pp-2ooo followed the procedure outlined in Example 2a using the amine prepared in Step 4 above [2-(4-amino-benzylsulfanyl)-4-(3-methoxy-phenyl)-6-oxo-1,6-dihydro-pyrimidine-5-carbonitrile] and the appropriate functionalized carbonyl derivative.

### Example 3

### Preparation of Examples 3a through 3bb

| | | Mass Spec |
|---|---|---|
| Example | structure | Data |
| 3a | 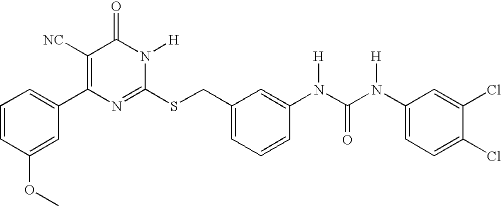 | HRMS (EI⁺) m/z: calc. 552.0664, found 552.0677 |
| 3b | 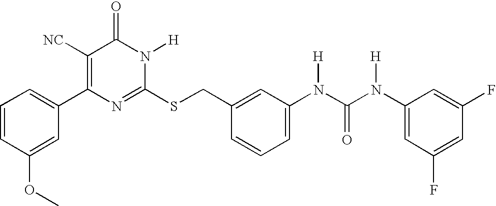 | APCI MS m/z 520.4 [M + H]⁺ |
| 3c | 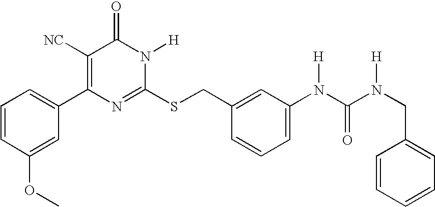 | APCI MS m/z 498.2 [M + H]⁺ |
| 3d | 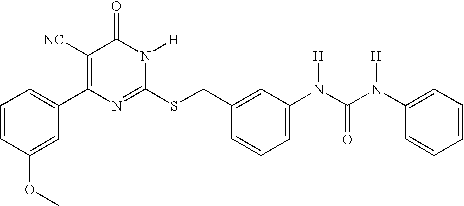 | APCI MS m/z 484.2 [M + H]⁺ |
| 3e | 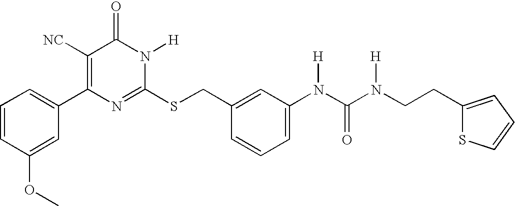 | APCI MS m/z 518.4 [M + H]⁺ |
| 3f | 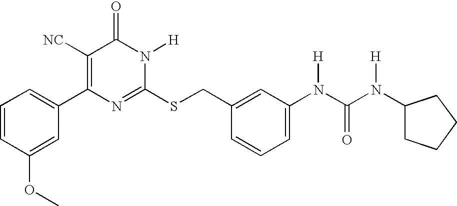 | APCI MS m/z 476.2 [M + H]⁺ |
| 3g | 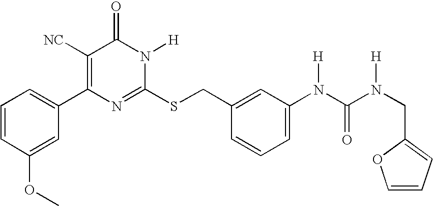 | APCI MS m/z 488.2 [M + H]⁺ |
| 3h | 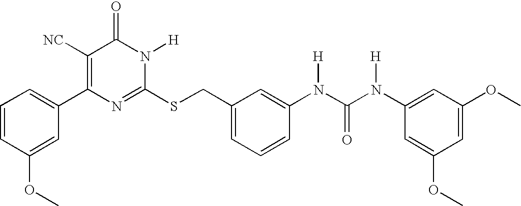 | APCI MS m/z 544.2 [M + H]⁺ |
| 3i | 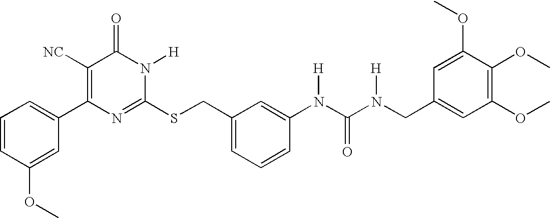 | APCI MS m/z 588.2 [M + H]⁺ |
| 3j | 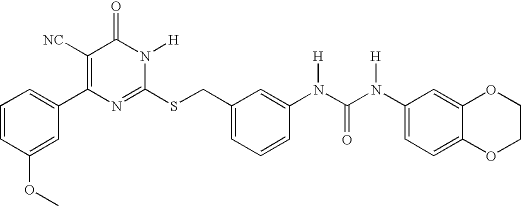 | APCI MS m/z 542.4 [M + H]⁺ |
| 3k | 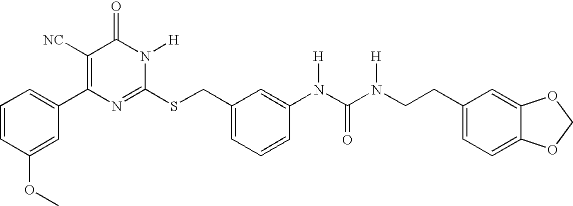 | APCI MS m/z 556.0 [M + H]⁺ |
| 3l | 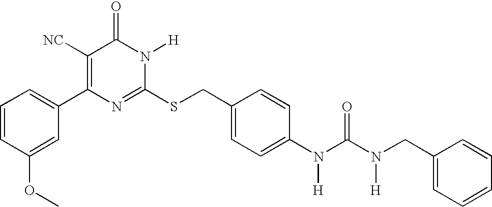 | APCI MS m/z 496.2 [M − H]⁻ |
| 3m | 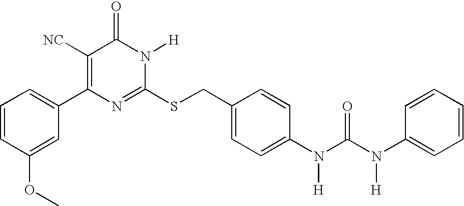 | APCI MS m/z 482.0 [M − H]⁻ |
| 3n | 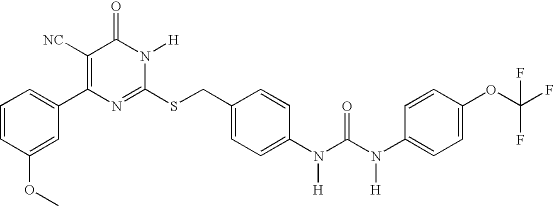 | APCI MS m/z 656.6 [M − H]⁻ |
| 3o | 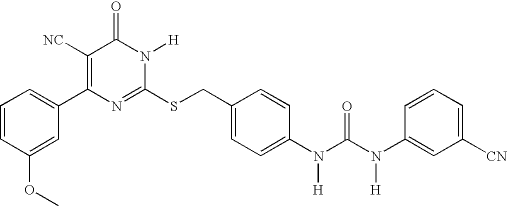 | APCI MS m/z 507.2 [M − H]⁻ |
| 3p | 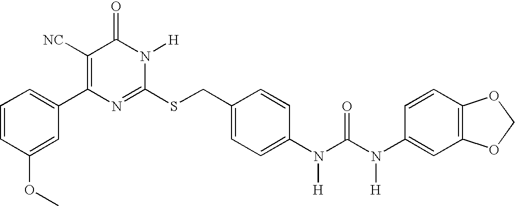 | APCI MS m/z 526.2 [M − H]⁻ |
| 3q | 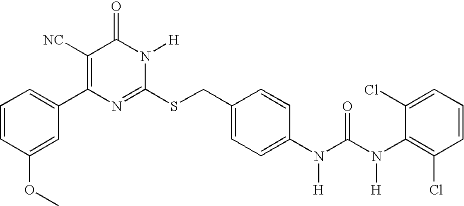 | APCI MS m/z 550.2 [M − H]⁻ |
| 3r | 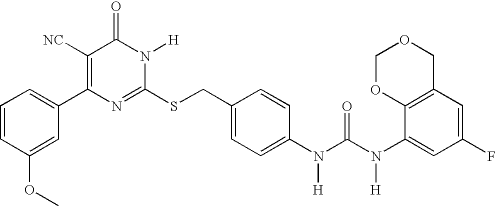 | APCI MS m/z 558.2 [M − H]⁻ |
| 3s | 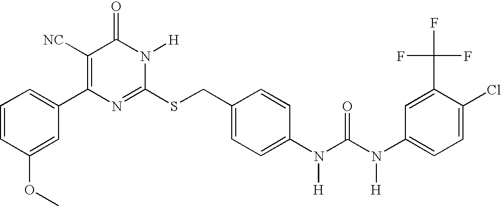 | APCI MS m/z 584.0 [M − H]⁻ |
| 3t | 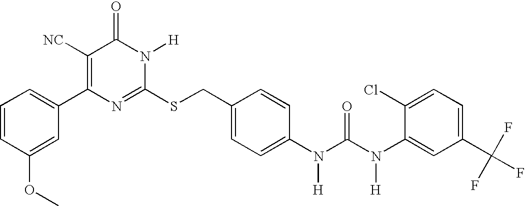 | APCI MS m/z 584.0 [M − H]⁻ |
| 3u | 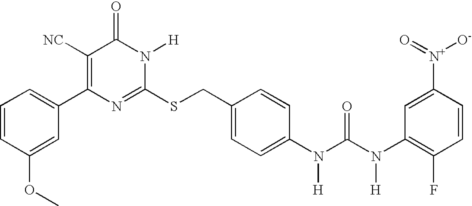 | APCI MS m/z 545.7 [M − H]⁻ |
| 3v | 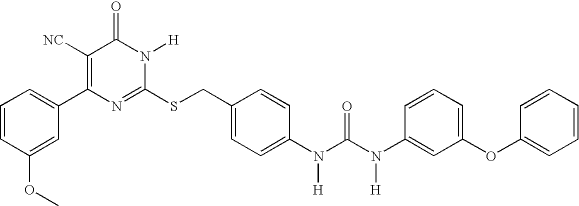 | APCI MS m/z 574.2 [M − H]⁻ |
| 3w | 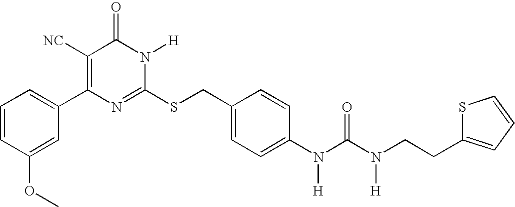 | APCI MS m/z 516.2 [M − H]⁻ |
| 3x | 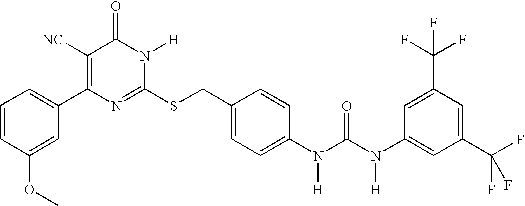 | APCI MS m/z 618.2 [M − H]⁻ |
| 3y | 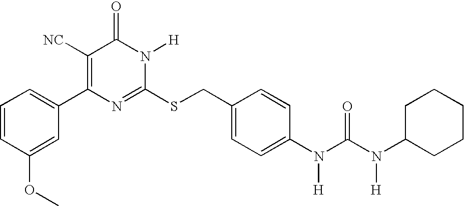 | APCI MS m/z 488.4 [M − H]⁻ |
| 3z | 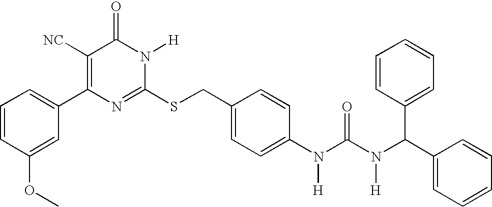 | APCI MS m/z 572.2 [M − H]⁻ |
| 3aa | 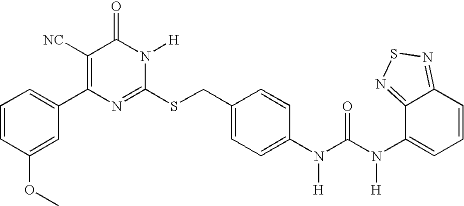 | APCI MS m/z 540.4 [M − H]⁻ |
| 3bb | 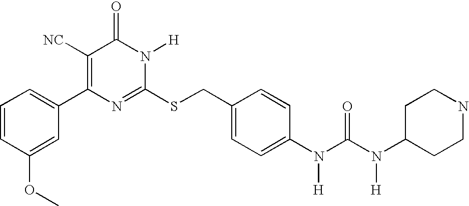 | APCI MS m/z 489.4 [M − H]⁻ |

### Example 3a

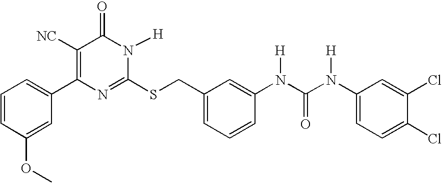

### 1-{3-[5-Cyano-4-(3-methoxy-phenyl)-6-oxo-1,6-dihydro-pyrimidin-2-ylsulfanylmethyl]-phenyl}-3-(3,4-dichloro-phenyl)-urea

To a round-bottom flask was charged 2-(3-amino-benzylsulfanyl)-4-(3-methoxy-phenyl)-6-oxo-1,6-dihydro-pyrimidine-5-carbonitrile prepared from Example 1a, Step 3 (104 mg, 0.285 mmol), 3,4-dichlorophenylisocyanate (56 mg, 0.298 mmol), and THF (4 mL). After stirring for 2 h 15 min., HPLC analysis indicated the reaction was complete. The solution was diluted with brine and H₂O and extracted with EtOAc. The organics were washed with brine, dried over Na₂SO₄, filtered, concentrated, and dried. The crude material was purified by silica gel chromatography (7.5% MeOH in dichloromethane) to provide 94 mg (60%) of the title compound as an off-white solid. ¹H-NMR (DMSO-d₆) δ 9.00 (s, 1H), 8.45 (s, 1H), 7.80 (d, J=2.7 Hz, 1H), 7.58 (s, 1H), 7.44-7.53 (m, 4H), 7.33 (d, J=2.1 Hz, 1H), 7.30 (d, J=2.7 Hz, 1H), 7.23 (t, J=7.5 Hz, 1H), 7.13-7.17 (m, 1H), 7.04 (d, J=6.9 Hz, 1H), 4.50 (s, 2H), 3.78 (s, 3H). HRMS (EI⁺) m/z: calc. 552.0664, found 552.0677.

### Example 3b

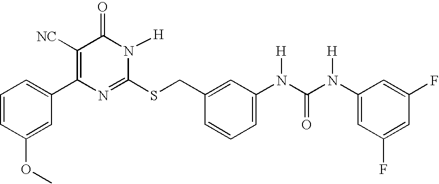

### 1-{3-[5-Cyano-4-(3-methoxy-phenyl)-6-oxo-1,6-dihydro-pyrimidin-2-ylsulfanylmethyl]-phenyl}-3-(3,5-difluoro-phenyl)-urea

To a round-bottom flask was charged 2-(3-amino-benzylsulfanyl)-4-(3-methoxy-phenyl)-6-oxo-1,6-dihydro-pyrimidine-5-carbonitrile prepared from Example 1a, Step 3 (210 mg, 0.58 mmol), polyvinylpyridine/10% (w/w) polymer-supported dimethylaminopyridine (0.16 g), and THF (5 mL). To the stirring mixture was added 3,5-difluorophenylisocyanate (215 mg, 1.38 mmol), and stirred at ambient temperature for 18 h. The resin was filtered, and the solvent concentrated in vacuo. The residue was purified by trituration with CH₃CN/MeOH (10:1) to yield 0.031 g, (10%) of the title compound as a white solid. ¹H-NMR (DMSO-d₆) δ 9.05 (s, 1H), 8.85 (s, 1H), 7.43-7.61 (m, 4H), 7.12-7.38 (m, 6H), 7.08 (d, 1H), 6.79 (m, 1H), 4.52 (s, 2H), 3.78 (s, 3H); APCI MS m/z 520 [M+H]⁺.

### Examples 3c-3bb

Examples 3c through 3bb were prepared following the procedure described in Example 3b with substitution of the appropriate isocyanate respectively.

### Example 4

### Preparation of Examples 4a through 4ff

| | | Mass Spec |
|---|---|---|
| Example | structure | Data |
| 4a | 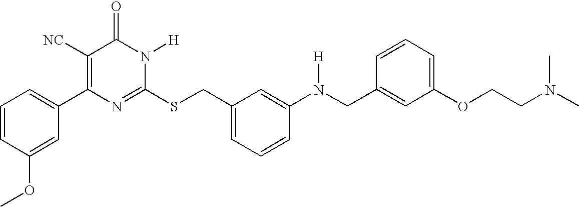 | HRMS (EI⁺) m/z: calc. 542.2226, found 542.2216 |
| 4b | 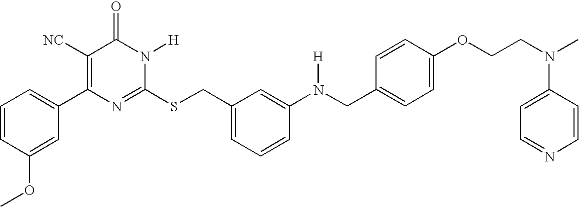 | HRMS (EI⁺) m/z: calc. 605.2335, found 605.2341 |
| 4c | 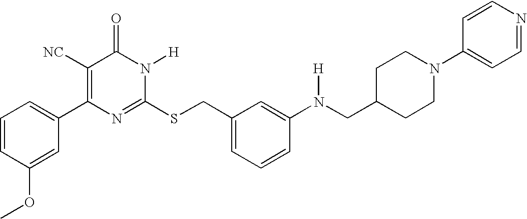 | HRMS (EI⁺) m/z: calc. 539.2229, found 539.2230 |
| 4d | 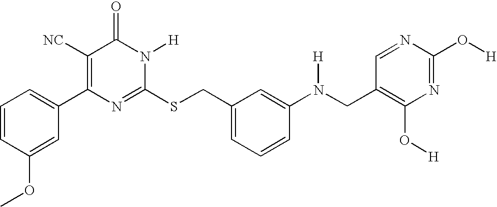 | HRMS (EI⁺) m/z: calc. 489.1345, found 489.1345 |
| 4e | 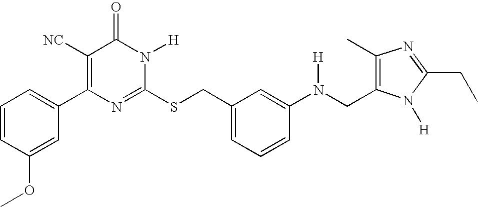 | HRMS (EI⁺) m/z: calc. 487.1916, found 487.1924 |
| 4f | 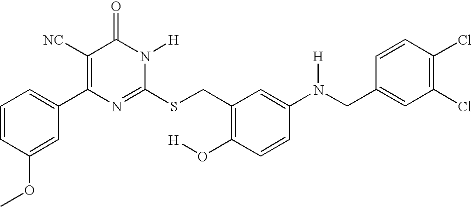 | HRMS (EI⁺) m/z: calc. 539.0711, found 539.0720 |
| 4g | 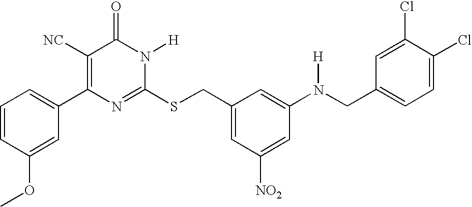 | HRMS (EI⁺) m/z: calc. 568.0613, found 568.0623 |
| 4h | 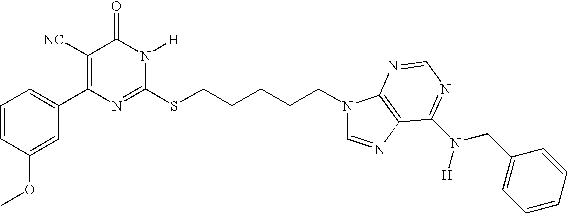 | HRMS (EI⁺) m/z: calc. 553.2134, found 553.2141 |
| 4i | 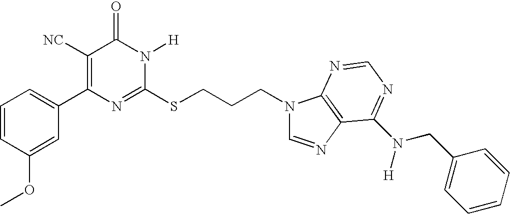 | HRMS (EI⁺) m/z: calc. 525.1821, found 525.1814 |
| 4j | 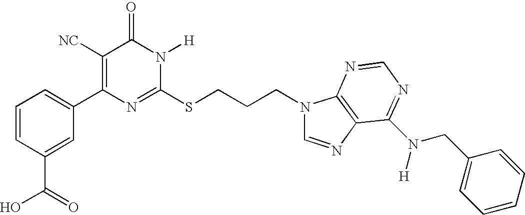 | HRMS (EI⁺) m/z: calc. 539.1614, found 539.1602 |
| 4k | 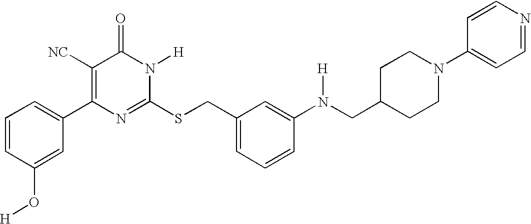 | HRMS (EI⁺) m/z: calc. 525.2072, found 525.2087 |
| 4l | 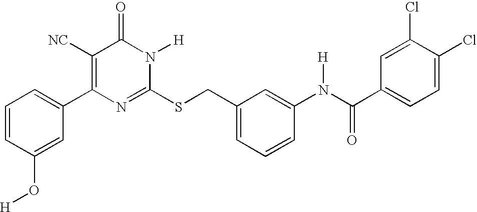 | HRMS (EI⁺) m/z: calc. 523.0398, found 523.0402 |
| 4m | 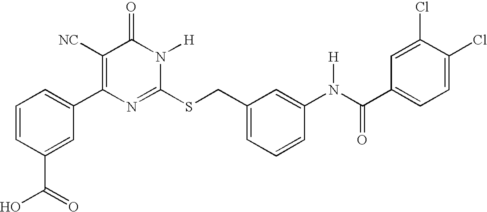 | TOF MS (ES⁺) m/z 551 [M + H]⁺ |
| 4n | 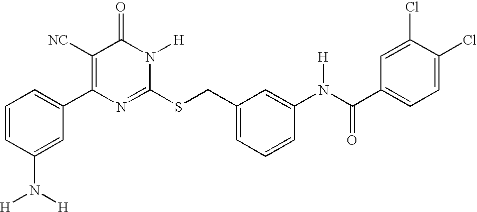 | TOF MS (ES⁺) m/z 522 [M + H]⁺ |
| 4o | 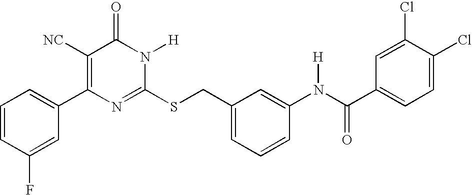 | HRMS (EI⁺) m/z: calc. 525.0355, found 525.0361 |
| 4p | 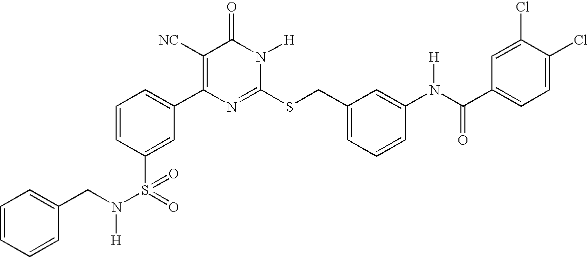 | HRMS (EI⁺) m/z: calc. 676.0647, found 676.0646 |
| 4q | 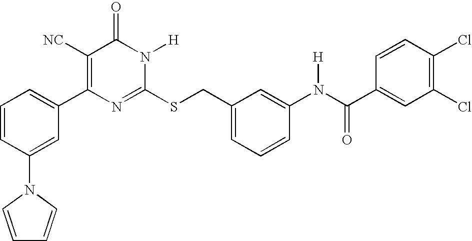 | HRMS (EI⁺) m/z: calc. 572.0715, found 572.0716 |
| 4r | 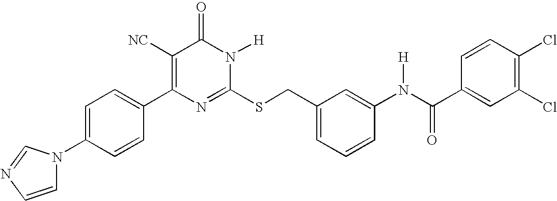 | HRMS (EI⁺) m/z: calc. 573.0667, found 573.0676 |
| 4s | 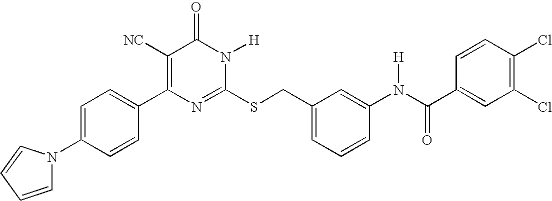 | HRMS (EI⁺) m/z: calc. 572.0715, found 572.0715 |
| 4t | 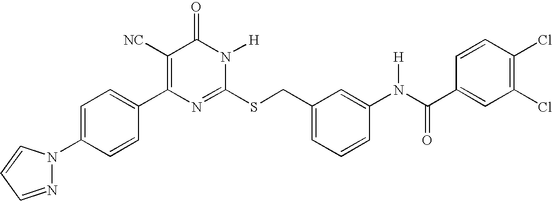 | HRMS (EI⁺) m/z: calc. 573.0667, found 573.0677 |
| 4u | 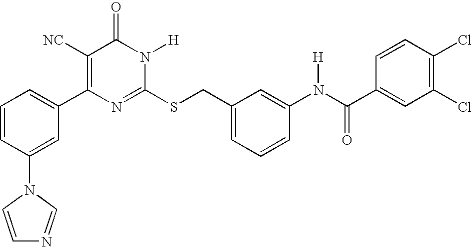 | HRMS (EI⁺) m/z: calc. 573.0667, found 573.0657 |
| 4v | 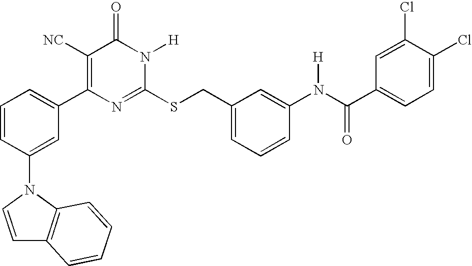 | HRMS (EI⁺) m/z: calc. 622.0871, found 622.0865 |
| 4w | 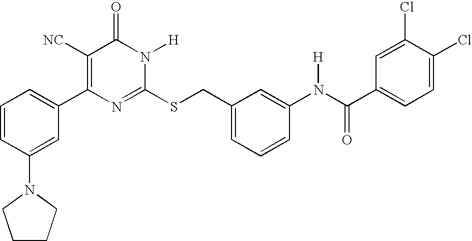 | HRMS (EI⁺) m/z: calc. 576.1028, found 576.1022 |
| 4x | 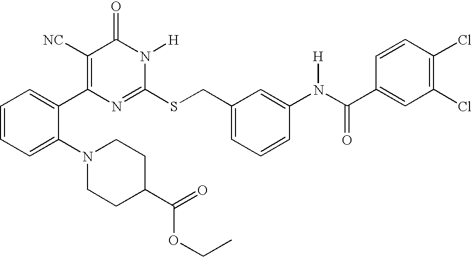 | HRMS (EI⁺) m/z: calc. 662.1395, found 662.1407 |
| 4y | 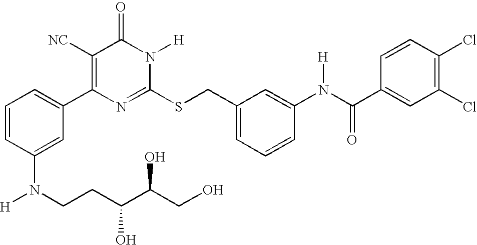 | HRMS (EI⁺) m/z: calc. 640.1188, found 640.1188 |
| 4z | 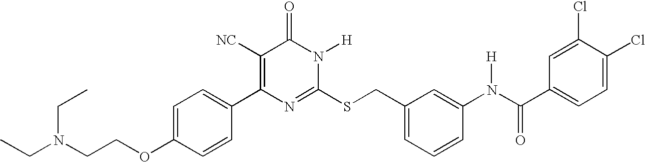 | HRMS (EI⁺) m/z: calc. 622.1446, found 622.1436 |
| 4aa | 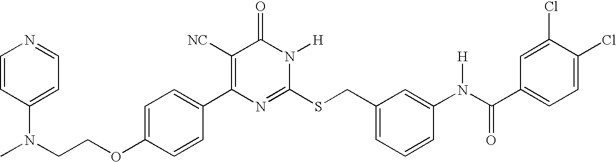 | HRMS (EI⁺) m/z: calc. 657.1242, found 657.1236 |
| 4bb | 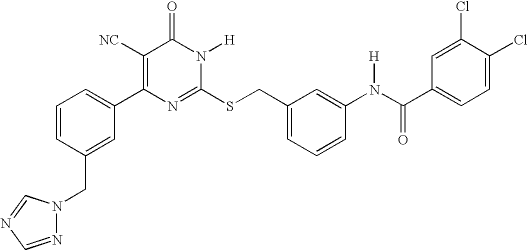 | HRMS (EI⁺) m/z: calc. 588.0776, found 588.0776 |
| 4cc | 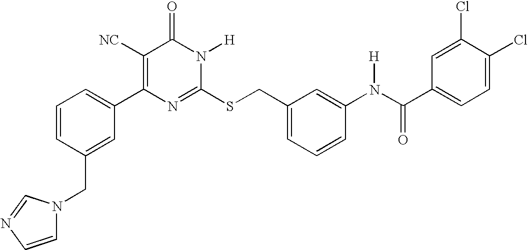 | HRMS (EI⁺) m/z: calc. 587.0824, found 587.0837 |
| 4dd | 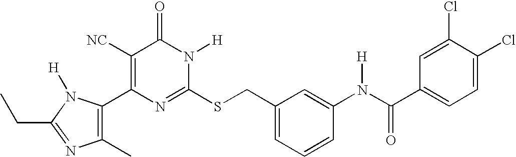 | HRMS (EI⁺) m/z: calc. 539.0824, found 539.0931 |
| 4ee | 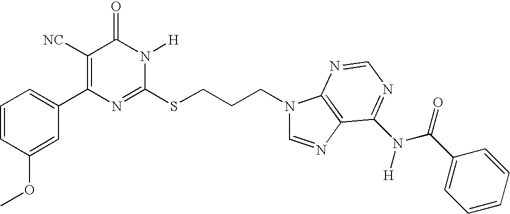 | HRMS (EI⁺) m/z: calc. 539.1614, found 539.1624 |
| 4ff | 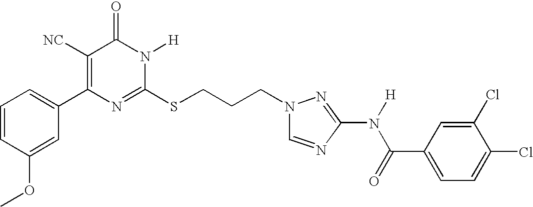 | HRMS (EI⁺) m/z: calc. 556.0725, found 556.0710 |

### Examples 4a-4ff

Examples 4a through 4ff were prepared following the procedures described herein and modifications thereof and by techniques from conventional organic chemistry repertoires as known to those skilled in the art.

### Example 5

### Time-Resolved Fluorescence Resonance Energy Transfer (TR-FRET) Assay

A TR-FRET kinase assay was used to screen potential inhibitors of MEKK2. The assay was performed as follows: Compounds to be tested were weighed and solubilized in DMSO (Sigma) to a stock concentration of 10 mM. Serial dilutions starting from 1 mM were prepared with additional DMSO. These dilutions were further diluted to 250 μM in kinase reaction buffer [20 mM Hepes pH 7.5, 5 mM MgCl₂, 1 mM DTT (Dithiothreitol-Sigma), 1 mM NaVO₄ (sodium vanadate-Sigma). A 25 μl aliquot of this solution was transferred to a black 96-well nonbinding surface microplate (Fisher). A stock aliquot containing 50-1125 ng of murine MEKK2 (mMEKK2) was diluted in the kinase reaction buffer and incubated with the compounds on the microplate for 10 minutes. A 25 μl aliquot of a 138 ng solution of biotin MBP (myelin basic protein—Upstate Biotechnology) was added to the reaction followed by 25 ul of a 25 ng solution of anti-phospho MBP antibody (Upstate Biotechnology) diluted in kinase reaction buffer. Finally an aliquot (50 μl) of a 5 uM solution of ATP (adenosine triphosphate) was added and the reaction was allowed to proceed for 60 minutes at room temperature. The reaction was stopped by the addition of 50 μL of a 12.5 ng solution of europium labeled anti-mouse IgG (Perkin Elmer) and 45 ng of Cy5 labeled streptavidin (Jackson Immunoresearch) in termination buffer (20 mM Hepes pH 7.5, 100 mM EDTA; Sigma). The assay was allowed to equilibrate for 45 minutes before placing the plate on the Victor™ plate reader. The plate was read using the 615/665 LANCE protocol. Results of the TR-FRET kinase assay are shown in Table 3.

### Example 6

### MEKK2 PhosphoELISA

A phospho-antibody enzyme-linked immunosorbent assay (PhosphoELISA) was used to screen potential inhibitors of MEKK2. The assay was performed as follows: Compounds to be tested were weighed and solubilized in DMSO (Sigma) to a stock concentration of 10 mM. Serial dilutions starting from 1 mM were prepared with additional DMSO. These dilutions were further diluted to 250 μM in kinase reaction buffer [20 mM Hepes pH 7.5, 5 mM MgCl₂, 1 mM DTT (Dithiothreitol-Sigma), 1 mM NaVO₄ (sodium vanadate- Sigma). A 25 μl aliquot of this solution was transferred to a glutathione coated 96-well microplate (Pierce). A stock aliquot containing 50-125 ng of murine MEKK2 (mMEKK2) was added to each well and incubated on the microplate for 10 minutes at room temperature. A 25 μl solution of MKK4/SKK1 (unactive) was diluted 1:200 in kinase assay buffer and added to the reaction plate followed by 50 μl of 25 μM ATP (adenosine triphosphate) to afford a final well concentration of 10 μM. The reaction was allowed to proceed for 60 minutes at room temperature before washing 3 times with wash buffer [100 mM phosphate pH 7.5, 0.05% TWEEN 20 (Sigma)]. A solution of anti-phospho MKK4/SEK1 antibody (Calbiochem) and biotin anti rabbit IgG (Jackson Immunoresearch) was diluted 1:2500 in kinase reaction buffer and added to the appropriate wells. The reaction was incubated for 60 minutes at room temperature. The microplate was washed 3 times with wash buffer and 100 ul of streptavidin horseradish peroxidase (HRP-Pierce) was added and incubated for 30 minutes at room temperature. The microplate was washed 3 times with wash buffer and 100 μl of TMB (3,3′,5,5′-tetramethylbenzidine-Sigma) was added. The assay was allowed to develop for 15-20 minutes then the reaction was stopped with the addition of 100 μl of 0.2 N sulfuric acid. The optical density (O.D.) of the plate was read on the Victor™ plate reader using the absorbance at 450 nm. Results of the PhosphoELISA assay are shown in Table 3.

TABLE 3
Biological results of TR-FRET kinase assay and MEKK2
PhosphoELISA assay.

| | mMEKK2/MBP | mMEKK2/MKK4 |
|---|---|---|
| Example | IC₅₀ (μM) | IC₅₀ (μM) |
| 1a | <1 | <1 |
| 1b | <5 | <1 |
| 1c | <5 | <1 |
| 1d | <5 | <5 |
| 1e | <20 | |
| 1f | <5 | |
| 1g | <40 | |
| 1h | <5 | |
| 1i | <10 | |
| 1j | <20 | |
| 1k | 40 | |
| 1l | <20 | |
| 1m | <20 | |
| 1n | <40 | |
| 1o | >40 | |
| 1p | 40 | |
| 1q | <1 | |
| 1r | <20 | |
| 1s | <10 | |
| 1t | <5 | |
| 1u | <10 | |
| 1v | <1 | <1 |
| 1w | <5 | |
| 1x | <1 | |
| 1y | <10 | |
| 1z | <40 | |
| 1aa | <1 | |
| 1bb | <1 | |
| 1cc | <1 | <1 |
| 1dd | <1 | |
| 2a | <5 | |
| 2b | <5 | |
| 2c | <5 | |
| 2d | <5 | |
| 2e | <5 | |
| 2f | <10 | |
| 2g | <5 | |
| 2h | <5 | <5 |
| 2i | <10 | <10 |
| 2j | <5 | |
| 2k | <10 | |
| 2l | <5 | |
| 2m | <5 | |
| 2n | 40 | |
| 2o | <5 | |
| 2p | <5 | |
| 2q | <5 | |
| 2r | <20 | |
| 2s | <10 | |
| 2t | <40 | |
| 2u | <20 | |
| 2v | <20 | |
| 2w | <40 | |
| 2x | <10 | |
| 2y | <5 | <1 |
| 2z | <1 | <1 |
| 2aa | | <1 |
| 2bb | <5 | <1 |
| 2cc | | <1 |
| 2dd | <5 | <5 |
| 2ee | <10 | <1 |
| 2ff | <5 | <5 |
| 2gg | >40 | <5 |
| 2hh | <40 | <20 |
| 2ii | <5 | <1 |
| 2jj | | <1 |
| 2kk | | <5 |
| 2ll | | <5 |
| 2mm | | <1 |
| 2nn | | <1 |
| 2oo | | <5 |
| 2pp | <20 | |
| 2qq | <5 | |
| 2rr | <5 | |
| 2ss | <5 | |
| 2tt | <1 | |
| 2uu | <20 | |
| 2vv | <20 | |
| 2ww | <20 | |
| 2xx | >40 | |
| 2yy | <20 | |
| 2zz | >40 | |
| 2aaa | <5 | |
| 2bbb | <1 | |
| 2ccc | 40 | |
| 2ddd | <5 | |
| 2eee | <5 | |
| 2fff | <5 | |
| 2ggg | <5 | |
| 2hhh | <40 | |
| 2iii | <5 | |
| 2jjj | <20 | |
| 2kkk | <5 | |
| 2lll | <5 | |
| 2mmm | <40 | |
| 2nnn | <40 | |
| 2ooo | >40 | |
| 3a | | <1 |
| 3b | <5 | |
| 3c | <5 | |
| 3d | <5 | |
| 3e | <5 | |
| 3f | <5 | |
| 3g | <10 | |
| 3g | <10 | |
| 3i | <40 | |
| 3j | <10 | |
| 3k | <10 | |
| 3l | <10 | |
| 3m | <5 | |
| 3n | <5 | |
| 3o | <20 | |
| 3p | <20 | |
| 3q | >40 | |
| 3r | <5 | |
| 3s | <5 | |
| 3t | <5 | |
| 3u | <10 | |
| 3v | <1 | |
| 3w | <5 | |
| 3x | <1 | |
| 3y | <1 | |
| 3z | <1 | |
| 3aa | <1 | |
| 3bb | >40 | |
| 4a | | >40 |
| 4b | >40 | |
| 4c | >40 | |
| 4d | | >40 |
| 4e | >40 | |
| 4f | <5 | |
| 4g | <5 | |
| 4h | >40 | |
| 4i | >40 | |
| 4j | >40 | |
| 4k | >40 | |
| 4l | <5 | |
| 4m | | <20 |
| 4n | <10 | <10 |
| 4o | <5 | |
| 4p | | <5 |
| 4q | | <1 |
| 4r | | <5 |
| 4s | | <1 |
| 4t | | <20 |
| 4u | | <10 |
| 4v | | 40 |
| 4w | | <1 |
| 4x | | >40 |
| 4y | <5 | |
| 4z | | >40 |
| 4aa | | 20 |
| 4bb | >40 | |
| 4cc | >40 | |
| 4dd | | >40 |
| 4ee | | >40 |
| 4ff | | >40 |

While the compositions and methods of this invention have been described in terms of illustrative embodiments, it will be apparent to those of skill in the art that variations may be applied to the compositions, methods and/or processes and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

What is claimed is:

1. A compound of Formula I, 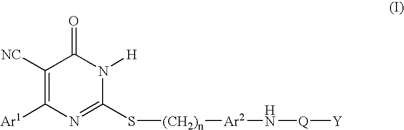
or a pharmaceutically acceptable salt, solvate, or ester thereof, wherein:
Ar¹ is a mono- or bicyclic aryl or a mono- or bicyclic heteroaryl, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
Ar² is a mono- or bicyclic aryl or a mono- or bicyclic heteroaryl all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
n is an integer from 1 to 6;
Q is (CH₂)_{q}O(CH₂)ₜ or a straight chain, branched or cyclic alkyl from 1 to 10 carbon atoms, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
q is an integer from 1 to 4;
t is 0 or an integer from 1 to 4; and
Y is a mono- or bicyclic aryl or a mono- or bicyclic heteroaryl which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol.

2. A compound of Formula II, 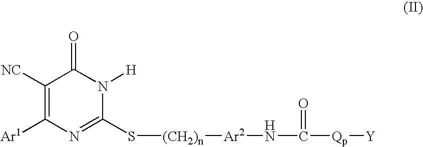
or a pharmaceutically acceptable salt, solvate, or ester thereof,
wherein:
Ar¹ is a mono- or bicyclic aryl or a mono- br bicyclic heteroaryl, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
Ar² is a mono- or bicyclic aryl or a mono- or bicyclic heteroaryl all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
n is an integer from 1 to 6;
Q is (CH₂)_{q}O(CH₂)ₜ or a straight chain, branched or cyclic alkyl from 1 to 10 carbon atoms, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
q is an integer from 1 to 4;
t is 0 or an integer from 1 to 4;
p is 0 or 1; and
Y is a mono- or bicyclic aryl or a mono- or bicyclic heteroaryl which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol.

3. A compound of Formula (III), 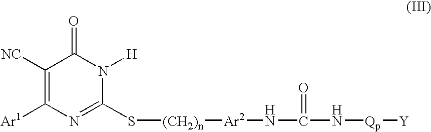
or a pharmaceutically acceptable salt, solvate, or ester thereof,
wherein:
Ar¹ is a mono- or bicyclic aryl or a mono- or bicyclic heteroaryl, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
Ar² is a mono- or bicyclic aryl or a mono- or bicyclic heteroaryl all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
n is an integer from 1 to 6;
Q is (CH₂)_{q}O(CH₂)ₜ or a straight chain, branched or cyclic alkyl from 1 to 10 carbon atoms, all of which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol;
q is an integer from 1 to 4;
t is 0 or an integer from 1 to 4;
p is 0 or 1; and
Y is a mono- or bicyclic aryl or a mono- or bicyclic heteroaryl which can be optionally substituted with one or more substituents independently selected from the group consisting of hydroxyl, thiol, halo, nitro, cyano, alkyl, alkenyl, alkynyl, aryl, heteroaryl, heterocycle, carbocycle, haloalkyl, hydroxyalkyl, aminoalkyl, aralkyl, cycloalkyl, polyoxyalkylene, polyol alkyl, alkylcarbonylalkyl, lower alkyl S(O)-lower alkyl, lower alkyl-S(O)₂-lower alkyl, aralkyl lower thioalkyl, heteroaralkyl lower thioalkyl, heterocyclealkyl lower thioalkyl, heteroaryl lower alkyl, heterocycle lower alkyl, heteroarylthio lower alkyl, arylthio lower alkyl, heterocyclethio lower alkyl, heteroarylamino lower alkyl, heterocycleamino lower alkyl, arylsulfinyl lower alkyl, and arylsulfonyl lower alkyl, alkoxy, haloalkoxy, alkylaminoalkoxy, aminoalkoxy, arylaminoalkoxy, heteroarylaminoalkoxy, heterocycleaminoalkoxy, acyloxy, aryloxy, arylalkoxy, heteroaryloxy; heteroarylalkoxy, heterocycleoxy, heterocyclealkoxy, heteroaryl lower alkoxy, heterocycle lower alkoxy, alkylthio, haloalkylthio, thioether, amino, alkylamino, dialkylamino, alkylsulfonylamino, acylamino, arylamino, heteroarylamino, heterocycleamino, oxyalkylamino, amido, imide, sulfonylimide, carboxamido, sulfonamido, amino acid, amino acid esters, amino acid amides, acyl, aminoacyl, carboxyl, carboxylic ester, carboxylic acid, carbamate, sulfonyl, alkylsulfonyl, arylsulfonyl, aminosulfonyl, haloalkylsulfonyl, thioester, hydroxamic acid, tetrazolyl, carbohydrate, or alditol.

4. A pharmaceutical composition for the treatment of a disorder selected from an inflammatory disorder, a cardiovascular disorder and abnormal cell proliferation in a subject, comprising an effective amount of a compound of one of claims 1, 2 or 3 in a pharmaceutically acceptable carrier.

5. The composition of claim 4 wherein the disorder is selected from arthritis, osteoarthritis, rheumatoid arthritis, asthma, dermatitis, cystic fibrosis, post transplantation late and chronic solid organ rejection, multiple sclerosis, systemic lupus erythematosis, inflammatory bowel diseases, autoimmune diabetes, ophthalmologic disorders associated with inflammation, diabetic retinopathy, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina and small artery disease, atherosclerosis, post-angioplasty restenosis, coronary artery disease, angina.

6. The composition of claim 4 wherein the disorder is selected from an inflammatory skin disease, psoriasis, dermatitis, eczematous dermatitis, Kaposi's sarcoma, multiple sclerosis, or a proliferative disorder of smooth muscle cells.

7. The composition of claim 4 wherein the disorder is mediated by the activation of one or more protein kinases and in which surgery is difficult or not an option.

8. The composition of claim 4 wherein the composition is suitable for oral, parenteral, or intravenous delivery.

9. A method of treating a disorder selected from an inflammatory disorder, a cardiovascular disorder and abnormal cell proliferation in a host comprising administering a compound of one of claims 1, 2 or 3 to the host, optionally in a pharmaceutically acceptable carrier.

10. The method of claim 9 wherein the disorder is selected from arthritis, osteoarthritis, rheumatoid arthritis, asthma, dermatitis, cystic fibrosis, post transplantation late and chronic solid organ rejection, multiple sclerosis, systemic lupus erythematosis, inflammatory bowel diseases, autoimmune diabetes, ophthalmologic disorders associated with inflammation, diabetic retinopathy, rhinitis, ischemia-reperfusion injury, post-angioplasty restenosis, chronic obstructive pulmonary disease (COPD), glomerulonephritis, Graves disease, gastrointestinal allergies, conjunctivitis, atherosclerosis, coronary artery disease, angina and small artery disease, atherosclerosis, post-angioplasty restenosis, coronary artery disease, angina.

11. The method of claim 9 wherein the disorder is selected from an inflammatory skin disease, psoriasis, dermatitis, eczematous dermatitis, Kaposi's sarcoma, multiple sclerosis, or a proliferative disorder of smooth muscle cells.

12. The method of claim 9 wherein the disorder is mediated by the activation of one or more protein kinases and in which surgery is difficult or not an option.

13. The method of claim 9 wherein the compound is administered via oral, parenteral, or intravenous delivery.
